(19) United States
(12) Patent Application Publication
(10) Pub. No.: US 2022/0056460 A1
(43) Pub. Date: Feb. 24, 2022

(54) CRISPR GUIDE-RNA EXPRESSION STRATEGIES FOR MULTIPLEX GENOME ENGINEERING

(71) Applicant: DSM IP ASSETS B.V., Heerlen (NL)

(72) Inventors: Rene VERWAAL, Echt (NL); Nathalie WIESSENHAAN, Echt (NL); Johannes Andries ROUBOS, Echt (NL)

(21) Appl. No.: 17/299,413

(22) PCT Filed: Nov. 29, 2019
(86) PCT No.: PCT/EP2019/083018

§ 371 Date: Jun. 3, 2021

(30) Foreign Application Priority Data
Dec. 5, 2018 (EP) 18210371.3

(51) Int. Cl. C12N 15/81; C12N 15/11; C12N 1/16; C12N 15/10

(52) U.S. Cl. CPC C12N 15/81; C12N 15/111; C12N 1/16; C12N 15/1024; C12R 2001/865; C12N 2310/20; C12N 2310/3519; C12N 2310/51; C12N 2330/51; C12N 2310/128

(57) ABSTRACT

The invention relates to the field of molecular biology and cell biology. More specifically, the invention relates to CRISPR guide-RNA expression strategies for multiplex genome engineering.

### FIELD

The invention relates to the field of molecular biology and cell biology. More specifically, the invention relates to CRISPR guide-RNA expression strategies for multiplex genome engineering.

### BACKGROUND

Different strategies for multiplex guide-RNA (gRNA) expression for genome editing and transcriptional regulation are known in the art, such as multiplex guide-RNA (gRNA) expression in S. cerevisiae (Lian et al., 2018). Multiplex expression of an array of single guide-RNAs (sgRNA) was achieved using ribozymes sequences (Gao and Zhao, 2014) that flank the sgRNAs (RGR, ribozyme-sgRNA-ribozyme, FIG. 1A, FIG. 2A). The ribozyme sequences will be self-processed and at least four sgRNAs have been successfully expressed using this Pol II-RGR system (Deaner et al., 2017). Another way to express multiple guides from one transcript is by exploiting the RNA processing capacity of the bacterial endoribonuclease Csy4 from Pseudomonas aeruginosa (Nissim et al., 2014). Multiple gRNAs sequences are then flanked by recognition motifs for Csy4 (FIG. 1B, FIG. 2B). In combination with a RNA pol II promoter, at least three gRNAs can be fully transcribed and processed (Lian et al., 2017). In combination with a RNA pol III promoter, multiplexed CRISPR/Cas9 genome editing and gene interference applications in S. cerevisiae were demonstrated to express at least three gRNAs (Ferreira et al., 2018). In both cases, co-expression of Csy4 is required to process the Pol II/Pol III-Csy4-gRNA arrays. Recently it was demonstrated that multiple sgRNA expression is enabled by the tRNA-gRNA-tRNA (TST) expression system, where tRNAs are used as splicing elements flanking gRNAs expressed from a Pol II promoter, demonstrated for the expression of up to two gRNAs (Deaner et al., 2018, FIG. 10, FIG. 2C). While Cas9 is currently the best characterized and most widely used nuclease as a versatile tool for genome editing and gene regulation applications, Cas12a (previously named Cpf1 (Makarova et al., 2017)) has recently emerged as an alternative for Cas9 (Knott and Doudna, 2018; Schwarts and Jinek, 2018). Cpf1 is a class 2/type V RNA-guided endonuclease discovered in several bacterial genomes and one archaeal genome (Makarova et al., 2015). CRISPR/Cpf1 genome editing has been evaluated in human cells (Zetsche et al., 2015; Kim D et al., 2016), mice (Hur et al., 2016; Kim Y et al., 2016), Drosophila (Port and Bullock, 2016), rice (Xu et al., 2017) and plant cells (Kim H et al., 2017; Mahfouz, 2017). Interestingly, several features of the CRISPR/Cpf1 system are different compared with CRISPR/Cas9 (Zetsche et al., 2015): (1) Cpf1 recognizes T-rich PAM sequences, i.e. 5′-TTTN-3′ (AsCpf1, LbCpf1) and 5′-TTN-3′ (FnCpf1), whereas this is NGG for SpCas9. The PAM preference for AsCpf1 and LbCpf1 was proposed to be TTTV (Kim H K et al., 2017). (2) Cpf1 is characterized by a PAM sequence located at the 5′ end of the target DNA sequence, where it is at the 3′ end for Cas9. (3) Cpf1 cleaves DNA distal to its PAM after the +18/+23 position of the protospacer creating a staggered DNA overhang, whereas Cas9 cleaves close to its PAM after the −3 position of the protospacer at both strands and creates blunt ends. (4) Cpf1 is guided by a single crRNA and does not require a tracrRNA, resulting in a shorter gRNA sequence than the gRNA used by Cas9. The single crRNA is composed of a direct repeat sequence followed by a spacer (or guide) sequence. (5) Cpf1 displays an additional ribonuclease activity that functions in crRNA processing (Fonfara et al., 2016). This might simplify multiplex genome editing, as demonstrated by Zetsche et al., (2017) who used a single crRNA array to simultaneously edit up to four genes in mammalian cells. A single crRNA array was also used for multiplex genome editing of rice (Wang et al., 2017). It was demonstrated that multiplex genome editing using a single LbCpf1 crRNA array (Verwaal et al., 2018, FIG. 1D, FIG. 2D), or using a single FnCpf1 crRNA array (Swiat et al., 2017), was also functional in the yeast Saccharomyces cerevisiae.

Research in mammalian cells has shown that shortening the direct repeat in the single AsCpf1 crRNA array by one nucleotide from a 20 bp direct repeat (Zetsche et al., 2015) to 19 bp and using a 23 bp guide gave the best results (Zetsche et al., 2017). A single LbCpf1 crRNA in Saccharomyces cerevisiae, composed of LbCpf1 direct repeat sequences shorted by 1 nucleotide, from 21 bp (Zetsche et al., 2015) to 20 bp and a 23 bp guide sequence, was successfully used for CRISPR/Cpf1 multiplex engineering (Verwaal et al., 2018). Swiat et al., 2017 demonstrated that a single FnCpf1 crRNA array composed of 19 nt direct repeat sequences, shortening the direct repeat by one nucleotide, and using a 25 bp guide sequence, enabled CRISPR/Cpf1 multiplex genome editing in S. cerevisiae. Thus, the direct repeat and guide nucleotide lengths as schematically depicted in FIG. 3 may differ between Cpf1 variants (e.g. AsCpf1, LbCpf1, FnCpf1).

For efficient multiplex genome engineering, there is a need to improve construction of multiple guide nucleotide expression DNA constructs. As a default, CRISPR arrays would be chemically synthesized as linear dsDNA by commercial vendors. Unfortunately, the reoccurring repeat sequences inherent to these arrays currently pose major technical complications when assembling individually synthesized oligonucleotides, resulting in vendors regularly rejecting customer requests even for a minimal single-spacer array. Gene synthesis has offered a more reliable means of obtaining custom CRISPR arrays. However, synthesis often comes at large cost (˜5× the price of a linear dsDNA) and timeframes (˜1 month), and the synthesis can often fail.

As an alternative, a few groups have developed different in vitro assembly methods based on annealing shorter oligonucleotides into repeat-spacer subunits that can be assembled sequentially or simultaneously into arrays (Cress et al., 2015; Gomaa et al., 2014; Tak et al., 2017; Vercoe et al., 2013; Zetsche et al., 2017). For instance, one study sequentially inserted individual repeat-spacer subunits into a non-target spacer to generate Cas9 arrays with up to a three spacers (Cress et al., 2015) while another assembled Cas12a arrays with up to three spacers in one step by creating 5′ overhangs that fall within different parts of the conserved repeat (Tak et al., 2017). While these approaches were used to successfully generate CRISPR arrays harboring 2-4 spacers, they cannot scale to larger arrays and often exhibited low cloning efficiencies even for these small arrays. Another in vitro assembly approach was described by Liao et al., 2018, who presented an assembly scheme for the efficient, one-step generation of large CRISPR arrays. The method, which was named CRATES (CRISPR Assembly through Trimmed Ends of Spacers), relies on ligating ˜60-nt repeat-spacer units at defined junctions within the trimmed and therefore expendable portion of each spacer. The junctions allowed for the efficient assembly of arrays with up to seven spacers (Liao et al., 2018). Therefore, the ability to easily, cheaply, and quickly generate CRISPR arrays remains an impediment to the widespread use of CRISPR multiplexing and the fundamental study of gRNA array processing and function.

The invention provides an improvement as compared to chemical DNA synthesis and in vitro assembly approaches.

### SUMMARY

Provided is the use of a plurality of single-stranded oligonucleotide members and a linear double-stranded polynucleotide member in the assembly within a cell of a double-stranded polynucleotide construct of pre-determined sequence, wherein the members of the plurality of single-stranded oligonucleotides are capable of assembly within a cell into a double-stranded polynucleotide encoding an array of at least two functional guide-RNA molecules, wherein each guide-RNA molecule comprises at least a guide sequence and an RNA processing sequence.

Further provided is a method for assembly within a cell, of a double-stranded polynucleotide construct of pre-determined sequence comprising contacting a cell with a plurality of single-stranded oligonucleotide members and a linear double-stranded polynucleotide member such that these are introduced into the cell, wherein the members of the plurality of single-stranded oligonucleotides are capable of assembly within a cell into a double-stranded polynucleotide encoding an array of at least two functional guide-RNA molecules, wherein each guide-RNA molecule comprises at least a guide sequence and an RNA processing sequence.

Further provided is a method for expression, within a cell of at least two functional guide-RNA molecules, comprising contacting a cell having Cas12a-like enzyme activity with a plurality of single-stranded oligonucleotide members and a linear double-stranded polynucleotide member such that these are introduced into the cell, wherein the members of the plurality of single-stranded oligonucleotides are capable of assembly within a cell into a double-stranded polynucleotide encoding an array of at least two functional guide-RNA molecules, wherein each guide-RNA molecule comprises at least a guide sequence and an RNA processing sequence.

Further provided is a method for gene editing, comprising contacting a cell with a plurality of single-stranded oligonucleotide members and a linear double-stranded polynucleotide member such that these are introduced into the cell, wherein the members of the plurality of single-stranded oligonucleotides are capable of assembly within a cell into a double-stranded polynucleotide encoding an array of at least two functional guide-RNA molecules, wherein each guide-RNA molecule comprises at least a guide sequence and an RNA processing sequence.

Further provided is a double-stranded polynucleotide encoding an array of at least two guide-RNA molecules obtainable or obtained by a method for assembly, expression or gene editing as disclosed herein, wherein each guide-RNA molecule comprises at least a guide sequence and an RNA processing sequence.

Further provided is a cell obtainable by or obtained by a method for assembly, expression or gene editing as disclosed herein.

Further provided is a method for the production of a double-stranded polynucleotide encoding an array of at least two guide-RNA molecules obtainable or obtained by a method for assembly, expression or gene editing as disclosed herein, wherein each guide-RNA molecule comprises at least a guide sequence and an RNA processing sequence, said method comprising, performing a method for assembly, expression or gene editing as disclosed herein and subsequently isolating the double-stranded polynucleotide from the cell.

Further provided is a method for the production of a compound of interest comprising, culturing a cell as disclosed herein, said cell comprising a polynucleotide encoding a compound of interest, under conditions conducive to the production of the compound of interest, and optionally isolating and/or purifying the compound of interest.

### BRIEF DESCRIPTION OF THE DRAWINGS

FIG. 1 depicts CRISPR guide-RNA expression strategies for multiplex genome engineering. A) A single RNA polymerase type II promoter can be used to drive the expression of an array of multiple sgRNAs, which are flanked by ribozymes (HDV: hepatitis delta virus ribozyme; HH: hammerhead ribozyme). This approach can be used in combination with a Cas9-like enzyme. B) A single type II or type III promoter can be used to drive the expression of an array of multiple sgRNAs, which are flanked by Csy4 cutting sites. This approach can be used in combination with a Cas9-like enzyme. C) A single type II promoter can be used to drive the expression of an array of multiple gRNAs, which are flanked by transfer-RNA (tRNA) sequences. This approach can be used in combination with a Cas9-like enzyme. D) A single type III promoter can be used to drive the expression of an array of multiple crRNAs (single crRNA array). This approach can be used in combination with a Cas12a-like enzyme.

FIG. 2 depicts CRISPR guide-RNA expression strategies for multiplex genome engineering. Homology to a recipient vector, for example pRN1120, to allow in vivo recombination of the gRNA arrays of A), B) or C) or the single crRNA array of D) into the recipient, linearized vector in S. cerevisiae, is indicated as striped boxes. A) A single RNA polymerase type II promoter can be used to drive the expression of an array of multiple sgRNAs, which are flanked by ribozymes (HDV: hepatitis delta virus ribozyme; HH: hammerhead ribozyme). This approach can be used in combination with a Cas9-like enzyme. B) A single type II or type III promoter can be used to drive the expression of an array of multiple sgRNAs, which are flanked by Csy4 cutting sites. This approach can be used in combination with a Cas9-like enzyme. C) A single type II promoter can be used to drive the expression of an array of multiple gRNAs, which are flanked by transfer-RNA (tRNA) sequences. This approach can be used in combination with a Cas9-like enzyme. D) A single type III promoter can be used to drive the expression of an array of multiple crRNAs (single crRNA array). This approach can be used in combination with a Cas12a-like enzyme.

FIG. 3 depicts a schematic representation of the single crRNA array expression cassette for Cpf1/Cas12a and processing to individual crRNAs by Cpf1, which could be applied for different Cpf1 orthologues like Acidaminococcus spp. BV3L6 Cpf1 (AsCpf1), Lachnospiraceae bacterium ND2006 Cpf1 (LbCpf1) or Francisella novicida U112 Cpf1 (FnCpf1). For example, the single LbCpf1 crRNA array used in Examples 1 and 2 (LbCpf1_crRNA_array) is composed of three units of crRNAs in their mature form, a 20 bp direct repeat specific for LbCpf1 (DR, grey boxes) with a 23 bp guide sequence (white diamond: target to INT1, black diamond: target to INT2, horizontal striped diamond: target to INT3). Expression of the crRNA array is enabled by the SNR52 promoter (SNR52p) and SUP4 terminator (T).

FIG. 4 depicts a schematic representation of the single crRNA array expression cassette for Cpf1/Cas12a and processing to individual crRNAs by Cpf1, which could be applied for different Cpf1 orthologues like Acidaminococcus spp. BV3L6 Cpf1 (AsCpf1), Lachnospiraceae bacterium ND2006 Cpf1 (LbCpf1) or Francisella novicida U112 Cpf1 (FnCpf1). For example, the single LbCpf1 crRNA array used in Examples 1 and 2 (LbCpf1_crRNA_array) is composed of three units of crRNAs in their mature form, a 20 bp direct repeat specific for LbCpf1 (DR, grey boxes) with a 23 bp guide sequence (white diamond: target to INT1, black diamond: target to INT2, horizontal striped diamond: target to INT3). Expression of the crRNA array is enabled by the SNR52 promoter (SNR52p) and SUP4 terminator (T). Homology to a recipient vector, for example pRN1120 (striped boxes), to allow in vivo recombination of the single crRNA array into the vector in S. cerevisiae as shown in FIG. 8, is also depicted.

FIG. 5 depicts the vector map of multicopy (2 micron) vector pRN1120. A NatMX marker is present on the vector.

FIG. 6 depicts the nucleotide sequences of the different DNA elements part of the LbCp1_crRNA_array expression cassette to enable CRISPR/Cpf1 mediated multiplex genome editing in S. cerevisiae.

FIG. 7 depicts the vector map of single copy (CEN/ARS) vector pCSN067 expressing LbCpf1. LbCpf1 was codon-pair optimized for expression in S. cerevisiae according the method described in WO2008/000632. A KanMX marker is present on the vector.

FIG. 8 depicts in vivo recombination in S. cerevisiae of linearized pRN1120 and the LbCpf1_crRNA_array that contains homology with pRN1120 (striped boxes). As vector pRN1120 contains a NatMX marker, the resulting circular vector allows for selection on nourseothricin after transformation.

FIG. 9 depicts a schematic representation of CRISPR/Cpf1 multiplex genome editing using a single LbCpf1_crRNA_array. Cpf1 is directed to the intended INT1, INT2 and INT3 genomic target sites by crRNA_1, crRNA_2 and crRNA_3, respectively, to create double-stranded breaks. In the transformation mixture, donor DNA consisting of flank sequences and carotenoid gene expression cassettes were included. All donor DNAs integrate into genomic DNA of the INT1 (crtE), INT2 (crtYB) and INT3 (crtl) loci facilitated by in vivo recombination of 50 bp homologous connector sequences, indicated as 5, A, B, C, D and E, respectively, and homology of the donor DNA flank sequences with genomic DNA.

FIG. 10 depicts the PCR results to determine correct integration of crtE in the INT1 locus, crtYB in the INT2 locus and crtl in the INT3 locus, using the single LbCpf1_crRNA_array. The PCR was performed on genomic DNA template isolated from transformation 1 and 2 and from control 1 and 2. When integration is correct, the following bands appear on the agarose gel. Band A: correct integration of crtE at the INT1 locus 5′ end (2295 bp). Band B: Correct integration of crtE at the INT1 locus 3′ end (1812 bp). Band E: Correct integration of crtYB at the INT2 locus 5′ end (3406 bp). Band F: Correct integration of crtYB at the INT2 locus 3′ end (1814 bp). Band C: Correct integration of crtl at the INT3 locus 5′ end (2544 bp). Band D: Correct integration of crtl at the INT3 locus 3′ end (1817 bp). A 1 Kb plus ladder was also loaded on the gel. Further details can be found in Table 3.

FIG. 11 depicts a vector map of multi copy (2 micron) vector pGRN002, containing the SNR52 polymerase III promoter (SNR52p), a guide-RNA structural component specific for SpCas9 and SUP4 terminator (SUP4t) sequences. After restriction with SapI and XhoI, a Cpf1 crRNA array can be assembled into the linear vector using oligonucleotides by in vivo assembly in S. cerevisiae to generate a crRNA expression cassette as explained in Example 2. A NatMX (nourseothricin) resistance marker is present on the vector.

FIG. 12 depicts the in vivo assembly approach using oligonucleotides that assemble into the SapI/XhoI a linearized vector, for example pGRN002, to constitute a crRNA array expression cassette in vivo in S. cerevisiae. After assembly, the LbCpf1_crRNA_array is composed of three units of crRNAs in their mature form, a 20 bp direct repeat specific for LbCpf1 (DR_Lb, grey boxes) with a 23 bp guide sequence (white diamond: INT1 guide, black diamond: INT2 guide, horizontal striped diamond: INT3 guide). Expression of the crRNA array is enabled by the SNR52 promoter (SNR52p) and SUP4 terminator (T). A) Oligo variant 1, example where 8 oligonucleotides were used to constitute the crRNA array expression cassette with three crRNAs. Details on the oligonucleotide sequences are depicted in FIG. 13A). B) Oligo variant 2, example where 7 oligonucleotides were used to constitute the crRNA array expression cassette with three crRNAs. Details on the oligonucleotide sequences are depicted in FIG. 13B).

FIG. 13 depicts the sequence details of the oligonucleotides that assemble into SapI/XhoI linearized vector pGRN002 to constitute a crRNA array expression cassette in vivo in S. cerevisiae. Direct repeat sequences are indicated within the closed squares. The sequence for SUP4t is indicated in the dashed square. Homology to the SNR52p, vector pGRN002 and three spacer/guide sequences (INT1 guide, INT2 guide, INT3 guide) is indicated. A) Oligo variant 1, example where 8 oligonucleotides were used to constitute the crRNA array expression cassette with three crRNAs. B) Oligo variant 2, example where 7 oligonucleotides were used to constitute the crRNA array expression cassette with three crRNAs.

### DESCRIPTION OF THE SEQUENCES

SEQ ID NO: 1 sets out the nucleotide sequence of vector pRN1120.

SEQ ID NO: 2 sets out the nucleotide sequence of the LbCpf1_crRNA_array nucleotide sequence including homology with plasmid pRN1120.

SEQ ID NO: 3 sets out the nucleotide sequence of the FW primer to amplify a LbCpf1 crRNA array expression cassette for in vivo assembly into linearized pRN1120.

SEQ ID NO: 4 sets out the nucleotide sequence of the REV primer to amplify a LbCpf1 crRNA array expression cassette for in vivo assembly into linearized pRN1120.

SEQ ID NO: 5 sets out the nucleotide sequence of the synthetic and donor DNA crtE expression cassette (con5-KITDH2p-crtE-ScTDH3t-conA). This nucleotide sequence was ordered as synthetic DNA, it served as template for PCR and this nucleotide sequence is also the sequence that was used in the transformation of this donor DNA expression cassette.

SEQ ID NO: 6 sets out the nucleotide sequence of the synthetic crtYB expression cassette (conA-KIYDR2p-crtYB-ScPDC1t-conB).

SEQ ID NO: 7 sets out the nucleotide sequence of the synthetic crtl expression cassette (conB-ScPRE3p-crtl-ScTAL1t-conC).

SEQ ID NO: 8 sets out the nucleotide sequence of the donor DNA crtYB expression cassette (conB-KIYDR2p-crtYB-ScPDC1t-conC).

SEQ ID NO: 9 sets out the nucleotide sequence of the donor DNA crtl expression cassette (conD-ScPRE3p-crtl-ScTAL1t-conE).

SEQ ID NO: 10 sets out the nucleotide sequence of the FW primer to obtain the con5-crtE-conA donor DNA expression cassette, integration into INT1.

SEQ ID NO: 11 sets out the nucleotide sequence of the REV primer to obtain the con5-crtE-conA donor DNA expression cassette, integration into INT1.

SEQ ID NO: 12 sets out the nucleotide sequence of the FW primer to obtain the conB-crtYB-conC donor DNA expression cassette, integration into INT2.

SEQ ID NO: 13 sets out the nucleotide sequence of the REV primer to obtain the conB-crtYB-conC donor DNA expression cassette, integration into INT2.

SEQ ID NO: 14 sets out the nucleotide sequence of the FW primer to obtain the conD-crtl-conE donor DNA expression cassette, integration into INT3.

SEQ ID NO: 15 sets out the nucleotide sequence of the REV primer to obtain the conD-crtl-conE donor DNA expression cassette, integration into INT3.

SEQ ID NO: 16 sets out the nucleotide sequence of the INT1 5′-con5 donor DNA flank sequence.

SEQ ID NO: 17 sets out the nucleotide sequence of the conA-INT1 3′ donor DNA flank sequence.

SEQ ID NO: 18 sets out the nucleotide sequence of the INT2 5′-conB donor DNA flank sequence.

SEQ ID NO: 19 sets out the nucleotide sequence of the conC-INT2 3′ donor DNA flank sequence.

SEQ ID NO: 20 sets out the nucleotide sequence of the INT3 5′-conD donor DNA flank sequence.

SEQ ID NO: 21 sets out the nucleotide sequence of the conE-INT3 3′ donor DNA flank sequence.

SEQ ID NO: 22 sets out the nucleotide sequence of the FW primer to obtain the INT1 5′-con5 donor flank sequence.

SEQ ID NO: 23 sets out the nucleotide sequence of the REV primer to obtain the INT1 5′-con5 donor flank sequence.

SEQ ID NO: 24 sets out the nucleotide sequence of the FW primer to obtain the conA-INT1 3′ donor flank sequence.

SEQ ID NO: 25 sets out the nucleotide sequence of the REV primer to obtain the conA-INT1 3′ donor flank sequence.

SEQ ID NO: 26 sets out the nucleotide sequence of the FW primer to obtain the INT2 5′-conB donor flank sequence.

SEQ ID NO: 27 sets out the nucleotide sequence of the REV primer to obtain the INT2 5′-conB donor flank sequence.

SEQ ID NO: 28 sets out the nucleotide sequence of the FW primer to obtain the conC-INT2 3′ donor flank sequence.

SEQ ID NO: 29 sets out the nucleotide sequence of the REV primer to obtain the conC-INT2 3′ donor flank sequence.

SEQ ID NO: 30 sets out the nucleotide sequence of the FW primer to obtain the INT3 5′-conD donor flank sequence.

SEQ ID NO: 31 sets out the nucleotide sequence of the REV primer to obtain the INT3 5′-conD donor flank sequence.

SEQ ID NO: 32 sets out the nucleotide sequence of the FW primer to obtain the conE-INT3 3′ donor flank sequence.

SEQ ID NO: 33 sets out the nucleotide sequence of the REV primer to obtain the conE-INT3 3′ donor flank sequence.

SEQ ID NO: 34 sets out the nucleotide sequence of the FW primer to check correct integration of crtE at the INT1 locus 5′ end (band A).

SEQ ID NO: 35 sets out the nucleotide sequence of the REV primer to check correct integration of crtE at the INT1 locus 5′ end (band A).

SEQ ID NO: 36 sets out the nucleotide sequence of the FW primer to check correct integration of crtE at the INT1 locus 3′ end (band B).

SEQ ID NO: 37 sets out the nucleotide sequence of the REV primer to check correct integration of crtE at the INT1 locus 3′ end (band B).

SEQ ID NO: 38 sets out the nucleotide sequence of the FW primer to check correct integration of crtl at the INT3 locus 5′ end (band C).

SEQ ID NO: 39 sets out the nucleotide sequence of the REV primer to check correct integration of crtl at the INT3 locus 5′ end (band C).

SEQ ID NO: 40 sets out the nucleotide sequence of the FW primer to check correct integration of crtl at the INT3 locus 3′ end (band D).

SEQ ID NO: 41 sets out the nucleotide sequence of the REV primer to check correct integration of crtl at the INT3 locus 3′ end (band D).

SEQ ID NO: 42 sets out the nucleotide sequence of the FW primer to check correct integration of crtYB at the INT2 locus 5′ end (band E).

SEQ ID NO: 43 sets out the nucleotide sequence of the REV primer to check correct integration of crtYB at the INT2 locus 5′ end (band E).

SEQ ID NO: 44 sets out the nucleotide sequence of the FW primer to check correct integration of crtYB at the INT2 locus 3′ end (band F).

SEQ ID NO: 45 sets out the nucleotide sequence of the REV primer to check correct integration of crtYB at the INT2 locus 3′ end (band F).

SEQ ID NO: 46 sets out the nucleotide sequence of the FW primer to remove SapI restriction site in pRN1120.

SEQ ID NO: 47 sets out the nucleotide sequence of the REV primer to remove SapI restriction site in pRN1120.

SEQ ID NO: 48 sets out the nucleotide sequence of the gBlock to enable direct SapI cloning of a genomic target for SpCas9. The sequence is part of vector pGRN002.

SEQ ID NO: 49 sets out the nucleotide sequence of the nucleotide sequence of vector pGRN002.

SEQ ID NO: 50 sets out the nucleotide sequence of the FW oligonucleotide named FW oligo 1 for oligo assembly variant 1 and 2.

SEQ ID NO: 51 sets out the nucleotide sequence of the FW oligonucleotide named FW oligo 2 for oligo assembly variant 1 and 2.

SEQ ID NO: 52 sets out the nucleotide sequence of the FW oligonucleotide named FW oligo 3 for oligo assembly variant 1 and 2.

SEQ ID NO: 53 sets out the nucleotide sequence of the FW oligonucleotide named FW oligo 4 for oligo assembly variant 1 and 2.

SEQ ID NO: 54 sets out the nucleotide sequence of the REV oligonucleotide named REV oligo 1 for oligo assembly variant 1.

SEQ ID NO: 55 sets out the nucleotide sequence of the REV oligonucleotide named REV oligo 2 for oligo assembly variant 1.

SEQ ID NO: 56 sets out the nucleotide sequence of the REV oligonucleotide named REV oligo 3 for oligo assembly variant 1 and 2.

SEQ ID NO: 57 sets out the nucleotide sequence of the REV oligonucleotide named REV oligo 4 for oligo assembly variant 1 and 2.

SEQ ID NO: 58 sets out the nucleotide sequence of the REV oligonucleotide named REV oligo 5 for oligo assembly variant 2.

SEQ ID NO: 59 sets out the nucleotide sequence of the 5′ homology to vector pRN1120 part of the LbCpf1_crRNA_array shown in SEQ ID NO: 2.

SEQ ID NO: 60 sets out the nucleotide sequence of the SNR52p RNA pol III promoter part of the LbCpf1_crRNA_array shown in SEQ ID NO: 2.

SEQ ID NO: 61 sets out the nucleotide sequence of the direct repeat (specific for LbCpf1) part of the LbCpf1_crRNA_array shown in SEQ ID NO: 2.

SEQ ID NO: 62 sets out the nucleotide sequence of the genomic target/spacer of the INT1 integration site part of the LbCpf1_crRNA_array shown in SEQ ID NO: 2.

SEQ ID NO: 63 sets out the nucleotide sequence of the genomic target/spacer of the INT2 integration site part of the LbCpf1_crRNA_array shown in SEQ ID NO: 2.

SEQ ID NO: 64 sets out the nucleotide sequence of the genomic target/spacer of the INT3 integration site part of the LbCpf1_crRNA_array shown in SEQ ID NO: 2.

SEQ ID NO: 65 sets out the nucleotide sequence of the SUP4 3′ terminator part of the LbCpf1_crRNA_array shown in SEQ ID NO: 2.

SEQ ID NO: 66 sets out the nucleotide sequence of the 3′ homology to vector pRN1120 part of the LbCpf1_crRNA_array shown in SEQ ID NO: 2.

SEQ ID NO: 67 sets out the Csy4 recognition site for the Csy4 endoribonuclease from Pseudomonas aeruginosa.

SEQ ID NO: 68 sets out the coding sequence of tRNAgly (tGGC)

SEQ ID NO: 69 sets out the 5′ leader sequence for the coding sequence of tRNAgly

SEQ ID NO: 70 sets out the coding sequence of tRNAglu (tTTC)

SEQ ID NO: 71 sets out the 5′ leader sequence for the coding sequence of tRNAglu

SEQ ID NO: 72 sets out the coding sequence of tRNAtyr (tAGC)

SEQ ID NO: 73 sets out the 5′ leader sequence for the coding sequence of tRNAtyr

SEQ ID NO: 74 sets out the coding sequence of tRNAarg (tCTT)

SEQ ID NO: 75 sets out the 5′ leader sequence for the coding sequence of tRNAarg

SEQ ID NO: 76 sets out the coding sequence of tRNAasn (tGTT)

SEQ ID NO: 77 sets out the 5′ leader sequence for the coding sequence of tRNAasn

SEQ ID NO: 78 sets out the coding sequence of tRNAile (tAAT)

SEQ ID NO: 79 sets out the 5′ leader sequence for the coding sequence of tRNAile

### DETAILED DESCRIPTION

The inventors set out to provide a technique for expedient production of a single guide-RNA expression cassette comprising an array of guide-RNAs. Benefits of the in vivo (within a cell) assembly technique are:

1) No PCR is required to obtain the single crRNA array expression cassette. After dissolving, the oligonucleotides ordered can be directly used in the transformation experiment.
2) Changing one of the genomic targets by another one requires changing just three oligonucleotides in the in vivo assembly.
3) Easy building of single crRNA arrays in a combinatorial or designed approach.
4) The number of spacer/genomic target sequences and direct repeats can be easily expanded to allow more than three multiplex genome editing events by expanding the number of oligonucleotides in the approach as described in Example 2 herein.
5) The technique can be used to constitute single crRNA arrays for shuttle use in other microorganisms. Upon assembly of the single crRNA array in e.g. S. cerevisiae, a PCR could be performed to obtain a PCR fragment of the single crRNA array expression cassette, that can be cloned into the recipient guide expression vector, or recombined in vivo into a recipient vector of the host choice such as e.g. Aspergillus niger or Yarrowia lipolytica.

In a first aspect there is provided, the use of a plurality of single-stranded oligonucleotide members and a linear double-stranded polynucleotide member in the assembly within a cell of a double-stranded polynucleotide construct of pre-determined sequence, wherein the members of the plurality of single-stranded oligonucleotides are capable of assembly within a cell into a double-stranded polynucleotide encoding an array of at least two functional guide-RNA molecules, wherein each guide-RNA molecule comprises at least a guide sequence and an RNA processing sequence.

A polynucleotide, an oligonucleotide and a cell are defined in the section “Definitions” herein. The terms “assembly” and interchangeably “assembly within a cell” mean that two or more oligonucleotides or polynucleotides aggregate together within a cell by base paring to form a single construct which construct is processed by the cell into a double-stranded polynucleotide. A plurality of single-stranded oligonucleotide members means at least two single-stranded oligonucleotide members. A double-stranded polynucleotide encoding an array of at least two functional guide-RNA molecules means that the double-stranded polynucleotide is an expression construct that comprises all necessary coding and non-coding sequences (non-coding sequences such as control sequences) to produce, upon expression, an array of at least two functional guide-RNA molecules, wherein each guide-RNA molecule comprises at least a guide sequence and an RNA processing sequence. Thus the double-stranded polynucleotide will comprise sequences coding for the at least two functional guide-RNA molecules, wherein each guide-RNA molecule comprises at least a guide sequence and an RNA processing sequence. After expression, the array of at least two functional guide-RNA molecules is processed into individual functional guide-RNA molecules, facilitated by the RNA processing sequences. An array of at least two functional guide-RNA molecules means one contiguous RNA molecule comprising the at least two functional guide-RNA molecules. Guide RNA molecules have been described extensively and are known to the person skilled in the art (e.g. Mali et al., 2013; Cong et al., 2013; Zetsche et al., 2015; Gao and Zhao, 2014). Any functional guide RNA comprises at least a guide-sequence. A guide-sequence herein is a part of the guide-RNA that is able to hybridize with a target-sequence in a target-polynucleotide such as a target-genome and is able to direct sequence-specific binding of a genome editing system to the target-polynucleotide. The guide-RNA is a polynucleotide according to the general definition of a polynucleotide set out herein. A guide-sequence is herein also referred as a target-sequence and is essentially the complement of a target-polynucleotide such that the guide-polynucleotide is able to hybridize with the target-polynucleotide, preferably under physiological conditions in a host cell. The degree of complementarity, when optimally aligned using a suitable alignment algorithm, is preferably higher than 50%, 60%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity. The sequence identity may be 100%. Pre-determined sequence means that the sequence of the resulting double-stranded polynucleotide construct has been designed before application of the methods and use disclosed herein. An RNA processing sequence is herein defined as a sequence within the array of guide-RNA molecules that facilitates processing of the array of at least two functional guide-RNA molecules into separate functional guide-RNA molecules.

The RNA processing sequence may be any sequence with RNA processing capacities as defined herein above, such as a Cas12a Direct Repeat (DR) sequence, a Csy4 recognition sequence, a self-processing ribozyme or a tRNA sequence.

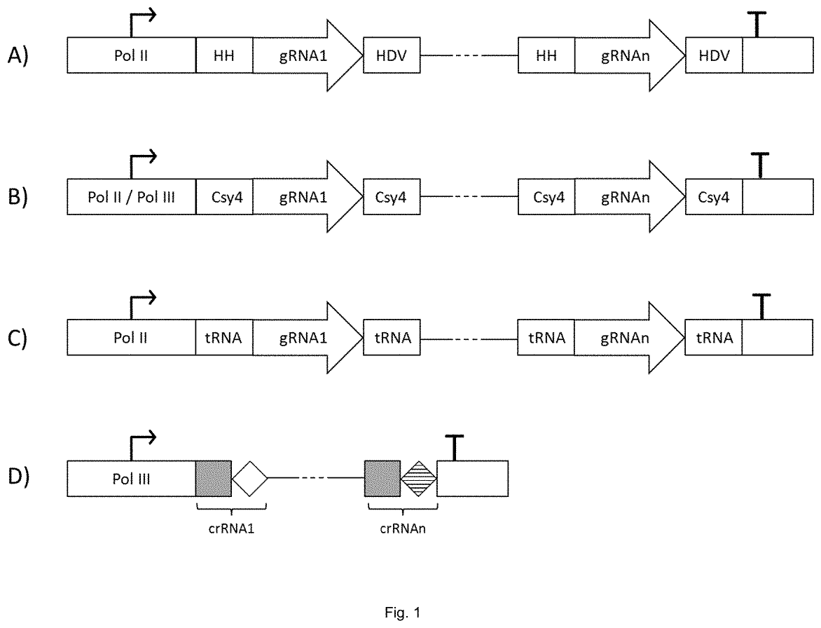

When the RNA processing sequence is a Cas12a Direct Repeat (DR) sequence, it is located on the 5′-part of each guide-RNA molecule or each guide sequence may be flanked by a Cas12a Direct Repeat (DR) sequences (see e.g. FIGS. 1D, 2D, 3 and 4). Cas12a-like enzymes, such as Cas12a, will process the array of at least two functional guide-RNA molecules into separate functional guide-RNA molecules. Cas12a-like enzymes, such as Cas12a (previously Cpf1) are enzymes that have identical features as Cas12a (as described herein above) and are known the person skilled in the art.

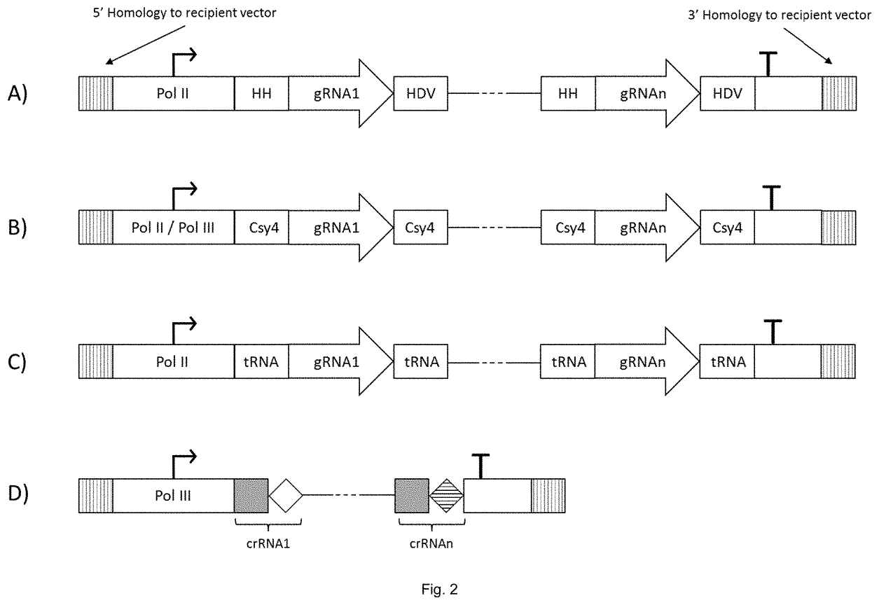

When the RNA processing sequence is a Csy4 recognition sequence, each guide sequence is flanked by Csy4 recognition sequences (see e.g. FIGS. 1B and 2B). Csy4-like enzymes, such Csy4, will process the array of at least two functional guide-RNA molecules into separate functional guide-RNA molecules. The person skilled in the art will comprehend that in order to facilitate proper processing of the array of at least two functional guide-RNA molecules into separate functional guide-RNA molecules, a Csy4-like enzyme needs to be present in the cell. Csy4-like enzymes, such as a Csy4-like enzyme from Pseudomonas aeruginosa, are enzymes that have identical features as Csy4 (as described herein above) and are known the person skilled in the art (see e.g. Haurwitz et al, 2010 and Haurwitz et al, 2012). A Csy4 recognition sequence is known to the person skilled in the art; such as the Csy4 endoribonuclease from Pseudomonas aeruginosa which has a high degree of substrate specificity toward the 28 nucleotides RNA stem-loop sequence 5′-GTTCACTGCCGTATAGGCAGCTAAGAAA-3′ (SEQ ID NO: 67). The person skilled in the art will comprehend that small variations in the Csy4 recognition sequence are allowed as long as the Csy4 endoribonuclease still recognizes and processes the Csy4 recognition sequences in the array.

When the RNA processing sequence is a self-processing ribozyme, each guide sequence is flanked by ribozyme units (see e.g. FIGS. 1B and 2B). Self-processing ribozymes are known to the person skilled in the art and are described herein above. Typically, when a hammerhead ribozyme is used, the hammerhead unit will be located on the 5′-part of each guide-RNA molecule and the ribozyme unit, such as the hepatitis delta ribozyme, will be located on the 3′-part of each guide-RNA molecule. When the RNA processing sequence is a tRNA sequence, each guide sequence is flanked by tRNA sequences. tRNAs are known to the person skilled in the art and are described herein above. Suitable tRNAs for all embodiments of the invention are, especially for S. cerevisiae, are e.g.:

tRNAgly (tGGC) encoding sequence:
(GCGCAAGTGGTTTAGTGGTAAAATCAACGTTGCCATCGTTGGGCCCC
CGGTTCGATTCCGGGCTTGCGCA; SEQ ID NO: 68);
5′ leader sequence for tRNAgly encoding sequence
(AAAATAATAA; SEQ ID NO: 69);
tRNAglu (tTTC) encoding sequence:
(TCCGATATAGTGTAACGGCTATCACATCACGCTTTCACCGTGGAGAC
CGGGGTTCGACTCCCCGTATCGGAG; SEQ ID NO: 70);
5′ leader sequence for tRNAglu encoding sequence
(TTAATTATCA; SEQ ID NO: 71);
tRNAtyr (tAGC) encoding sequence:
(GGGCGTGTGGCGTAGTCGGTAGCGCGCTCCCTTAGCATGGGAGAGGT
CTCCGGTTCGATTCCGGACTCGTCCA; SEQ ID NO: 72);
5′ leader sequence for tRNAtyr encoding sequence
(ACAGAAAATC; SEQ ID NO: 73);
tRNAarg (tCTT) encoding sequence:
(GTTCCGTTGGCGTAATGGTAACGCGTCTCCCTCCTAAGGAGAAGACT
GCGGGTTCGAGTCCCGTACGGAACG; SEQ ID NO: 74);
5′ leader sequence for tRNAarg encoding sequence
(CAACGAAATA; SEQ ID NO: 75);
tRNAasn (tGTT) encoding sequence:
(GACTCCATGGCCAAGTTGGTTAAGGCGTGCGACTGTTAATCGCAAGA
TCGTGAGTTCAACCCTCACTGGGGTCG; SEQ ID NO: 76);
5′ leader sequence for tRNA asn
(AAGATAAAGT; SEQ ID NO: 77);
tRNAile (tAAT) encoding sequence:
(GGTCTCTTGGCCCAGTTGGTTAAGGCACCGTGCTAATAACGCGGGGA
TCAGCGGTTCGATCCCGCTAGAGACCA; SEQ ID NO: 78);
5′ leader sequence for tRNA ile
(ACAAAAGAAT; SEQ ID NO: 79).

A 10 bp leader sequence can be placed 5′ of the tRNA encoding sequence, which exerts strong positive impact on RNAse P processing (Ziehler et al., 2000).

A suitable pair of tRNAs is selected form the group here above, such as tGCC with tTTC.

The person skilled in the art will comprehend that the tRNA encoding sequences here above are S. cerevisiae sequences. For use in other organisms, the person skilled in the art will select the proper counterpart coding sequences for the organism of choice.

The person skilled in the art will comprehend that depending on the processing system used, expression may be controlled differently. When a self-processing ribozyme or a tRNA is used as an RNA processing sequence, expressing is typically performed from an RNA polymerase II promoter. When a Csy4 recognition sequence is used as an RNA processing sequence, expressing is typically performed from an RNA polymerase II promoter or an RNA polymerase III promoter. When a Cas12a Direct Repeat (DR) sequence is used as an RNA processing sequence, expressing is typically performed from an RNA polymerase III promoter.

The person skilled in the art will also comprehend that a Cas12a-like functional guide-RNA molecule is different from a Cas9-like functional guide-RNA molecule. Cas12a-like enzymes are guided by a single crRNA and does not require a tracrRNA. Accordingly, the Cas12a-like functional guide-RNA molecule does not need to comprise a tracrRNA. In contrast, Cas9-like enzymes will need a tracrRNA. Accordingly, the Cas9-like functional guide-RNA molecule will comprise a tracrRNA. The person skilled in the art will be aware of this and knows how to adapt the methods and uses herein according to purpose.

It may be convenient that the assembled double-stranded polynucleotide encoding at least two functional guide-RNA molecules can further assemble or integrate into the linear double-stranded polynucleotide into a double-stranded polynucleotide construct of pre-determined sequence,

Accordingly, there is provided for the use defined herein above, wherein a part at the 5′-end of the double-stranded polynucleotide encoding at least two functional guide-RNA molecules has sequence identity with a part at one terminal part of the linear double-stranded polynucleotide and wherein a part at the 3′-end of the double-stranded polynucleotide encoding the array of at least two functional guide-RNA molecules has sequence identity with the other terminal part of the linear double-stranded polynucleotide, such that the plurality of single-stranded oligonucleotide members, when assembled, can assemble together with the linear double-stranded polynucleotide into the double-stranded polynucleotide construct of pre-determined sequence. The assembly product may be a circular double-stranded polynucleotide construct of pre-determined sequence. The linear double-stranded polynucleotide may be a vector comprising a selectable marker. The parts having sequence identity preferably have at length of at least 5, 10, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. The parts having sequence identity preferably have a length of 5, 10, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. The degree of complementarity, when optimally aligned using a suitable alignment algorithm, is preferably higher than 50%, 60%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity. The sequence identity may be 100%.

There is provided for the use defined herein above, wherein the oligonucleotide members comprise overlapping portions at least 10 bases each, such that they are capable of assembly within a cell into a double-stranded polynucleotide encoding an array of at least two functional guide-RNA molecules. The person skilled in the art will comprehend that the oligonucleotide members should comprise sufficient overlap to be capable of assembly under physiological conditions in a cell. The overlapping portions may be different for individual members and may e.g. be 10, 11, 12, 13, 14, 15, 16, 17, 18, 19, 20, 21, 22, 23, 24, 25, 26, 27, 28, 29, 30 nucleotides. The degree of complementarity of the overlapping portions, when optimally aligned using a suitable alignment algorithm, is preferably higher than 50%, 60%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity. The sequence identity may be 100%. The portions of the oligonucleotide members may contain one or more gaps of e.g. 1 or 2 nucleotides, as long as long as the members still comprise sufficient overlap to be capable of assembly under physiological conditions in a cell. Gaps in nucleotide assembly are known to the person skilled in the art, see e.g. Gibson et al, 2009.

There is provided for the use defined herein above, wherein the double-stranded polynucleotide encodes an array of three, four, five, six or more functional guide-RNA molecules.

When used for multiplex genome or gene editing, it is convenient that the functional guide-RNA molecules are different (distinct), e.g. are directed to different target-sequences in the target-polynucleotide such as a target genome. Accordingly, there is provided for the use as defined herein above, wherein the functional guide-RNA molecules are distinct functional guide-RNA molecules. As e.g. evident from the figures and examples herein, the plurality of single-stranded oligonucleotide members may comprise at least three, four, five, six or more single-stranded oligonucleotide members.

The person skilled in the art comprehends that the array of two or more functional guide-RNA molecules needs control sequences such as a promoter and terminator for expression of the array. Accordingly, the linear double-stranded polynucleotide may comprise a promoter, or a part thereof, that, after assembly, is operably linked to the polynucleotide encoding the array of at least two functional guide-RNA molecules. Such promoter may be as desired of required e.g. an RNA polymerase II promoter or an RNA polymerase III promoter.

Accordingly, the linear double-stranded polynucleotide may comprise a terminator that, after assembly, is operably linked to the polynucleotide encoding the array of at least two functional guide-RNA molecules. Alternatively, the terminator may be present and operably linked to the polynucleotide encoding the array of at least two functional guide-RNA molecules.

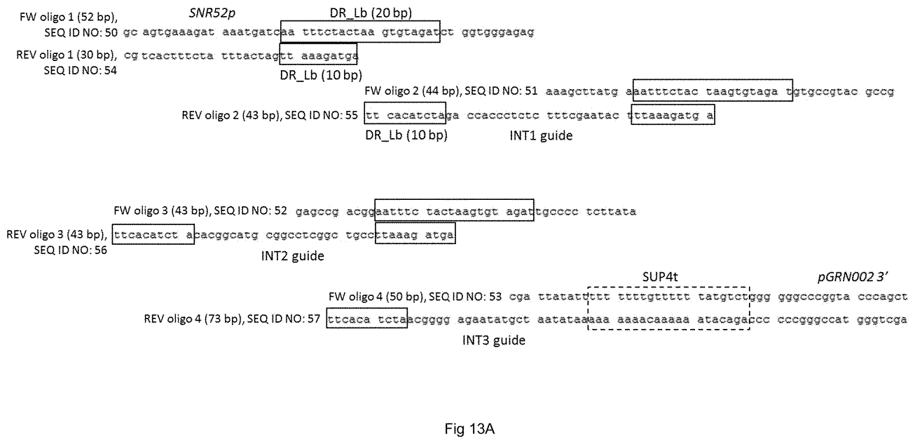

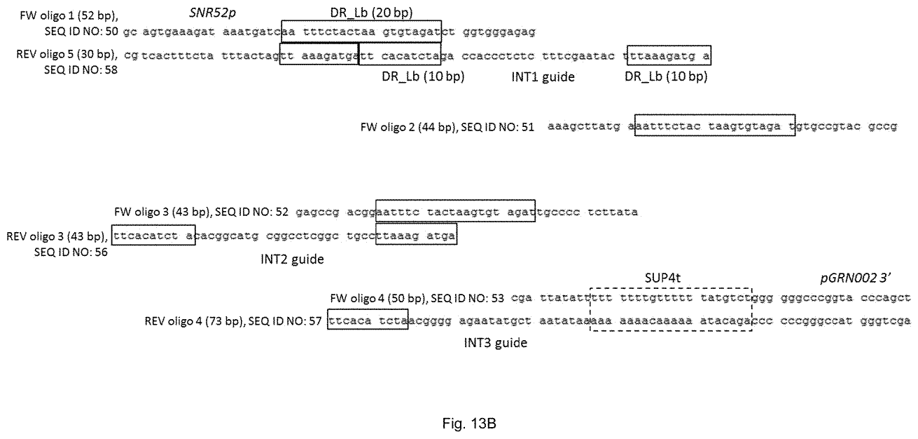

There is provided for several ways to obtain the assembled double-stranded polynucleotide encoding an array of two or more functional guide-RNA molecules, see e.g. FIGS. 12 and 13. There is provided a convenient way wherein changing one of the genomic targets by another one requires changing just three oligonucleotides of the plurality of single-stranded oligonucleotide members. In this provision, two reverse oligonucleotide members and one forward oligonucleotide member are used to obtain a functional guide-RNA molecule or wherein two forward oligonucleotide members and one reverse oligonucleotide member are used for a functional guide-RNA molecule, see e.g. FIGS. 12 and 13.

In a second aspect, there is provided for a method for assembly within a cell, of a double-stranded polynucleotide construct of pre-determined sequence comprising contacting a cell with a plurality of single-stranded oligonucleotide members and a linear double-stranded polynucleotide member such that these are introduced into the cell, wherein the members of the plurality of single-stranded oligonucleotides are capable of assembly within a cell into a double-stranded polynucleotide encoding an array of at least two functional guide-RNA molecules, wherein each guide-RNA molecule comprises at least a guide sequence and an RNA processing sequence.

In this aspect, the features of the embodiments are preferably those of the corresponding embodiments of the first aspect.

In this aspect, there is provided for a method, wherein the RNA processing sequence is a Cas12a Direct Repeat (DR) sequence, a Csy4 recognition sequence, a self-processing ribozyme or a tRNA.

In this aspect, there is provided for a method, wherein the RNA processing sequence is a Cas12a Direct Repeat (DR) sequence and is located on the 5′-part of each guide-RNA molecule, or wherein each guide sequence is flanked by Cas12a Direct Repeat (DR) sequences.

In this aspect, there is provided for a method, wherein the RNA processing sequence is a Csy4 recognition sequence, and wherein each guide sequence is flanked by Csy4 recognition sequences.

In this aspect, there is provided for a method, wherein the RNA processing sequence is a self-processing ribozyme and each guide sequence is flanked by ribozyme units.

In this aspect, there is provided for a method, wherein the RNA processing sequence is a tRNA and each guide sequence is flanked by tRNA's.

In this aspect, there is provided for a method, wherein a part at the 5′-end of the double-stranded polynucleotide encoding at least two functional guide-RNA molecules has sequence identity with a part at one terminal part of the linear double-stranded polynucleotide and wherein a part at the 3′-end of the double-stranded polynucleotide encoding the array of at least two functional guide-RNA molecules has sequence identity with the other terminal part of the linear double-stranded polynucleotide, such that the plurality of single-stranded oligonucleotide members, when assembled, can assemble together with the linear double-stranded polynucleotide into the double-stranded polynucleotide construct.

In this aspect, there is provided for a method, wherein the oligonucleotide members comprise overlapping portions at least 10 bases each, such that they are capable of assembly within a cell into a double-stranded polynucleotide encoding an array of at least two functional guide-RNA molecules.

In this aspect, there is provided for a method, wherein the double-stranded polynucleotide encodes an array of three, four, five, six or more functional guide-RNA molecules.

In this aspect, there is provided for a method, wherein the functional guide-RNA molecules are distinct functional guide-RNA molecules.

In this aspect, there is provided for a method, wherein the plurality of single-stranded oligonucleotide members comprises at least three, four, five, six or more members.

In this aspect, there is provided for a method, wherein the portions of the oligonucleotide members contain one or more gaps.

In this aspect, there is provided for a method, wherein the linear double-stranded polynucleotide is a vector comprising a selectable marker.

In this aspect, there is provided for a method, wherein the assembly results in a circular double-stranded polynucleotide construct of pre-determined sequence.

In this aspect, there is provided for a method, wherein the linear double-stranded polynucleotide comprises a promoter, or a part thereof, that, after assembly, is operably linked to the polynucleotide encoding the array of at least two functional guide-RNA molecules.

In this aspect, there is provided for a method, wherein the linear double-stranded polynucleotide comprises a terminator that, after assembly, is operably linked to the polynucleotide encoding the array of at least two functional guide-RNA molecules.

In this aspect, there is provided for a method, wherein the polynucleotide encoding the array of at least two functional guide-RNA molecules comprises a terminator that is operably linked to it.

In this aspect, there is provided for a method, wherein two reverse oligonucleotide members and one forward oligonucleotide member are used for a functional guide-RNA molecule or wherein two forward oligonucleotide members and one reverse oligonucleotide member are used for a functional guide-RNA molecule.

In a third aspect, there is provided for, a method for expression within a cell of at least two functional guide-RNA molecules, comprising contacting a cell with a plurality of single-stranded oligonucleotide members and a linear double-stranded polynucleotide member such that these are introduced into the cell, wherein the members of the plurality of single-stranded oligonucleotides are capable of assembly within a cell into a double-stranded polynucleotide encoding an array of at least two functional guide-RNA molecules, wherein each guide-RNA molecule comprises at least a guide sequence and an RNA processing sequence.

In this aspect, the features of the embodiments are preferably those of the corresponding embodiments of the first and second aspect. The cell or, interchangeably host cell, is defined in the section “Definitions”.

In this aspect, there is provided for a method, wherein the RNA processing sequence is a Cas12a Direct Repeat (DR) sequence, a Csy4 recognition sequence, a self-processing ribozyme or a tRNA.

In this aspect, there is provided for a method, wherein the RNA processing sequence is a Cas12a Direct Repeat (DR) sequence and is located on the 5′-part of each guide-RNA molecule, or wherein each guide sequence is flanked by Cas12a Direct Repeat (DR) sequences.

In this aspect, there is provided for a method, wherein the RNA processing sequence is a Csy4 recognition sequence, and wherein each guide sequence is flanked by Csy4 recognition sequences.

In this aspect, there is provided for a method, wherein the RNA processing sequence is a self-processing ribozyme and each guide sequence is flanked by ribozyme units.

In this aspect, there is provided for a method, wherein the RNA processing sequence is a tRNA and each guide sequence is flanked by tRNA's.

In this aspect, there is provided for a method, wherein the cell expresses a functional Cas12a-like enzyme, a functional Csy4 and/or a functional Cas9-like enzyme or wherein in the cell a functional Cas12a-like enzyme, a functional Csy4 and/or a functional Cas9-like enzyme is present. Methods to express such enzyme or to introduce such enzyme into the cell are known to the person skilled in the art; several of such methods are listed in the examples herein.

In this aspect, there is provided for a method, wherein the functional Cas12a-like enzyme, the functional Csy4 and/or the functional Cas9-like enzyme are introduced into the cell together with the plurality of single-stranded oligonucleotide members and the linear double-stranded polynucleotide or wherein a vector capable of expressing a functional Cas12a-like enzyme, the functional Csy4 and/or the functional Cas9-like enzyme are introduced into the cell together with the plurality of single-stranded oligonucleotide members and the linear double-stranded polynucleotide.

In this aspect, there is provided for a method, wherein a part at the 5′-end of the double-stranded polynucleotide encoding at least two functional guide-RNA molecules has sequence identity with a part at one terminal part of the linear double-stranded polynucleotide and wherein a part at the 3′-end of the double-stranded polynucleotide encoding the array of at least two functional guide-RNA molecules has sequence identity with the other terminal part of the linear double-stranded polynucleotide, such that the plurality of single-stranded oligonucleotide members, when assembled, can assemble together with the linear double-stranded polynucleotide into the double-stranded polynucleotide construct.

In this aspect, there is provided for a method, wherein the oligonucleotide members comprise overlapping portions at least 10 bases each, such that they are capable of assembly within a cell into a double-stranded polynucleotide encoding an array of at least two functional guide-RNA molecules.

In this aspect, there is provided for a method, wherein the double-stranded polynucleotide encodes an array of three, four, five, six or more functional guide-RNA molecules.

In this aspect, there is provided for a method, wherein the functional guide-RNA molecules are distinct functional guide-RNA molecules.

In this aspect, there is provided for a method, wherein the plurality of single-stranded oligonucleotide members comprises at least three, four, five, six or more members.

In this aspect, there is provided for a method, wherein the portions of the oligonucleotide members contain one or more gaps.

In this aspect, there is provided for a method, wherein the linear double-stranded polynucleotide is a vector comprising a selectable marker.

In this aspect, there is provided for a method, wherein the assembly results in a circular double-stranded polynucleotide construct of pre-determined sequence.

In this aspect, there is provided for a method, wherein the linear double-stranded polynucleotide comprises a promoter, or a part thereof, that, after assembly, is operably linked to the polynucleotide encoding the array of at least two functional guide-RNA molecules.

In this aspect, there is provided for a method, wherein the linear double-stranded polynucleotide comprises a terminator that, after assembly, is operably linked to the polynucleotide encoding the array of at least two functional guide-RNA molecules.

In this aspect, there is provided for a method, wherein the polynucleotide encoding the array of at least two functional guide-RNA molecules comprises a terminator that is operably linked to it.

In this aspect, there is provided for a method, wherein two reverse oligonucleotide members and one forward oligonucleotide member are used for a functional guide-RNA molecule or wherein two forward oligonucleotide members and one reverse oligonucleotide member are used for a functional guide-RNA molecule.

In a fourth aspect, there is provided for a method for gene editing, comprising contacting a cell with a plurality of single-stranded oligonucleotide members and a linear double-stranded polynucleotide member such that these are introduced into the cell, wherein the members of the plurality of single-stranded oligonucleotides are capable of assembly within a cell into a double-stranded polynucleotide encoding an array of at least two functional guide-RNA molecules, wherein each guide-RNA molecule comprises at least a guide sequence and an RNA processing sequence.

In this aspect, the features of the embodiments are preferably those of the corresponding embodiments of the first, second and third aspect. The cell or, interchangeably host cell, is defined in the section “Definitions”.

In this aspect, there is provided for a method, wherein the RNA processing sequence is a Cas12a Direct Repeat (DR) sequence, a Csy4 recognition sequence, a self-processing ribozyme or a tRNA.

In this aspect, there is provided for a method, wherein the RNA processing sequence is a Cas12a Direct Repeat (DR) sequence and is located on the 5′-part of each guide-RNA molecule, or wherein each guide sequence is flanked by Cas12a Direct Repeat (DR) sequences.

In this aspect, there is provided for a method, wherein the RNA processing sequence is a Csy4 recognition sequence, and wherein each guide sequence is flanked by Csy4 recognition sequences.

In this aspect, there is provided for a method, wherein the RNA processing sequence is a self-processing ribozyme and each guide sequence is flanked by ribozyme units.

In this aspect, there is provided for a method, wherein the RNA processing sequence is a tRNA and each guide sequence is flanked by tRNA's.

In this aspect, there is provided for a method, wherein the cell expresses a functional Cas12a-like enzyme, a functional Csy4 and/or a functional Cas9-like enzyme or wherein in the cell a functional

Cas12a-like enzyme, a functional Csy4 and/or a functional Cas9-like enzyme is present. In this aspect, there is provided for a method, wherein the functional Cas12a-like enzyme, the functional Csy4 and/or the functional Cas9 enzyme are introduced into the cell together with the plurality of single-stranded oligonucleotide members and the linear double-stranded polynucleotide or wherein a vector capable of expressing a functional Cas12a-like enzyme, the functional Csy4 and/or the functional Cas9-like enzyme are introduced into the cell together with the plurality of single-stranded oligonucleotide members and the linear double-stranded polynucleotide.

In this aspect, there is provided for a method, wherein a part at the 5′-end of the double-stranded polynucleotide encoding at least two functional guide-RNA molecules has sequence identity with a part at one terminal part of the linear double-stranded polynucleotide and wherein a part at the 3′-end of the double-stranded polynucleotide encoding the array of at least two functional guide-RNA molecules has sequence identity with the other terminal part of the linear double-stranded polynucleotide, such that the plurality of single-stranded oligonucleotide members, when assembled, can assemble together with the linear double-stranded polynucleotide into the double-stranded polynucleotide construct.

In this aspect, there is provided for a method, wherein the oligonucleotide members comprise overlapping portions at least 10 bases each, such that they are capable of assembly within a cell into a double-stranded polynucleotide encoding an array of at least two functional guide-RNA molecules.

In this aspect, there is provided for a method, wherein the double-stranded polynucleotide encodes an array of three, four, five, six or more functional guide-RNA molecules

In this aspect, there is provided for a method, wherein the functional guide-RNA molecules are distinct functional guide-RNA molecules.

In this aspect, there is provided for a method, wherein the plurality of single-stranded oligonucleotide members comprises at least three, four, five, six or more members.

In this aspect, there is provided for a method, wherein the portions of the oligonucleotide members contain one or more gaps.

In this aspect, there is provided for a method, wherein the linear double-stranded polynucleotide is a vector comprising a selectable marker.

In this aspect, there is provided for a method, wherein the assembly results in a circular double-stranded polynucleotide construct of pre-determined sequence.

In this aspect, there is provided for a method, wherein the linear double-stranded polynucleotide comprises a promoter, or a part thereof, that, after assembly, is operably linked to the polynucleotide encoding the array of at least two functional guide-RNA molecules.

In this aspect, there is provided for a method, wherein the linear double-stranded polynucleotide comprises a terminator that, after assembly, is operably linked to the polynucleotide encoding the array of at least two functional guide-RNA molecules.

In this aspect, there is provided for a method, wherein the polynucleotide encoding the array of at least two functional guide-RNA molecules comprises a terminator that is operably linked to it.

In this aspect, there is provided for a method, wherein two reverse oligonucleotide members and one forward oligonucleotide member are used for a functional guide-RNA molecule or wherein two forward oligonucleotide members and one reverse oligonucleotide member are used for a functional guide-RNA molecule.

CRISPR genome engineering and CRISPR multiplex genome engineering have numerous applications known to the person skilled in the art, such as genome editing and gene regulation applications. Several of these techniques involve a heterologous (donor) polynucleotide that integrates into the genome of the cell in the proximity of the break mediated by a functional genome editing system.

Accordingly, in this aspect, there is provided for a method, wherein in the cell a heterologous polynucleotide is present that integrates into the genome of the cell in the proximity of the break mediated by the functional complex of Cas12a-like enzyme or a Cas9-like enzyme and one of the at least two functional guide-RNA molecules. The break in the genome can be a double-stranded or a single-stranded break.

In a fifth aspect, there is provided for a double-stranded polynucleotide encoding an array of at least two guide-RNA molecules obtainable or obtained by a method according to any one of the methods and embodiments of the second, third and fourth aspect, wherein each guide-RNA molecule comprises at least a guide sequence and an RNA processing sequence.

In this aspect, the features of the embodiments are preferably those of the corresponding embodiments of the first, second, third and fourth aspect. The a double-stranded polynucleotide encoding an array of at least two guide-RNA molecules according to this aspect may be comprised in the double-stranded polynucleotide construct.

The double-stranded polynucleotide or double-stranded polynucleotide construct according to this aspect of the invention can be isolated from the cell using methods known to the person skilled in the art, such as PCR or by e.g. plasmid rescue when the double-stranded polynucleotide is a plasmid or vectors.

In this aspect, there is provided for a double-stranded polynucleotide, wherein the RNA processing sequence is a Cas12a Direct Repeat (DR) sequence, a Csy4 recognition sequence, a self-processing ribozyme or a tRNA.

In this aspect, there is provided for a double-stranded polynucleotide, wherein the RNA processing sequence is a Cas12a Direct Repeat (DR) sequence and is located on the 5′-part of each guide-RNA molecule, or wherein each guide sequence is flanked by Cas12a Direct Repeat (DR) sequences.

In this aspect, there is provided for a double-stranded polynucleotide, wherein the RNA processing sequence is a Csy4 recognition sequence, and wherein each guide sequence is flanked by Csy4 recognition sequences.

In this aspect, there is provided for a double-stranded polynucleotide, wherein the RNA processing sequence is a self-processing ribozyme and each guide sequence is flanked by ribozyme units.

In this aspect, there is provided for a double-stranded polynucleotide, wherein the RNA processing sequence is a tRNA and each guide sequence is flanked by tRNA's.

In a sixth aspect, there is provided a cell obtainable by or obtained by a method according to any one of the methods and embodiments of the second, third and fourth aspect.

In this aspect, the features of the embodiments are preferably those of the corresponding embodiments of the first, second, third and fourth aspect. The cell or, interchangeably host cell, is defined in the section “Definitions”.

In a seventh aspect, there is provided for a method for the production of a double-stranded polynucleotide encoding an array of at least two guide-RNA molecules obtainable or obtained by a method according to any one of the methods and embodiments of the second, third and fourth aspect, wherein each guide-RNA molecule comprises at least a guide sequence and an RNA processing sequence, said method comprising, performing a method according to any one of the methods and embodiments of the second, third and fourth aspect and subsequently isolating the double-stranded polynucleotide from the cell.

In this aspect, the features of the embodiments are preferably those of the corresponding embodiments of the first, second, third and fourth aspect. The a double-stranded polynucleotide encoding an array of at least two guide-RNA molecules according to this aspect may be comprised in the double-stranded polynucleotide construct.

The double-stranded polynucleotide or double-stranded polynucleotide construct according to this aspect of the invention can be isolated from the cell using methods known to the person skilled in the art, such as PCR or by e.g. plasmid rescue when the double-stranded polynucleotide is a plasmid or vectors.

In this aspect, there is provided for a method wherein the RNA processing sequence is a Cas12a Direct Repeat (DR) sequence, a Csy4 recognition sequence, a self-processing ribozyme or a tRNA.

In this aspect, there is provided for a method wherein the RNA processing sequence is a Cas12a Direct Repeat (DR) sequence and is located on the 5′-part of each guide-RNA molecule, or wherein each guide sequence is flanked by Cas12a Direct Repeat (DR) sequences.

In this aspect, there is provided for a method wherein the RNA processing sequence is a Csy4 recognition sequence, and wherein each guide sequence is flanked by Csy4 recognition sequences.

In this aspect, there is provided for a method wherein the RNA processing sequence is a self-processing ribozyme and each guide sequence is flanked by ribozyme units.

In this aspect, there is provided for a method wherein the RNA processing sequence is a tRNA and each guide sequence is flanked by tRNA's.

In this aspect, there is provided for a method wherein the double-stranded polynucleotide is amplified from the cell.

In this aspect, there is provided for a method wherein the double-stranded polynucleotide construct comprising the double stranded polynucleotide is rescued from the cell.

In an eight aspect, there is provided for a method for the production of a compound of interest comprising, culturing a cell according to the sixth aspect, said cell comprising a polynucleotide encoding a compound of interest, under conditions conducive to the production of the compound of interest, and optionally isolating and/or purifying the compound of interest.

In this aspect, the features of the embodiments are preferably those of the corresponding embodiments of the first, second, third, fourth, fifth, sixth and sevenths aspect.

The cell or, interchangeably host cell, is defined in the section “Definitions”. The compound of interest is also defined the section “Definitions”.

### EMBODIMENTS

The following embodiments are provided; the features in these embodiments are preferably those as defined previously herein.

1. Use of a plurality of single-stranded oligonucleotide members and a linear double-stranded polynucleotide member in the assembly within a cell of a double-stranded polynucleotide construct of pre-determined sequence, wherein the members of the plurality of single-stranded oligonucleotides are capable of assembly within a cell into a double-stranded polynucleotide encoding an array of at least two functional guide-RNA molecules, wherein each guide-RNA molecule comprises at least a guide sequence and an RNA processing sequence.

2. Use according to embodiment 1, wherein the RNA processing sequence is a Cas12a Direct Repeat (DR) sequence, a Csy4 recognition sequence, a self-processing ribozyme or a tRNA sequence.

3. Use according to embodiment 1, wherein the RNA processing sequence is a Cas12a Direct Repeat (DR) sequence and is located on the 5′-part of each guide-RNA molecule, or wherein each guide sequence is flanked by a Cas12a Direct Repeat (DR) sequences.

4. Use according to embodiment 1, wherein the RNA processing sequence is a Csy4 recognition sequence, and wherein each guide sequence is flanked by Csy4 recognition sequences.

5. Use according to embodiment 1, wherein the RNA processing sequence is a self-processing ribozyme and each guide sequence is flanked by ribozyme units.

6. Use according to embodiment 1, wherein the RNA processing sequence is a tRNA sequence and each guide sequence is flanked by tRNA sequences.

7. Use according to any one of the preceding embodiments, wherein a part at the 5′-end of the double-stranded polynucleotide encoding at least two functional guide-RNA molecules has sequence identity with a part at one terminal part of the linear double-stranded polynucleotide and wherein a part at the 3′-end of the double-stranded polynucleotide encoding the array of at least two functional guide-RNA molecules has sequence identity with the other terminal part of the linear double-stranded polynucleotide, such that the plurality of single-stranded oligonucleotide members, when assembled, can assemble together with the linear double-stranded polynucleotide into the double-stranded polynucleotide construct.

8. Use according to any one of the preceding embodiments, wherein the oligonucleotide members comprise overlapping portions at least 10 bases each, such that they are capable of assembly within a cell into a double-stranded polynucleotide encoding an array of at least two functional guide-RNA molecules.

9. Use according to any one of the preceding embodiments, wherein the double-stranded polynucleotide encodes an array of three, four, five, six or more functional guide-RNA molecules.

10. Use according to any one of the preceding embodiments, wherein the functional guide-RNA molecules are distinct functional guide-RNA molecules.

11. Use according to any one of the preceding embodiments, wherein the plurality of single-stranded oligonucleotide members comprises at least three, four, five, six or more members.

12. Use according to any one of the preceding embodiments, wherein the portions of the oligonucleotide members contain one or more gaps.

13. Use according to any one of the preceding embodiments, wherein the linear double-stranded polynucleotide is a vector comprising a selectable marker.

14. Use according to any one of the preceding embodiments, wherein the assembly results in a circular double-stranded polynucleotide construct of pre-determined sequence.

15. Use according to any one of the preceding embodiments, wherein the linear double-stranded polynucleotide comprises a promoter, or a part thereof, that, after assembly, is operably linked to the polynucleotide encoding the array of at least two functional guide-RNA molecules.

16. Use according to any one of the preceding embodiments, wherein the linear double-stranded polynucleotide comprises a terminator that, after assembly, is operably linked to the polynucleotide encoding the array of at least two functional guide-RNA molecules.

17. Use according to any one of embodiments 1 to 12, wherein the polynucleotide encoding the array of at least two functional guide-RNA molecules comprises a terminator that is operably linked to it.

18. Use according to any one of the preceding embodiments, wherein two reverse oligonucleotide members and one forward oligonucleotide member are used to obtain a functional guide-RNA molecule or wherein two forward oligonucleotide members and one reverse oligonucleotide member are used for a functional guide-RNA molecule.

19. A method for assembly within a cell, of a double-stranded polynucleotide construct of pre-determined sequence comprising contacting a cell with a plurality of single-stranded oligonucleotide members and a linear double-stranded polynucleotide member such that these are introduced into the cell, wherein the members of the plurality of single-stranded oligonucleotides are capable of assembly within a cell into a double-stranded polynucleotide encoding an array of at least two functional guide-RNA molecules, wherein each guide-RNA molecule comprises at least a guide sequence and an RNA processing sequence.

20. A method according to embodiment 19, wherein the RNA processing sequence is a Cas12a Direct Repeat (DR) sequence, a Csy4 recognition sequence, a self-processing ribozyme or a tRNA.

21. A method according to embodiment 19, wherein the RNA processing sequence is a Cas12a Direct Repeat (DR) sequence and is located on the 5′-part of each guide-RNA molecule, or wherein each guide sequence is flanked by Cas12a Direct Repeat (DR) sequences.

22. A method according to embodiment 19, wherein the RNA processing sequence is a Csy4 recognition sequence, and wherein each guide sequence is flanked by Csy4 recognition sequences.

23. A method according to embodiment 19, wherein the RNA processing sequence is a self-processing ribozyme and each guide sequence is flanked by ribozyme units.

24. A method according to embodiment 19, wherein the RNA processing sequence is a tRNA and each guide sequence is flanked by tRNA's.

25. A method according to any one of embodiments 19 to 24, wherein a part at the 5′-end of the double-stranded polynucleotide encoding at least two functional guide-RNA molecules has sequence identity with a part at one terminal part of the linear double-stranded polynucleotide and wherein a part at the 3′-end of the double-stranded polynucleotide encoding the array of at least two functional guide-RNA molecules has sequence identity with the other terminal part of the linear double-stranded polynucleotide, such that the plurality of single-stranded oligonucleotide members, when assembled, can assemble together with the linear double-stranded polynucleotide into the double-stranded polynucleotide construct.

26. A method according to any one of embodiments 19 to 25, wherein the oligonucleotide members comprise overlapping portions at least 10 bases each, such that they are capable of assembly within a cell into a double-stranded polynucleotide encoding an array of at least two functional guide-RNA molecules.

27. A method according to any one of embodiments 19 to 26, wherein the double-stranded polynucleotide encodes an array of three, four, five, six or more functional guide-RNA molecules.

28. A method according to any one of embodiments 19 to 27, wherein the functional guide-RNA molecules are distinct functional guide-RNA molecules.

29. Use according to any one of embodiments 19 to 28, wherein the plurality of single-stranded oligonucleotide members comprises at least three, four, five, six or more members.

30. A method according to any one of embodiments 19 to 29, wherein the portions of the oligonucleotide members contain one or more gaps.

31. A method according to any one of embodiments 19 to 30, wherein the linear double-stranded polynucleotide is a vector comprising a selectable marker.

32. A method according to any one of embodiments 19 to 31, wherein the assembly results in a circular double-stranded polynucleotide construct of pre-determined sequence.

33. A method according to any one of embodiments 19 to 32, wherein the linear double-stranded polynucleotide comprises a promoter, or a part thereof, that, after assembly, is operably linked to the polynucleotide encoding the array of at least two functional guide-RNA molecules.

34. A method according to any one of embodiments 19 to 33, wherein the linear double-stranded polynucleotide comprises a terminator that, after assembly, is operably linked to the polynucleotide encoding the array of at least two functional guide-RNA molecules.

35. A method according to any one of embodiments 19 to 34, wherein the polynucleotide encoding the array of at least two functional guide-RNA molecules comprises a terminator that is operably linked to it.

36. A method according to any one of embodiments 19 to 35, wherein two reverse oligonucleotide members and one forward oligonucleotide member are used for a functional guide-RNA molecule or wherein two forward oligonucleotide members and one reverse oligonucleotide member are used for a functional guide-RNA molecule.

37. A method for expression, within a cell of at least two functional guide-RNA molecules, comprising contacting a cell with a plurality of single-stranded oligonucleotide members and a linear double-stranded polynucleotide member such that these are introduced into the cell, wherein the members of the plurality of single-stranded oligonucleotides are capable of assembly within a cell into a double-stranded polynucleotide encoding an array of at least two functional guide-RNA molecules, wherein each guide-RNA molecule comprises at least a guide sequence and an RNA processing sequence.

38. A method according to embodiment 37, wherein the RNA processing sequence is a Cas12a Direct Repeat (DR) sequence, a Csy4 recognition sequence, a self-processing ribozyme or a tRNA.

39. A method according to embodiment 37, wherein the RNA processing sequence is a Cas12a Direct Repeat (DR) sequence and is located on the 5′-part of each guide-RNA molecule, or wherein each guide sequence is flanked by Cas12a Direct Repeat (DR) sequences.

40. A method according to embodiment 37, wherein the RNA processing sequence is a Csy4 recognition sequence, and wherein each guide sequence is flanked by Csy4 recognition sequences.

41. A method according to embodiment 37, wherein the RNA processing sequence is a self-processing ribozyme and each guide sequence is flanked by ribozyme units.

42. A method according to embodiment 37, wherein the RNA processing sequence is a tRNA and each guide sequence is flanked by tRNA's.

43. A method according to any of embodiments 37 to 42, wherein the cell expresses a functional Cas12a-like enzyme, a functional Csy4 and/or a functional Cas9-like enzyme or wherein in the cell a functional Cas12a-like enzyme, a functional Csy4 and/or a functional Cas9-like enzyme is present.

44. A method according to any one of embodiments 37 to 43, wherein the functional Cas12a, the functional Csy4 and/or the functional Cas9-like enzyme are introduced into the cell together with the plurality of single-stranded oligonucleotide members and the linear double-stranded polynucleotide or wherein a vector capable of expressing a functional Cas12a, the functional Csy4 and/or the functional Cas9-like enzyme are introduced into the cell together with the plurality of single-stranded oligonucleotide members and the linear double-stranded polynucleotide.

45. A method according to any one of embodiments 37 to 44, wherein a part at the 5′-end of the double-stranded polynucleotide encoding at least two functional guide-RNA molecules has sequence identity with a part at one terminal part of the linear double-stranded polynucleotide and wherein a part at the 3′-end of the double-stranded polynucleotide encoding the array of at least two functional guide-RNA molecules has sequence identity with the other terminal part of the linear double-stranded polynucleotide, such that the plurality of single-stranded oligonucleotide members, when assembled, can assemble together with the linear double-stranded polynucleotide into the double-stranded polynucleotide construct.

46. A method according to any one of embodiments 37 to 45, wherein the oligonucleotide members comprise overlapping portions at least 10 bases each, such that they are capable of assembly within a cell into a double-stranded polynucleotide encoding an array of at least two functional guide-RNA molecules.

47. A method according to any one of embodiments 37 to 46, wherein the double-stranded polynucleotide encodes an array of three, four, five, six or more functional guide-RNA molecules.

48. A method according to any one of embodiments 37 to 47, wherein the functional guide-RNA molecules are distinct functional guide-RNA molecules.

49. Use according to any one of embodiments 37 to 48, wherein the plurality of single-stranded oligonucleotide members comprises at least three, four, five, six or more members.

50. A method according to any one of embodiments 37 to 49, wherein the portions of the oligonucleotide members contain one or more gaps.

51. A method according to any one of embodiments 37 to 50, wherein the linear double-stranded polynucleotide is a vector comprising a selectable marker.

52. A method according to any one of embodiments 37 to 51, wherein the assembly results in a circular double-stranded polynucleotide construct of pre-determined sequence.

53. A method according to any one of embodiments 37 to 52, wherein the linear double-stranded polynucleotide comprises a promoter, or a part thereof, that, after assembly, is operably linked to the polynucleotide encoding the array of at least two functional guide-RNA molecules.

54. A method according to any one of embodiments 37 to 53, wherein the linear double-stranded polynucleotide comprises a terminator that, after assembly, is operably linked to the polynucleotide encoding the array of at least two functional guide-RNA molecules.

55. A method according to any one of embodiments 37 to 54, wherein the polynucleotide encoding the array of at least two functional guide-RNA molecules comprises a terminator that is operably linked to it.

56. A method according to any one of embodiments 37 to 55, wherein two reverse oligonucleotide members and one forward oligonucleotide member are used for a functional guide-RNA molecule or wherein two forward oligonucleotide members and one reverse oligonucleotide member are used for a functional guide-RNA molecule.

57. A method for gene editing, comprising contacting a cell with a plurality of single-stranded oligonucleotide members and a linear double-stranded polynucleotide member such that these are introduced into the cell, wherein the members of the plurality of single-stranded oligonucleotides are capable of assembly within a cell into a double-stranded polynucleotide encoding an array of at least two functional guide-RNA molecules, wherein each guide-RNA molecule comprises at least a guide sequence and an RNA processing sequence.

58. A method according to embodiment 57, wherein the RNA processing sequence is a Cas12a Direct Repeat (DR) sequence, a Csy4 recognition sequence, a self-processing ribozyme or a tRNA.

59. A method according to embodiment 57, wherein the RNA processing sequence is a Cas12a Direct Repeat (DR) sequence and is located on the 5′-part of each guide-RNA molecule, or wherein each guide sequence is flanked by Cas12a Direct Repeat (DR) sequences.

60. A method according to embodiment 57, wherein the RNA processing sequence is a Csy4 recognition sequence, and wherein each guide sequence is flanked by Csy4 recognition sequences.

61. A method according to embodiment 57, wherein the RNA processing sequence is a self-processing ribozyme and each guide sequence is flanked by ribozyme units.

62. A method according to embodiment 57, wherein the RNA processing sequence is a tRNA and each guide sequence is flanked by tRNA's.

63. A method according to any of embodiments 57 to 62, wherein the cell expresses a functional Cas12a-like enzyme, a functional Csy4 and/or a functional Cas9-like enzyme or wherein in the cell a functional Cas12a-like enzyme, a functional Csy4 and/or a functional Cas9-like enzyme is present.

64. A method according to any one of embodiments 57 to 63, wherein the functional Cas12a-like enzyme, the functional Csy4 and/or the functional Cas9-like enzyme are introduced into the cell together with the plurality of single-stranded oligonucleotide members and the linear double-stranded polynucleotide or wherein a vector capable of expressing a functional Cas12a-like enzyme, the functional Csy4 and/or the functional Cas9-like enzyme are introduced into the cell together with the plurality of single-stranded oligonucleotide members and the linear double-stranded polynucleotide.

65. A method according to any one of embodiments 57 to 64, wherein a part at the 5′-end of the double-stranded polynucleotide encoding at least two functional guide-RNA molecules has sequence identity with a part at one terminal part of the linear double-stranded polynucleotide and wherein a part at the 3′-end of the double-stranded polynucleotide encoding the array of at least two functional guide-RNA molecules has sequence identity with the other terminal part of the linear double-stranded polynucleotide, such that the plurality of single-stranded oligonucleotide members, when assembled, can assemble together with the linear double-stranded polynucleotide into the double-stranded polynucleotide construct.

66. A method according to any one of embodiments 57 to 65, wherein the oligonucleotide members comprise overlapping portions at least 10 bases each, such that they are capable of assembly within a cell into a double-stranded polynucleotide encoding an array of at least two functional guide-RNA molecules.

67. A method according to any one of embodiments 57 to 66, wherein the double-stranded polynucleotide encodes an array of three, four, five, six or more functional guide-RNA molecules

68. A method according to any one of embodiments 57 to 67, wherein the functional guide-RNA molecules are distinct functional guide-RNA molecules.

69. Use according to any one of embodiments 57 to 68, wherein the plurality of single-stranded oligonucleotide members comprises at least three, four, five, six or more members.

70. A method according to any one of embodiments 57 to 69, wherein the portions of the oligonucleotide members contain one or more gaps.

71. A method according to any one of embodiments 57 to 70, wherein the linear double-stranded polynucleotide is a vector comprising a selectable marker.

72. A method according to any one of embodiments 57 to 71, wherein the assembly results in a circular double-stranded polynucleotide construct of pre-determined sequence.

73. A method according to any one of embodiments 57 to 72, wherein the linear double-stranded polynucleotide comprises a promoter, or a part thereof, that, after assembly, is operably linked to the polynucleotide encoding the array of at least two functional guide-RNA molecules.

74. A method according to any one of embodiments 57 to 73, wherein the linear double-stranded polynucleotide comprises a terminator that, after assembly, is operably linked to the polynucleotide encoding the array of at least two functional guide-RNA molecules.

75. A method according to any one of embodiments 57 to 74, wherein the polynucleotide encoding the array of at least two functional guide-RNA molecules comprises a terminator that is operably linked to it.

76. A method according to any one of embodiments 57 to 75, wherein two reverse oligonucleotide members and one forward oligonucleotide member are used for a functional guide-RNA molecule or wherein two forward oligonucleotide members and one reverse oligonucleotide member are used for a functional guide-RNA molecule.

77. A method according to any one of embodiments 57 to 76, wherein in the cell a heterologous polynucleotide is present that integrates into the genome of the cell in the proximity of the break mediated by the functional complex of Cas12a-like enzyme or a Cas9-like enzyme and one of the at least two functional guide-RNA molecules.

78. A double-stranded polynucleotide encoding an array of at least two guide-RNA molecules obtainable or obtained by a method according to any one of embodiments 19 to 77, wherein each guide-RNA molecule comprises at least a guide sequence and an RNA processing sequence.

79. A double stranded polynucleotide according to embodiment 78, wherein the RNA processing sequence is a Cas12a Direct Repeat (DR) sequence, a Csy4 recognition sequence, a self-processing ribozyme or a tRNA.

80. A double stranded polynucleotide according to embodiment 78, wherein the RNA processing sequence is a Cas12a Direct Repeat (DR) sequence and is located on the 5′-part of each guide-RNA molecule, or wherein each guide sequence is flanked by Cas12a Direct Repeat (DR) sequences.

81. A double stranded polynucleotide according to embodiment 78, wherein the RNA processing sequence is a Csy4 recognition sequence, and wherein each guide sequence is flanked by Csy4 recognition sequences.

82. A double stranded polynucleotide according to embodiment 78, wherein the RNA processing sequence is a self-processing ribozyme and each guide sequence is flanked by ribozyme units.

83. A double stranded polynucleotide according to embodiment 78, wherein the RNA processing sequence is a tRNA and each guide sequence is flanked by tRNA's.

84. A cell obtainable by or obtained by a method according to any one of embodiments 19 to 77.

85. A method for the production of a double-stranded polynucleotide encoding an array of at least two guide-RNA molecules obtainable or obtained by a method according to any one of embodiments 19 to 77, wherein each guide-RNA molecule comprises at least a guide sequence and an RNA processing sequence, said method comprising, performing a method according to any one of embodiments 19 to 77 and subsequently isolating the double-stranded polynucleotide from the cell.

86. A method according to embodiment 85, wherein the RNA processing sequence is a Cas12a Direct Repeat (DR) sequence, a Csy4 recognition sequence, a self-processing ribozyme or a tRNA.

87. A method according to embodiment 85, wherein the RNA processing sequence is a Cas12a Direct Repeat (DR) sequence and is located on the 5′-part of each guide-RNA molecule, or wherein each guide sequence is flanked by Cas12a Direct Repeat (DR) sequences.

88. A method according to embodiment 85, wherein the RNA processing sequence is a Csy4 recognition sequence, and wherein each guide sequence is flanked by Csy4 recognition sequences.

89. A method according to embodiment 85, wherein the RNA processing sequence is a self-processing ribozyme and each guide sequence is flanked by ribozyme units.

90. A method according to embodiment 85, wherein the RNA processing sequence is a tRNA and each guide sequence is flanked by tRNA's.

91. A method according to any one of embodiments 85 to 90, wherein the double-stranded polynucleotide is amplified from the cell.

92. A method according to embodiment 91, wherein the double-stranded polynucleotide construct comprising the double stranded polynucleotide is rescued from the cell.

93. A method for the production of a compound of interest comprising, culturing a cell according to embodiment 84, said cell comprising a polynucleotide encoding a compound of interest, under conditions conducive to the production of the compound of interest, and optionally isolating and/or purifying the compound of interest.

### Definitions

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The terms “a” and “an” are used herein to refer to one or to more than one (i.e. to one or at least one) of the grammatical object of the article. By way of example, “an element” may mean one element or more than one element.

The word “about” or “approximately” when used in association with a numerical value (e.g. about 10) preferably means that the value may be the given value (of 10) more or less 1% of the value. CRISPR interference (CRISPRi) and CRISPR activation (CRISPRa) are a genetic techniques that allows for sequence-specific repression or activation of gene expression in prokaryotic and eukaryotic cells.

Herein, the term “multiplex” or “multiplexing” when used in the context of genome- and gene editing and regulation of expression is to be construed as targeting two or more loci in DNA simultaneously. Herein, the term “multiplex” or “multiplexing” when used in the context of expression is to be construed as expression of two or more guide-RNAs simultaneously.

A Cas9-like enzyme is an enzyme that has the same features as Cas9; it may be a natural variant or a synthetic variant. A preferred Cas9-like enzyme is Cas9. Functional in the sense of a Cas9-like enzyme means that it performs it functions in a cell; the function is not limited to creating a guided double-strand break, the break may e.g. be single stranded and enzyme may even only bind to the target polynucleotide without creating a break, thereby perturbing expression.

A Cas12a-like enzyme is an enzyme that has the same features as Cas12a; it may be a natural variant or a synthetic variant. A preferred Cas12a-like enzyme is Cas12a. Functional in the sense of a Cas12a-like enzyme means that it performs it functions in a cell; the function is not limited to creating a guided double-strand break, the break may e.g. be single stranded and enzyme may even only bind to the target polynucleotide without creating a break, thereby perturbing expression. A polynucleotide refers herein to a polymeric form of nucleotides of any length or a defined specific length-range or length, of either deoxyribonucleotides or ribonucleotides, or mixes or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, oligonucleotides and primers. A polynucleotide may comprise natural and non-natural nucleotides and may comprise one or more modified nucleotides, such as a methylated nucleotide and a nucleotide analogue or nucleotide equivalent wherein a nucleotide analogue or equivalent is defined as a residue having a modified base, and/or a modified backbone, and/or a non-natural internucleoside linkage, or a combination of these modifications. As desired, modifications to the nucleotide structure may be introduced before or after assembly of the polynucleotide. A polynucleotide may be further modified after polymerization, such as by conjugation with a labeling compound.

In general, codon optimization refers to a process of modifying a nucleic acid sequence for enhanced expression in a host cell of interest by replacing at least one codon (e.g. more than 1, 2, 3, 4, 5, 10, 15, 20, 25, 50, or more codons) of a native sequence with codons that are more frequently or most frequently used in the genes of that host cell while maintaining the native amino acid sequence. Various species exhibit particular bias for certain codons of a particular amino acid.

Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See e.g. Nakamura et al., 2000. Computer algorithms for codon optimizing a particular sequence for expression in a particular host cell are also available, such as Gene Forge (Aptagen; Jacobus, Pa.), are also available. Preferably, one or more codons (e.g. 1, 2, 3, 4, 5, 10, 15, 20, 25, 50, or more, or all codons) in a sequence encoding a Cas protein correspond to the most frequently used codon for a particular amino acid. Preferred methods for codon optimization are described in WO2006/077258 and WO2008/000632). WO2008/000632 addresses codon-pair optimization. Codon-pair optimization is a method wherein the nucleotide sequences encoding a polypeptide have been modified with respect to their codon-usage, in particular the codon-pairs that are used, to obtain improved expression of the nucleotide sequence encoding the polypeptide and/or improved production of the encoded polypeptide. Codon pairs are defined as a set of two subsequent triplets (codons) in a coding sequence. The amount of Cas protein in a composition disclosed herein may vary and may be optimized for optimal performance.

In an RNA molecule with a 5′-cap, a 7-methylguanylate residue is located on the 5′ terminus of the RNA (such as typically in mRNA in eukaryotes). RNA polymerase II (Pol II) transcribes mRNA in eukaryotes. Messenger RNA capping occurs generally as follows: The most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5′-5′ triphosphate-linked guanine at the transcript terminus. Finally, the 7-nitrogen of this terminal guanine is methylated by a methyl transferase. The terminology “not having a 5′-cap” herein is used to refer to RNA having, for example, a 5′-hydroxyl group instead of a 5′-cap. Such RNA can be referred to as “uncapped RNA”, for example. Uncapped RNA can better accumulate in the nucleus following transcription, since 5′-capped RNA is subject to nuclear export.

Ribozymes (ribonucleic acid enzymes) are RNA molecules that are capable of catalyzing specific biochemical reactions, including RNA splicing. A ribozyme herein refers to one or more RNA sequences that form secondary, tertiary, and/or quaternary structure(s) that can cleave RNA at a specific site. A ribozyme includes a “self-cleaving ribozyme, or self-processing ribozyme” that is capable of cleaving RNA at a c/s-site relative to the ribozyme sequence (i.e., auto-catalytic, or self-cleaving). The general nature of ribozyme nucleolytic activity is known to the person skilled in the art. The use of self-processing ribozymes in the production of guide-RNA's for RNA-guided nuclease systems such as CRISPR/Cas is inter alia described by Gao and Zhao, 2014.

A nucleotide analogue or equivalent typically comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. It is further preferred that the linkage between a residue in a backbone does not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen et al., 1991. Science 254, 1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar, 2005. Chem. Commun, 495-497). Since the backbone of a PNA molecule contains no charged phosphate groups, PNA-RNA hybrids are usually more stable than RNA-RNA or RNA-DNA hybrids, respectively (Egholm et al., 1993. Nature 365, 566-568).

A further preferred backbone comprises a morpholino nucleotide analog or equivalent, in which the ribose or deoxyribose sugar is replaced by a 6-membered morpholino ring. A most preferred nucleotide analog or equivalent comprises a phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage.

A further preferred nucleotide analogue or equivalent comprises a substitution of at least one of the non-bridging oxygens in the phosphodiester linkage. This modification slightly destabilizes base-pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

A further preferred nucleotide analogue or equivalent comprises one or more sugar moieties that are mono- or disubstituted at the 2′, 3′ and/or 5′ position such as a —OH; —F; substituted or unsubstituted, linear or branched lower (C1-C10) alkyl, alkenyl, alkynyl, alkaryl, allyl, aryl, or aralkyl, that may be interrupted by one or more heteroatoms; O-, S-, or N-alkyl; O-, S-, or N-alkenyl; O-, S- or N-alkynyl; O-, S-, or N-allyl; O-alkyl-O-alkyl, -methoxy, -aminopropoxy; aminoxy, methoxyethoxy; -dimethylaminooxyethoxy; and -dimethylaminoethoxyethoxy. The sugar moiety can be a pyranose or derivative thereof, or a deoxypyranose or derivative thereof, preferably a ribose or a derivative thereof, or deoxyribose or derivative thereof. Such preferred derivatized sugar moieties comprise Locked Nucleic Acid (LNA), in which the 2′-carbon atom is linked to the 3′ or 4′ carbon atom of the sugar ring thereby forming a bicyclic sugar moiety. A preferred LNA comprises 2′-0,4′-C-ethylene-bridged nucleic acid (Morita et al. 2001. Nucleic Acid Res Supplement No. 1: 241-242). These substitutions render the nucleotide analogue or equivalent RNase H and nuclease resistant and increase the affinity for the target.

“Sequence identity” or “identity” in the context of the disclosure of an amino acid- or nucleic acid-sequence is herein defined as a relationship between two or more amino acid (peptide, polypeptide, or protein) sequences or two or more nucleic acid (nucleotide, oligonucleotide, polynucleotide) sequences, as determined by comparing the sequences. In the art, “identity” also means the degree of sequence relatedness between amino acid or nucleotide sequences, as the case may be, as determined by the match between strings of such sequences. Herein, sequence identity with a particular sequence preferably means sequence identity over the entire length of said particular polypeptide or polynucleotide sequence.

“Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole sequence (SEQ ID NO:) as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons. Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

A polynucleotide herein is represented by a nucleotide sequence. A polypeptide herein is represented by an amino acid sequence. A nucleic acid herein is defined as a polynucleotide which is isolated from a naturally occurring gene or which has been modified to contain segments of polynucleotides which are combined or juxtaposed in a manner which would not otherwise exist in nature.

The sequence information as provided herein should not be so narrowly construed as to require inclusion of erroneously identified bases. The skilled person is capable of identifying such erroneously identified bases and knows how to correct for such errors.

A compound of interest in the context of all embodiments disclosed herein may be any biological compound. The biological compound may be biomass or a biopolymer or a metabolite. The biological compound may be encoded by a single polynucleotide or a series of polynucleotides composing a biosynthetic or metabolic pathway or may be the direct result of the product of a single polynucleotide or products of a series of polynucleotides, the polynucleotide may be a gene, the series of polynucleotide may be a gene cluster. In all embodiments disclosed herein, the single polynucleotide or series of polynucleotides encoding the biological compound of interest or the biosynthetic or metabolic pathway associated with the biological compound of interest, are preferred targets for the compositions and methods disclosed herein. The biological compound may be native to the host cell or heterologous to the host cell.

The term “heterologous biological compound” is defined herein as a biological compound which is not native to the cell; or a native biological compound in which structural modifications have been made to alter the native biological compound.

The term “biopolymer” is defined herein as a chain (or polymer) of identical, similar, or dissimilar subunits (monomers). The biopolymer may be any biopolymer. The biopolymer may for example be, but is not limited to, a nucleic acid, polyamine, polyol, polypeptide (or polyamide), or polysaccharide.

The biopolymer may be a polypeptide. The polypeptide may be any polypeptide having a biological activity of interest. The term “polypeptide” is not meant herein to refer to a specific length of the encoded product and, therefore, encompasses peptides, oligopeptides, and proteins. The term polypeptide refers to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. Polypeptides further include naturally occurring allelic and engineered variations of the above-mentioned polypeptides and hybrid polypeptides. The polypeptide may be native or may be heterologous to the host cell. The polypeptide may be a collagen or gelatine, or a variant or hybrid thereof. The polypeptide may be an antibody or parts thereof, an antigen, a clotting factor, an enzyme, a hormone or a hormone variant, a receptor or parts thereof, a regulatory protein, a structural protein, a reporter, or a transport protein, protein involved in secretion process, protein involved in folding process, chaperone, peptide amino acid transporter, glycosylation factor, transcription factor, synthetic peptide or oligopeptide, intracellular protein. The intracellular protein may be an enzyme such as, a protease, ceramidases, epoxide hydrolase, aminopeptidase, acylases, aldolase, hydroxylase, aminopeptidase, lipase. The polypeptide may also be an enzyme secreted extracellularly. Such enzymes may belong to the groups of oxidoreductase, transferase, hydrolase, lyase, isomerase, ligase, catalase, cellulase, chitinase, cutinase, deoxyribonuclease, dextranase, esterase. The enzyme may be a carbohydrase, e.g. cellulases such as endoglucanases, β-glucanases, cellobiohydrolases or β-glucosidases, hemicellulases or pectinolytic enzymes such as xylanases, xylosidases, mannanases, galactanases, galactosidases, pectin methyl esterases, pectin lyases, pectate lyases, endo polygalacturonases, exopolygalacturonases rhamnogalacturonases, arabanases, arabinofuranosidases, arabinoxylan hydrolases, galacturonases, lyases, or amylolytic enzymes; hydrolase, isomerase, or ligase, phosphatases such as phytases, esterases such as lipases, proteolytic enzymes, oxidoreductases such as oxidases, transferases, or isomerases. The enzyme may be a phytase. The enzyme may be an aminopeptidase, asparaginase, amylase, a maltogenic amylase, carbohydrase, carboxypeptidase, endo-protease, metallo-protease, serine-protease catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, protein deaminase, invertase, laccase, lipase, mannosidase, mutanase, oxidase, pectinolytic enzyme, peroxidase, phospholipase, galactolipase, chlorophyllase, polyphenoloxidase, ribonuclease, transglutaminase, or glucose oxidase, hexose oxidase, monooxygenase.

Herein, a compound of interest can be a polypeptide or enzyme with improved secretion features as described in WO2010/102982. Herein, a compound of interest can be a fused or hybrid polypeptide to which another polypeptide is fused at the N-terminus or the C-terminus of the polypeptide or fragment thereof. A fused polypeptide is produced by fusing a nucleic acid sequence (or a portion thereof) encoding one polypeptide to a nucleic acid sequence (or a portion thereof) encoding another polypeptide.

Techniques for producing fusion polypeptides are known in the art, and include, ligating the coding sequences encoding the polypeptides so that they are in frame and expression of the fused polypeptide is under control of the same promoter(s) and terminator. The hybrid polypeptides may comprise a combination of partial or complete polypeptide sequences obtained from at least two different polypeptides wherein one or more may be heterologous to the host cell. Example of fusion polypeptides and signal sequence fusions are for example as described in WO2010/121933.

The biopolymer may be a polysaccharide. The polysaccharide may be any polysaccharide, including, but not limited to, a mucopolysaccharide (e. g., heparin and hyaluronic acid) and nitrogen-containing polysaccharide (e.g., chitin). In a preferred option, the polysaccharide is hyaluronic acid.

A polynucleotide coding for the compound of interest or coding for a compound involved in the production of the compound of interest disclosed herein may encode an enzyme involved in the synthesis of a primary or secondary metabolite, such as organic acids, carotenoids, (beta-lactam) antibiotics, and vitamins. Such metabolite may be considered as a biological compound according to the disclosure.

The term “metabolite” encompasses both primary and secondary metabolites; the metabolite may be any metabolite. Preferred metabolites are citric acid, gluconic acid, adipic acid, fumaric acid, itaconic acid and succinic acid.

A metabolite may be encoded by one or more genes, such as in a biosynthetic or metabolic pathway. Primary metabolites are products of primary or general metabolism of a cell, which are concerned with energy metabolism, growth, and structure. Secondary metabolites are products of secondary metabolism (see, for example, R. B. Herbert, The Biosynthesis of Secondary Metabolites, Chapman and Hall, New York, 1981).

A primary metabolite may be, but is not limited to, an amino acid, fatty acid, nucleoside, nucleotide, sugar, triglyceride, or vitamin.

A secondary metabolite may be, but is not limited to, an alkaloid, coumarin, flavonoid, polyketide, quinine, steroid, peptide, or terpene. The secondary metabolite may be an antibiotic, antifeedant, attractant, bacteriocide, fungicide, hormone, insecticide, or rodenticide. Preferred antibiotics are cephalosporins and beta-lactams. Other preferred metabolites are exo-metabolites. Examples of exo-metabolites are Aurasperone B, Funalenone, Kotanin, Nigragillin, Orlandin, Other naphtho-γ-pyrones, Pyranonigrin A, Tensidol B, Fumonisin B2 and Ochratoxin A.

The biological compound may also be the product of a selectable marker. A selectable marker is a product of a polynucleotide of interest which product provides for biocide or viral resistance, resistance to heavy metals, prototrophy to auxotrophs, and the like. Selectable markers include, but are not limited to, amdS (acetamidase), argB (ornithinecarbamoyltransferase), bar (phosphinothricinacetyltransferase), hygB (hygromycin phosphotransferase), niaD (nitrate reductase), pyrG (orotidine-5′-phosphate decarboxylase), sC (sulfate adenyltransferase), trpC (anthranilate synthase), ble (phleomycin resistance protein), hyg (hygromycin), NAT or NTC (Nourseothricin) as well as equivalents thereof.

Herein, a compound of interest is preferably a polypeptide as described in the list of compounds of interest.

According to another disclosed embodiment, a compound of interest is preferably a metabolite. A cell according to the disclosure may already be capable of producing a compound of interest. A cell according to the disclosure may also be provided with a homologous or heterologous nucleic acid construct that encodes a polypeptide wherein the polypeptide may be the compound of interest or a polypeptide involved in the production of the compound of interest. The person skilled in the art knows how to modify a microbial host cell such that it is capable of producing a compound of interest.

All embodiments herein refer to a cell, not to a cell-free in vitro system; in other words, the systems disclosed are cell systems, not cell-free in vitro systems.

In all embodiments herein, the cell may be a haploid, diploid or polyploid cell.

A cell according disclosed herein is interchangeably herein referred as “a cell”, a “cell herein”, “a cell according to the disclosure”, “a host cell”, and as “a host cell according to the disclosure”; said cell may be any cell, a prokaryotic or a eukaryotic cell. Preferably, the cell is not a mammalian cell. Preferably the cell is a fungus, i.e. a yeast cell or a filamentous fungus cell. Preferably, the cell is deficient in an NHEJ (non-homologous end joining). The cell can be deficient in NHEJ due to the cell being deficient in a component associated with NHEJ. Said component associated with NHEJ is may be a homologue or orthologue of the yeast Ku70, Ku80, MRE11, RAD50, RAD51, RAD52, XRS2, SIR4, and/or LIG4. Alternatively, in the cell according to the disclosure, NHEJ may be rendered deficient by use of a compound that inhibits DNA ligase IV, such as SCR7 (Vartak S V and Raghavan, 2015). The person skilled in the art knows how to modulate NHEJ and its effect on RNA-guided nuclease systems, see e.g. WO2014130955A1; Chu et al., 2015; et al., 2015; Song et al., 2015 and Yu et al., 2015; all are herein incorporated by reference. The term “deficiency” is defined elsewhere herein.

When the cell according to the disclosed herein is a yeast cell, a preferred yeast cell is from a genus selected from the group consisting of Candida, Hansenula, Issatchenkia, Kluyveromyces, Pichia, Saccharomyces, Schizosaccharomyces, Yarrowia or Zygosaccharomyces; more preferably a yeast host cell is selected from the group consisting of Kluyveromyces lactis, Kluyveromyces lactis NRRL Y-1140, Kluyveromyces marxianus, Kluyveromyces. thermotolerans, Candida krusei, Candida sonorensis, Candida glabrata, Saccharomyces cerevisiae, Saccharomyces cerevisiae CEN.PK113-7D, Schizosaccharomyces pombe, Hansenula polymorpha, Issatchenkia orientalis, Yarrowia lipolytica, Yarrowia lipolytica CLIB122, Pichia stipidis and Pichia pastoris.

The host cell according to the disclosure may be a filamentous fungal host cell. Filamentous fungi as defined herein include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK).

The filamentous fungal host cell may be a cell of any filamentous form of the taxon Trichocomaceae (as defined by Houbraken and Samson in Studies in Mycology 70: 1-51. 2011). In another preferred embodiment, the filamentous fungal host cell may be a cell of any filamentous form of any of the three families Aspergillaceae, Thermoascaceae and Trichocomaceae, which are accommodated in the taxon Trichocomaceae.

The filamentous fungi are characterized by a mycelial wall composed of chitin, cellulose, glucan, chitosan, mannan, and other complex polysaccharides. Vegetative growth is by hyphal elongation and carbon catabolism is obligatory aerobic. Filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mortierella, Mucor, Myceliophthora, Neocallimastix, Neurospora, Paecilomyces, Penicillium, Piromyces, Panerochaete, Pleurotus, Schizophyllum, Talaromyces, Rasamsonia, Thermoascus, Thielavia, Tolypocladium, and Trichoderma. A preferred filamentous fungal host cell herein is from a genus selected from the group consisting of Acremonium, Aspergillus, Chrysosporium, Myceliophthora, Penicillium, Talaromyces, Rasamsonia, Thielavia, Fusarium and Trichoderma; more preferably from a species selected from the group consisting of Aspergillus niger, Acremonium alabamense, Aspergillus awamori, Aspergillus foetidus, Aspergillus sojae, Aspergillus fumigatus, Talaromyces emersonii, Rasamsonia emersonii, Rasamsonia emersonii CBS393.64, Aspergillus oryzae, Chrysosporium lucknowense, Fusarium oxysporum, Mortierella alpina, Mortierella alpina ATCC 32222, Myceliophthora thermophila, Trichoderma reesei, Thielavia terrestris, Penicillium chrysogenum and P. chrysogenum Wisconsin 54-1255 (ATCC28089); even more preferably the filamentous fungal host cell herein is an Aspergillus niger. When the host cell herein is an Aspergillus niger host cell, the host cell preferably is CBS 513.88, CBS124.903 or a derivative thereof.

Several strains of filamentous fungi are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), Agricultural Research Service Patent Culture Collection, Northern Regional Research Center (NRRL), and All-Russian Collection of Microorganisms of Russian Academy of Sciences, (abbreviation in Russian—VKM, abbreviation in English—RCM), Moscow, Russia. Preferred strains as host cells are Aspergillus niger CBS 513.88, CBS124.903, Aspergillus oryzae ATCC 20423, IFO 4177, ATCC 1011, CBS205.89, ATCC 9576, ATCC14488-14491, ATCC 11601, ATCC12892, P. chrysogenum CBS 455.95, P. chrysogenum Wisconsin54-1255 (ATCC28089), Penicillium citrinum ATCC 38065, Penicillium chrysogenum P2, Thielavia terrestris NRRL8126, Rasamsonia emersonii CBS393.64, Talaromyces emersonii CBS 124.902, Acremonium chrysogenum ATCC 36225 or ATCC 48272, Trichoderma reesei ATCC 26921 or ATCC 56765 or ATCC 26921, Aspergillus sojae ATCC11906, Myceliophthora thermophila C1, Garg 27K, VKM-F 3500 D, Chrysosporium lucknowense C1, Garg 27K, VKM-F 3500 D, ATCC44006 and derivatives thereof.

Preferably, a host cell herein has a modification, preferably in its genome which results in a reduced or no production of an undesired compound as defined herein if compared to the parent host cell that has not been modified, when analysed under the same conditions.

A modification can be introduced by any means known to the person skilled in the art, such as but not limited to classical strain improvement, random mutagenesis followed by selection. Modification can also be introduced by site-directed mutagenesis.

Modification may be accomplished by the introduction (insertion), substitution (replacement) or removal (deletion) of one or more nucleotides in a polynucleotide sequence. A full or partial deletion of a polynucleotide coding for an undesired compound such as a polypeptide may be achieved. An undesired compound may be any undesired compound listed elsewhere herein; it may also be a protein and/or enzyme in a biological pathway of the synthesis of an undesired compound such as a metabolite. Alternatively, a polynucleotide coding for said undesired compound may be partially or fully replaced with a polynucleotide sequence which does not code for said undesired compound or that codes fora partially or fully inactive form of said undesired compound. In another alternative, one or more nucleotides can be inserted into the polynucleotide encoding said undesired compound resulting in the disruption of said polynucleotide and consequent partial or full inactivation of said undesired compound encoded by the disrupted polynucleotide.

In an embodiment, the host cell herein comprises a modification in its genome selected from

a) a full or partial deletion of a polynucleotide encoding an undesired compound,
b) a full or partial replacement of a polynucleotide encoding an undesired compound with a polynucleotide sequence which does not code for said undesired compound or that codes for a partially or fully inactive form of said undesired compound.
c) a disruption of a polynucleotide encoding an undesired compound by the insertion of one or more nucleotides in the polynucleotide sequence and consequent partial or full inactivation of said undesired compound by the disrupted polynucleotide.

This modification may for example be in a coding sequence or a regulatory element required for the transcription or translation of said undesired compound. For example, nucleotides may be inserted or removed so as to result in the introduction of a stop codon, the removal of a start codon or a change or a frame-shift of the open reading frame of a coding sequence. The modification of a coding sequence or a regulatory element thereof may be accomplished by site-directed or random mutagenesis, DNA shuffling methods, DNA reassembly methods, gene synthesis (see for example Young and Dong, (2004), Nucleic Acids Research 32(7) or Gupta et al. (1968), Proc. Natl. Acad. Sci USA, 60: 1338-1344; Scarpulla et al. (1982), Anal. Biochem. 121: 356-365; Stemmer et al. (1995), Gene 164: 49-53), or PCR generated mutagenesis in accordance with methods known in the art. Examples of random mutagenesis procedures are well known in the art, such as for example chemical (NTG for example) mutagenesis or physical (UV for example) mutagenesis. Examples of site-directed mutagenesis procedures are the QuickChange™ site-directed mutagenesis kit (Stratagene Cloning Systems, La Jolla, Calif.), the ‘The Altered Sites® II in vitro Mutagenesis Systems’ (Promega Corporation) or by overlap extension using PCR as described in Gene. 1989 Apr. 15; 77(1):51-9. (Ho S N, Hunt H D, Horton R M, Pullen J K, Pease L R “Site-directed mutagenesis by overlap extension using the polymerase chain reaction”) or using PCR as described in Molecular Biology: Current Innovations and Future Trends. (Eds. A. M. Griffin and H. G. Griffin. ISBN 1-898486-01-8; 1995 Horizon Scientific Press, PO Box 1, Wymondham, Norfolk, U.K.).

Preferred methods of modification are based on recombinant genetic manipulation techniques such as partial or complete gene replacement or partial or complete gene deletion.

For example, in case of replacement of a polynucleotide, nucleic acid construct or expression cassette, an appropriate DNA sequence may be introduced at the target locus to be replaced. The appropriate DNA sequence is preferably present on a cloning vector. Preferred integrative cloning vectors comprise a DNA fragment, which is homologous to the polynucleotide and/or has homology to the polynucleotides flanking the locus to be replaced for targeting the integration of the cloning vector to this pre-determined locus. In order to promote targeted integration, the cloning vector is preferably linearized prior to transformation of the cell. Preferably, linearization is performed such that at least one but preferably either end of the cloning vector is flanked by sequences homologous to the DNA sequence (or flanking sequences) to be replaced. This process is called homologous recombination and this technique may also be used in order to achieve (partial) gene deletion.

For example a polynucleotide corresponding to the endogenous polynucleotide may be replaced by a defective polynucleotide; that is a polynucleotide that fails to produce a (fully functional) polypeptide. By homologous recombination, the defective polynucleotide replaces the endogenous polynucleotide. It may be desirable that the defective polynucleotide also encodes a marker, which may be used for selection of transformants in which the nucleic acid sequence has been modified. Alternatively or in combination with other mentioned techniques, a technique based on recombination of cosmids in an E. coli cell can be used, as described in: A rapid method for efficient gene replacement in the filamentous fungus Aspergillus nidulans (2000) Chaveroche, M-K, Ghico, J-M. and d'Enfert C; Nucleic acids Research, vol 28, no 22.

Alternatively, modification, wherein said host cell produces less of or no protein such as the polypeptide having amylase activity, preferably α-amylase activity as described herein and encoded by a polynucleotide as described herein, may be performed by established anti-sense techniques using a nucleotide sequence complementary to the nucleic acid sequence of the polynucleotide. More specifically, expression of the polynucleotide by a host cell may be reduced or eliminated by introducing a nucleotide sequence complementary to the nucleic acid sequence of the polynucleotide, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. An example of expressing an antisense-RNA is shown in Appl. Environ. Microbiol. 2000 February; 66(2):775-82. (Characterization of a foldase, protein disulfide isomerase A, in the protein secretory pathway of Aspergillus niger. Ngiam C, Jeenes D J, Punt P J, Van Den Hondel C A, Archer D B) or (Zrenner R, Willmitzer L, Sonnewald U. Analysis of the expression of potato uridinediphosphate-glucose pyrophosphorylase and its inhibition by antisense RNA. Planta. (1993); 190(2):247-52).

A modification resulting in reduced or no production of undesired compound is preferably due to a reduced production of the mRNA encoding said undesired compound if compared with a parent microbial host cell which has not been modified and when measured under the same conditions. A modification which results in a reduced amount of the mRNA transcribed from the polynucleotide encoding the undesired compound may be obtained via the RNA interference (RNAi) technique (Mouyna et al., 2004). In this method identical sense and antisense parts of the nucleotide sequence, which expression is to be affected, are cloned behind each other with a nucleotide spacer in between, and inserted into an expression vector. After such a molecule is transcribed, formation of small nucleotide fragments will lead to a targeted degradation of the mRNA, which is to be affected. The elimination of the specific mRNA can be to various extents. The RNA interference techniques described in e.g. WO2008/053019, WO2005/05672A1 and WO2005/026356A1.

A modification which results in decreased or no production of an undesired compound can be obtained by different methods, for example by an antibody directed against such undesired compound or a chemical inhibitor or a protein inhibitor or a physical inhibitor (Tour O. et al, (2003) Nat. Biotech: Genetically targeted chromophore-assisted light inactivation. Vol. 21. no. 12:1505-1508) or peptide inhibitor or an anti-sense molecule or RNAi molecule (R. S. Kamath_et al, (2003) Nature: Systematic functional analysis of the Caenorhabditis elegans genome using RNAi. Vol. 421, 231-237).

In addition of the above-mentioned techniques or as an alternative, it is also possible to inhibiting the activity of an undesired compound, or to re-localize the undesired compound such as a protein by means of alternative signal sequences (Ramon de Lucas, J., Martinez O, Perez P., Isabel Lopez, M., Valenciano, S. and Laborda, F. The Aspergillus nidulans carnitine carrier encoded by the acuH gene is exclusively located in the mitochondria. FEMS Microbiol Lett. 2001 Jul. 24; 201(2):193-8) or retention signals (Derkx, P. M. and Madrid, S. M. The foldase CYPB is a component of the secretory pathway of Aspergillus niger and contains the endoplasmic reticulum retention signal HEEL. Mol. Genet. Genomics. 2001 December; 266(4):537-545), or by targeting an undesired compound such as a polypeptide to a peroxisome which is capable of fusing with a membrane-structure of the cell involved in the secretory pathway of the cell, leading to secretion outside the cell of the polypeptide (e.g. as described in WO2006/040340).

Alternatively or in combination with above-mentioned techniques, decreased or no production of an undesired compound can also be obtained, e.g. by UV or chemical mutagenesis (Mattern, I. E., van Noort J. M., van den Berg, P., Archer, D. B., Roberts, I. N. and van den Hondel, C. A., Isolation and characterization of mutants of Aspergillus niger deficient in extracellular proteases. Mol Gen Genet. 1992 August; 234(2):332-6) or by the use of inhibitors inhibiting enzymatic activity of an undesired polypeptide as described herein (e.g. nojirimycin, which function as inhibitor for β-glucosidases (Carrel F. L. Y. and Canevascini G. Canadian Journal of Microbiology (1991) 37(6): 459-464; Reese E. T., Parrish F. W. and Ettlinger M. Carbohydrate Research (1971) 381-388)).

In an embodiment, the modification in the genome of the host cell is a modification in at least one position of a polynucleotide encoding an undesired compound.

A deficiency of a cell in the production of a compound, for example of an undesired compound such as an undesired polypeptide and/or enzyme is herein defined as a mutant microbial host cell which has been modified, preferably in its genome, to result in a phenotypic feature wherein the cell: a) produces less of the undesired compound or produces substantially none of the undesired compound and/or b) produces the undesired compound having a decreased activity or decreased specific activity or the undesired compound having no activity or no specific activity and combinations of one or more of these possibilities as compared to the parent host cell that has not been modified, when analysed under the same conditions.

Preferably, a modified host cell produces 1% less of the un-desired compound if compared with the parent host cell which has not been modified and measured under the same conditions, at least 5% less of the un-desired compound, at least 10% less of the un-desired compound, at least 20% less of the un-desired compound, at least 30% less of the un-desired compound, at least 40% less of the un-desired compound, at least 50% less of the un-desired compound, at least 60% less of the un-desired compound, at least 70% less of the un-desired compound, at least 80% less of the un-desired compound, at least 90% less of the un-desired compound, at least 91% less of the un-desired compound, at least 92% less of the un-desired compound, at least 93% less of the un-desired compound, at least 94% less of the un-desired compound, at least 95% less of the un-desired compound, at least 96% less of the un-desired compound, at least 97% less of the un-desired compound, at least 98% less of the un-desired compound, at least 99% less of the un-desired compound, at least 99.9% less of the un-desired compound, or most preferably 100% less of the un-desired compound.

A reference herein to a patent document or other matter which is given as prior art is not to be taken as an admission that that document or matter was known or that the information it contains was part of the common general knowledge as at the priority date of any of the claims.

The disclosure of each reference set forth herein is incorporated herein by reference in its entirety.

The invention is further illustrated by the following examples:

### EXAMPLES

In the following Examples, various embodiments of the invention are illustrated. From the above description and these Examples, one skilled in the art can make various changes and modifications of the disclosure to adapt it to various usages and conditions.

### Example 1: Multiplex Genome Editing Using a Single Double-Stranded Linear DNA Encoding a LbCpf1_crRNA_Array Expression Cassette with Three crRNAs

This example describes multiplex integration of three donor DNA expression cassettes encoding together a carotenoid production pathway (Verwaal et al., 2007) into three genomic loci (INT1, INT2, INT3) using a CRISPR/Cpf1 system. A CRISPR/Cpf1 system is applied in combination with a linear double stranded DNA fragment of a crRNA array expression cassette that is assembled in vivo in yeast into a linearized recipient vector.

When performing precision genome editing experiments, an easy readout of successful expression or expression levels of genes that were modified or introduced, for example based on a color change of the organisms in which such experiments are performed, is beneficial. When three genes, crtE, crtYB and crtl from Xanthophyllomyces dendrorhous are introduced and overexpressed in S. cerevisiae, the transformants will produce carotenoids which are colored compounds and consequently result in yellow, orange or red colored transformants (Verwaal et al., 2007). Coloring of the cells is a result of carotenoid production and can be achieved either by expressing crtE, crtYB and crtl from a vector, or by integration of the genes into genomic DNA, using promoters and terminators functional in S. cerevisiae to express these genes (Verwaal et al., 2007).

The crRNA array expression cassette specific for LbCpf1

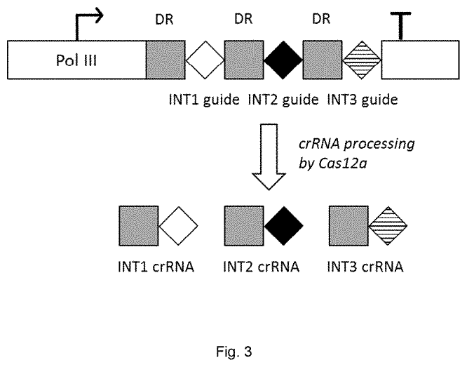

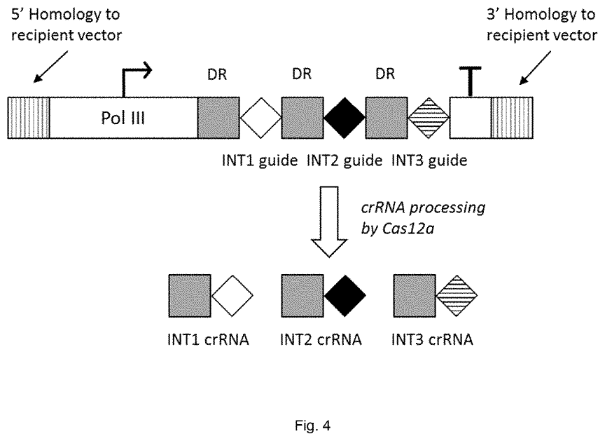

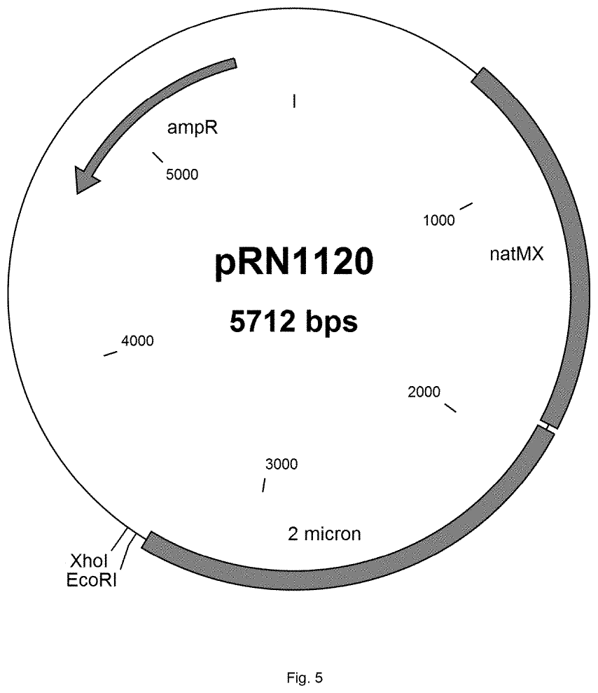

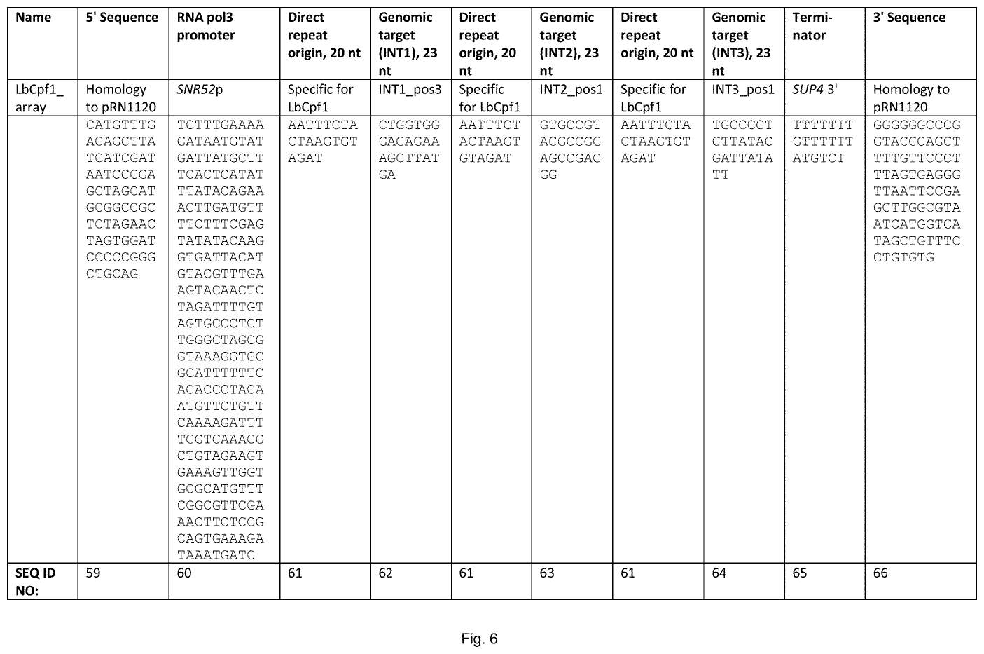

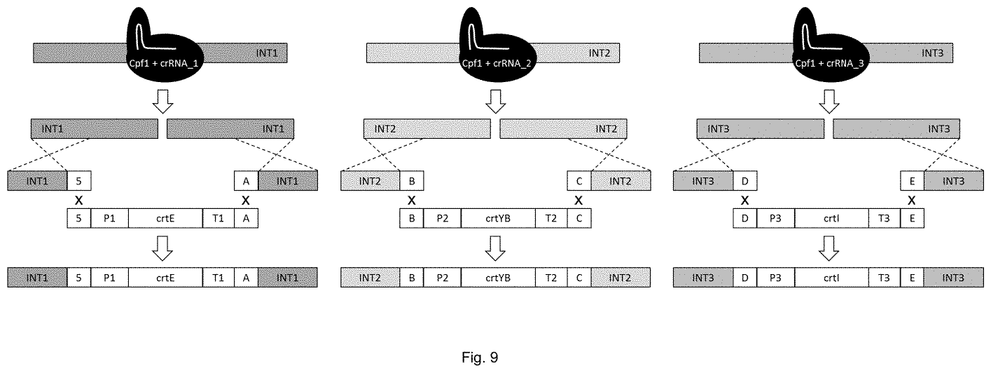

A Cpf1 crRNA array is processed by Cpf1 (Fonfara et al., 2016) to, for example, generate three individual crRNAs (as depicted in FIG. 3) to allow targeting of Cpf1 to three locations within genomic DNA (depicted in FIG. 9). A crRNA array expression cassette specific for LbCpf1 (LbCp1_crRNA_array) was designed as schematically depicted in FIG. 4. It consists of the S. cerevisiae SNR52 RNA pol III promoter (SNR52p), three units of crRNAs in their mature form each composed of a 20 bp direct repeat specific for LbCpf1 (DR_Lb) with a 23 bp guide or spacer sequence, followed by the S. cerevisiae SUP4 terminator (SUP4t). SNR52p and SUP4t sequences were derived from DiCarlo et al., (2013). The LbCp1_crRNA_array expression cassette contains homology with recipient vector pRN1120 (FIG. 5, SEQ ID NO: 1) to allow in vivo recombination of the LbCp1_crRNA_array into the linearized recipient vector. Functional crRNA sequences to target LbCpf1 to the INT1 (INT1_pos3), INT2 (INT2_pos1) or INT3 (INT3_pos1) locus were determined as described by Verwaal et al., 2018. The INT1 integration site is located at the non-coding region between NTR1 (YOR071c) and GYP1 (YOR070c) located on chromosome XV. The INT2 integration site is a non-coding region between SRP40 (YKR092C) and PTR2 (YKR093W) located on chromosome Xl. The INT3 integration site is a Ty4 long terminal repeat, located on chromosome XVI, and has been described by Flagfeldt et al. (2009). The total size of the LbCp1_crRNA_array sequence is 583 bp (SEQ ID NO: 2). The different DNA elements part of the LbCp1_crRNA_array as described above are depicted in FIG. 6 and shown in SEQ ID NO: 59-66. The LbCp1_crRNA_array sequence (SEQ ID NO: 2) was ordered at a synthetic DNA provider as a gBlock (IDT, Leuven, Belgium).

In order to obtain sufficient donor DNA for transformation to yeast, primers as set out in SEQ ID NO: 3 and SEQ ID NO: 4 were used in a PCR reaction using the LbCp1_crRNA_array expression cassette gBlock (SEQ ID NO: 2) as template. The PCR reaction was performed using Phusion as DNA polymerase (New England Biolabs, USA) in the reaction according to manufacturer's instructions. Resulting PCR products were analyzed on a 0.8% agarose gel using 1×TAE buffer (50×TAE (Tris/Acetic Acid/EDTA), 1 liter, Cat no. 1610743, BioRad, The Netherlands) and 520-Nancy (Cat no. 01494, Sigma Aldrich, Germany) to stain the PCR products. The LbCp1_crRNA_array expression cassette PCR fragment was purified using the NuceloSpin Gel and PCR Clean-up kit (Machery-Nagel, distributed by Bioké, Leiden, the Netherlands) according to manufacturer's instructions. The DNA concentration of the LbCp1_crRNA_array expression cassette PCR fragment was determined using a NanoDrop device (ThermoFisher, Life Technologies, Bleiswijk, the Netherlands), providing the concentration in nanogram per microliter.

Obtaining a S. cerevisiae Strain Expressing LbCpf1

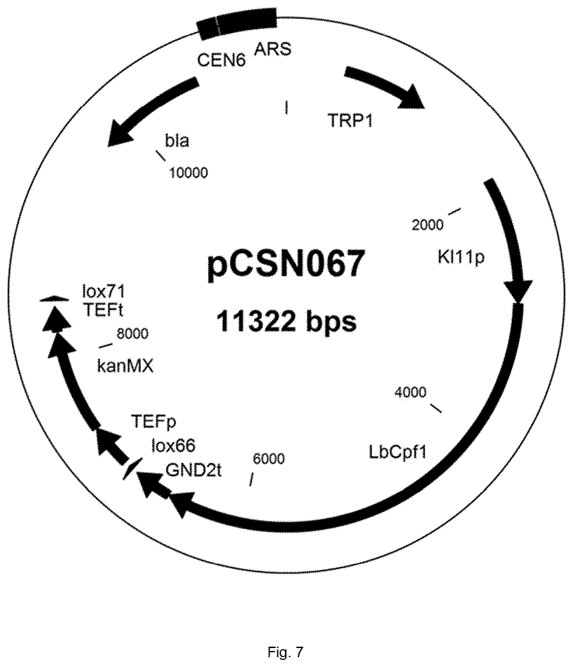

Vector pCSN067 (FIG. 7) expressing Cpf1 from Lachnospiraceae bacterium ND2006 (LbCpf1) was first transformed to S. cerevisiae strain CEN.PK113-7D (MATa URA3 HIS3 LEU2 TRP1 MAL2-SUC2) using the LiAc/salmon sperm (SS) carrier DNA/PEG method (Gietz and Woods, 2002). In the transformation mixture 1 microgram of vector pCSN067 was used. Construction of vector pCSN067 LbCpf1 is described in patent WO2017037304A2 (FIG. 21, SEQ ID NO: 88 in WO2017037304A2).

The transformation mixture was plated on YPD-agar (10 grams per liter of yeast extract, 20 grams per liter of peptone, 20 grams per liter of dextrose, 20 grams per liter of agar) containing 200 microgram (μg) G418 (Sigma Aldrich, Zwijndrecht, the Netherlands) per ml. After two to four days of growth at 30° C. colonies appeared on the transformation plate.

A yeast colony conferring resistance to G418 on the plate was inoculated on YPD-G418 medium (10 grams per liter of yeast extract, 20 grams per liter of peptone, 20 grams per liter of dextrose, 200 pg G418 (Sigma Aldrich, Zwijndrecht, the Netherlands) per ml). This transformants expressed LbCpf1.

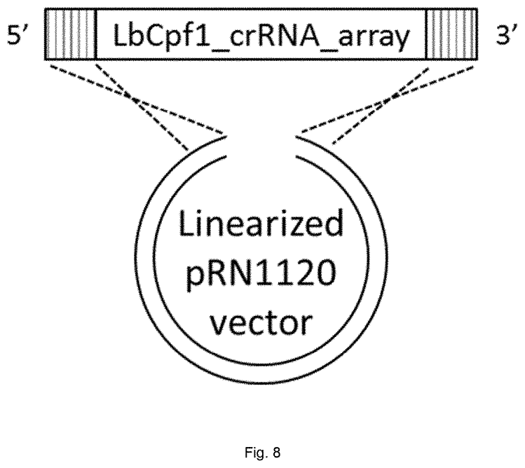

In vivo assembly of the LbCp1 crRNA array into vector pRN1120 Yeast vector pRN1120 is a multi-copy vector that contains a functional NatMX marker cassette conferring resistance against nourseothricin. The backbone of this vector is based on pRS305 (Sikorski and Hieter, 1989), including a functional 2 micron ORI sequence and a functional NatMX marker cassette (http://www.euroscarf.de). Vector pRN1120 is depicted in FIG. 5 and the sequence is set out in SEQ ID NO: 1. The LbCp1_crRNA_array expression cassette contains 78 basepairs (bp) homology at its 5′ end and 87 bp homology at its 3′ end with vector pRN1120 (after restriction of the vector with EcoRI and XhoI). The presence of homologous DNA sequences at the 5′ and 3′ end of the LbCp1_crRNA_array expression cassette will promote reconstitution of a circular vector in vivo by homologous recombination (gap repair, Orr-Weaver et al., 1983) as depicted in FIG. 8. Prior to transformation, vector pRN1120 was restricted with the restriction enzymes EcoRI and XhoI. Next, the linearized vector was purified using the NucleoSpin Gel and PCR Clean-up kit (Machery-Nagel, distributed by Bioké, Leiden, the Netherlands) according to manufacturer's instructions.

Description of Donor DNA

A donor DNA is defined herein as an extraneous polynucleotide composed of donor DNA expression cassettes or donor DNA flanks.

Donor DNA expression cassettes in this example are double-stranded DNA (dsDNA) sequences of carotenoid genes (crtE, crtYB and crtl, respectively) flanked by a functional promoter and terminator sequence. In addition, the donor DNA expression cassettes include specific 50 bp connector sequences at the 5′ and 3′ end to allow integration of the donor DNA expression cassettes into different loci within genomic DNA after recombination with the donor DNA flank sequences.

The donor DNA expression cassettes were ordered as synthetic DNA at DNA 2.0 (Menlo Park, Calif., USA) and were used as template for PCR reactions of which the products were used as donor DNA expression cassettes that were integrated into genomic DNA using the approach described in this example (Vide infra). In this example, a carotenoid gene expression cassette was composed of the following elements:
(i) at the 5′ and 3′ positions of the DNA sequence 50 bp connector sequences are present. The presence of connector sequences allowed in vivo homologous recombination between highly homologous connector sequences that are part of other donor DNA expression cassettes or donor DNA flank sequences as is described in WO2013144257A1. As a result, donor DNA fragments were assembled into the genomic DNA at a desired location and in a desired order, as is schematically depicted in FIG. 9.
(ii) A promoter sequence, which can be homologous (i.e. from S. cerevisiae) or heterologous (e.g. from Kluyveromyces lactis) and a terminator sequence derived from S. cerevisiae, were used to control the expression of the carotenogenic genes crtE, crtYB or crtl.
(iii) The crtE, crtYB and crtl nucleotide sequences were codon pair optimized for expression in S. cerevisiae as described in WO2008/000632.

Double-stranded DNA (dsDNA) donor DNA flank sequences (flanks) are used to allow integration of the carotenoid gene expression cassettes into the desired locus within the genomic DNA. The donor DNA flank sequences were composed of stretched of DNA of about 500 bp that are homologous to specific loci within genomic DNA (i.e. part of the INT1, INT2 and INT3 locus). The presence of specific 50 bp connector sequences at the 5′ or 3′ end of the donor DNA flank sequences allow integration of the donor DNA expression cassette at the desired locus, i.e. the crtE expression cassette was targeted to the INT1 locus, the crtYB sequence was targeted to the INT2 locus and the crtl sequence was targeted to the INT3 locus, as depicted in FIG. 9. Chromosomal DNA isolated from S. cerevisiae strain CEN.PK113-7D was used as template in a PCR reaction (Table 1) to obtain the dsDNA donor DNA flank sequences as PCR fragments. An overview of the different donor DNA sequences used in this experiment is provided in Table 1.

TABLE 1
Overview of different donor DNA sequences used in this experiment. Under ‘Description donor
DNA’, the following elements are indicated: Connector (Con) sequences are 50 bp DNA sequences
that are required for in vivo recombination as described in WO2013144257A1. This table includes
the SEQ ID NO's of the primers used to obtain the donor DNA sequences by PCR.

| | Description donor | Template for | Forward | Reverse |
|---|---|---|---|---|
| Donor DNA | DNA | PCR | primer | primer |
| SEQ ID NO: 5 | Expression cassette: | Synthetic DNA: | SEQ ID NO: 10 | SEQ ID NO: 11 |
| | con5 - KITDH2p - | SEQ ID NO: 5 | | |
| | crtE - ScTDH3t - | | | |
| | conA | | | |
| SEQ ID NO: 8 | Expression cassette: | Synthetic DNA: | SEQ ID NO: 12 | SEQ ID NO: 13 |
| | conB - KlYDR1p- | SEQ ID NO: 6 | | |
| | crtYB - ScPDC1t - | | | |
| | conC | | | |
| SEQ ID NO: 9 | Expression cassette: | Synthetic DNA: | SEQ ID NO: 14 | SEQ ID NO: 15 |
| | conD - ScPRE3p - | SEQ ID NO: 7 | | |
| | crtl - ScTAL1t- conE | | | |
| SEQ ID NO: 16 | Flank: INT1 5′-con5 | CEN.PK113-7D | SEQ ID NO: 22 | SEQ ID NO: 23 |
| | | genomic DNA | | |
| SEQ ID NO: 17 | Flank: conA - INT1 3′ | CEN.PK113-7D | SEQ ID NO: 24 | SEQ ID NO: 25 |
| | | genomic DNA | | |
| SEQ ID NO: 18 | Flank: INT2 5′-conB | CEN.PK113-7D | SEQ ID NO: 26 | SEQ ID NO: 27 |
| | | genomic DNA | | |
| SEQ ID NO: 19 | Flank: conC-INT2 3′ | CEN.PK113-7D | SEQ ID NO: 28 | SEQ ID NO: 29 |
| | | genomic DNA | | |
| SEQ ID NO: 20 | Flank: INT3 5′-conD | CEN.PK113-7D | SEQ ID NO: 30 | SEQ ID NO: 31 |
| | | genomic DNA | | |
| SEQ ID NO: 21 | Flank: conE-INT3 3′ | CEN.PK113-7D | SEQ ID NO: 32 | SEQ ID NO: 33 |
| | | genomic DNA | | |

PCR fragments for the donor DNA expression cassette sequences were generated using Phusion DNA polymerase (New England Biolabs, USA) according to manufacturer's instructions. In case of the expression cassettes of the carotenogenic genes, the synthetic DNA provided by DNA 2.0 (Menlo Park, Calif., USA) was used as a template in the PCR reactions, using the specific forward and reverse primer combinations depicted in Table 1. For example, in order to obtain the PCR fragment set out in SEQ ID NO: 8 (conB-crtYB-conC donor DNA expression cassette), the synthetic DNA construct provided by DNA 2.0 (SEQ ID NO: 6) was used as a template, using primer sequences set out in SEQ ID NO: 12 and SEQ ID NO: 13. In total, three different donor DNA sequences containing the carotenoid gene expression cassettes were generated by PCR, as set out in SEQ ID NO: 5, 8 and 9.

Genomic DNA (gDNA) was isolated from the yeast strain CEN.PK113-7D (MATa URA3 HIS3 LEU2 TRP1 MAL2-8 SUC2) using the lithium acetate SDS method (Lõoke et al., 2011). Strain CEN.PK113-7D is available from the EUROSCARF collection (http://www.euroscarf.de, Frankfurt, Germany) or from the Centraal Bureau voor Schimmelcultures (Utrecht, the Netherlands, entry number CBS 8340). The origin of the CEN.PK family of strains is described by van Dijken et al., 2000. This genomic DNA was used as a template to obtain the PCR fragments that were used as donor for DNA flanking sequences (comprising the overlap (complementarity, sequence identity) with the genomic DNA for genomic integration), using the specific forward and reverse primer combinations depicted in Table 1. PCR fragments for the donor DNA flank sequences were generated using Phusion DNA polymerase (New England Biolabs, USA) according to manufacturer's instructions. For example, in order to obtain the PCR fragment set out in SEQ ID NO: 16 (donor DNA flank INT1 5′-con5), genomic DNA isolated from strain CEN.PK113-7D was used as a template, using primer sequences set out in SEQ ID NO: 22 and SEQ ID NO: 23. In total, six different donor DNA sequences containing the carotenoid gene expression cassettes were generated by PCR, as set out in SEQ ID NO: 16, 17, 18, 19, 20 and 21, respectively. The donor DNA flank sequences contained 50 bp connector sequences at the 5′ or 3′ position. The presence of connector sequences allowed in vivo homologous recombination between homologous connector sequences that are part of the donor DNA expression cassettes as is described in WO2013144257A1.

Resulting PCR products were analyzed on a 0.8% agarose gel using 1×TAE buffer (50×TAE (Tris/Acetic Acid/EDTA), 1 liter, Cat no. 1610743, BioRad, The Netherlands) and 520-Nancy (Cat no. 01494, Sigma Aldrich, Germany) to stain the PCR products.

All donor DNA PCR fragments were purified using the NuceloSpin Gel and PCR Clean-up kit (Machery-Nagel, distributed by Bioké, Leiden, the Netherlands) according to manufacturer's instructions. The DNA concentration of all donor DNA fragments was determined using a NanoDrop device (ThermoFisher, Life Technologies, Bleiswijk, the Netherlands), providing the concentrations in nanogram per microliter.

Transformations

The LbCpf1 pre-expressing S. cerevisiae strain was transformed with the following DNA fragments using the LiAc/SS carrier DNA/PEG method (Gietz and Woods, 2002):
a) 100 ng of purified linearized vector pRN1120;
b) 750 ng of PCR fragment of the LbCpf1_crRNA_array expression cassette (SEQ ID NO: 2) containing homology at the 5′ and 3′ end with vector pRN1120;
c) Six donor DNA flank PCR fragments (100 ng each) with homology to the INT1 (SEQ ID NO: 16 and SEQ ID NO: 17), INT2 (SEQ ID NO: 18 and SEQ ID NO: 19) and INT3 (SEQ ID NO: 20 and SEQ ID NO: 21) integration sites;
d) Donor DNA expression cassette PCR fragments (100 ng each) encoding crtE (SEQ ID NO: 5), crtYB (SEQ ID NO: 8) and crtl (SEQ ID NO: 9).

The transformation mixtures were plated on YPD-agar (10 grams per liter of yeast extract, 20 grams per liter of peptone, 20 grams per liter of dextrose, 20 grams per liter of agar) containing 200 pg nourseothricin (NatMX, Jena Bioscience, Germany) and 200 pg G418 (Sigma Aldrich, Zwijndrecht, the Netherlands) per ml. Alternatively, transformation mixtures were plated on YPD-agar (10 grams per liter of yeast extract, 20 grams per liter of peptone, 20 grams per liter of dextrose, 20 grams per liter of agar) containing only 200 pg nourseothricin (NatMX, Jena Bioscience, Germany) per ml. After two to four days of growth at 30° C., colonies appeared on the transformation plates.

The LbCpf1_crRNA_array expression cassette, which contains 78 bp homology at the 5′-terminus and 87 bp homology at the 3′-terminus with vector pRN1120, will assemble into the linearized vector pRN1120 to form a functional circular vector (FIG. 8) by in vivo homologous recombination (gap repair, Orr-Weaver et al., 1983), which allows selection of transformants on nourseothricin. Upon expression, the LbCpf1 crRNA array is processed by Cpf1 (Fonfara et al., 2016) to generate three individual crRNAs (depicted in FIG. 3) to allow targeting of LbCpf1 to the INT1, INT2 and INT3 loci (depicted in FIG. 9).

As explained earlier in this example and in WO2013144257A1, because of the presence of homologous 50 bp connector DNA sequences, the donor DNA expression cassettes and donor DNA flank sequences will assemble to one stretch of DNA at the desired location and in the desired order into the genomic DNA to repair the double strand break introduced by Cpf1 (depicted in FIG. 9).

Transformation Results

The transformation experiment was performed twelve times. Genome editing efficiencies were determined by counting the number of colored colonies divided by the total number of transformants (colored and white colonies) on the transformation plate. Genome editing efficiencies are shown in Table 2. No colored transformants were obtained in control experiments where no LbCpf1_crRNA_array PCR fragment was included and instead of linearized vector pRN1120 a circular vector pRN1120 was used (control experiment 1). Also, no colored transformants were obtained when in addition to control experiment 1 donor DNA flank sequences were omitted from the transformation mixture. These results indicated that the carotenoid gene expression cassettes do not integrate into genomic DNA in a non-Cpf1 array-mediated fashion.

TABLE 2
Genome editing efficiencies in different transformation
experiments using a LbCpf1_crRNA_array expression
cassette obtained by PCR.

| Transformation | Genome editing |
|---|---|
| experiment | efficiency |
| 1 | 96% |
| 2 | 94% |
| 3 | 84% |
| 4 | 91% |
| 5 | 96% |
| 6 | 96% |
| 7 | 97% |
| 8 | 91% |
| 9 | 95% |
| 10 | 90% |
| 11 | 82% |
| 12 | 81% |
| Control 1 | 0% |
| Control 2 | 0% |

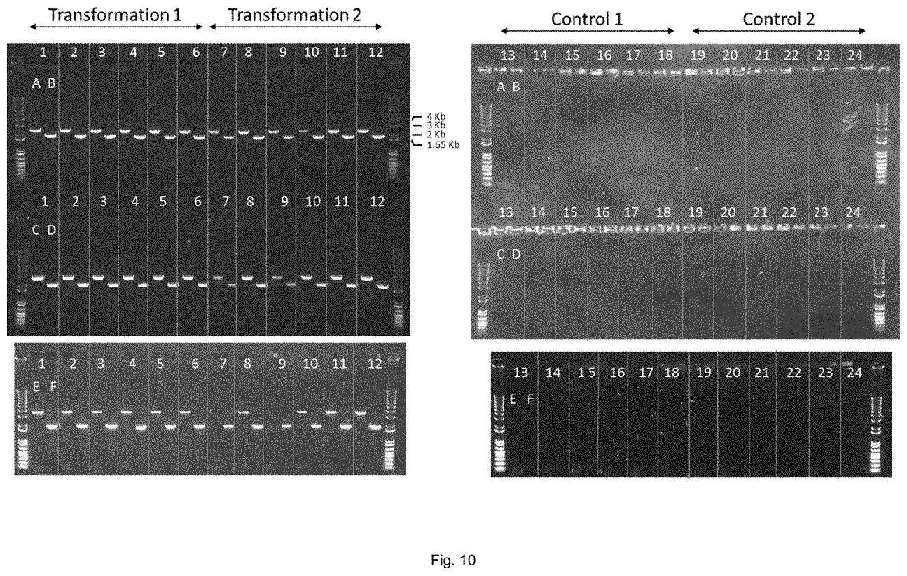

Correct integration of the donor DNA expression cassettes was verified by PCR for 6 transformants obtained from transformation experiment 1 and 6 transformants obtained from transformation experiment 2. Genomic DNA was isolated from individual transformants according to the lithium acetate SDS method (Lõoke et al., 2011). Using appropriate primers (Table 3) and Phusion DNA polymerase (New England Biolabs, USA) according to manufacturer's instructions, PCR reactions were performed. Resulting PCR products were analyzed on a 0.8% agarose gel using 1×TAE buffer (50×TAE (Tris/Acetic Acid/EDTA), 1 liter, Cat no. 1610743, BioRad, The Netherlands) and 520-Nancy (Cat no. 01494, Sigma Aldrich, Germany) to stain the PCR products. From 10 out of 12 to colored transformants, integration of the crtE, crtYB and the crtl expression cassettes into the intended loci could be confirmed (FIG. 10): a band to confirm correct integration of crtYB at the INT2 locus 5′ end (band E) was not obtained from transformation experiment 2 for transformant 7 and 9. No bands were obtained from the evaluation of 6 white transformants from control experiment 1 and 6 white colonies from control experiment 2.

TABLE 3
Primers used to confirm correct integration of carotenogenic genes into the
INT1, INT2 and INT3 loci using the LbCPf1_crRNA_array approach. To check
for the absence of genes integrated into genomic DNA, the template was replaced
by genomic DNA isolated from transformants of control experiments 1 and 2.

| | | | | Expected |
|---|---|---|---|---|
| Description | | Forward | Reverse | fragment |
| experiment | Template for PCR | primer | primer | size |
| Check correct | Genomic DNA of | SEQ ID NO: 34 | SEQ ID NO: 35 | 2295 bp |
| integration of crtE at | transformants from | | | |
| the INT1 locus 5′ | transformation | | | |
| end (band A) | experiment 1 and 2 | | | |
| Check correct | Genomic DNA of | SEQ ID NO: 36 | SEQ ID NO: 37 | 1812 bp |
| integration of crtE at | transformants from | | | |
| the INT1 locus 3′ | transformation | | | |
| end (band B) | experiment 1 and 2 | | | |
| Check correct | Genomic DNA of | SEQ ID NO: 42 | SEQ ID NO: 43 | 3406 bp |
| integration of crtYB | transformants from | | | |
| at the INT2 locus 5′ | transformation | | | |
| end (band E) | experiment 1 and 2 | | | |
| Check correct | Genomic DNA of | SEQ ID NO: 44 | SEQ ID NO: 45 | 1814 bp |
| integration of crtYB | transformants from | | | |
| at the INT2 locus 3′ | transformation | | | |
| end (band F) | experiment 1 and 2 | | | |
| Check correct | Genomic DNA of | SEQ ID NO: 38 | SEQ ID NO: 39 | 2544 bp |
| integration of crtl at | transformants from | | | |
| the INT3 locus 5′ | transformation | | | |
| end (band C) | experiment 1 and 2 | | | |
| Check correct | Genomic DNA of | SEQ ID NO: 40 | SEQ ID NO: 41 | 1817 bp |
| integration of crtl at | transformants from | | | |
| the INT3 locus 3′ | transformation | | | |
| end (band D) | experiment 1 and 2 | | | |

The experiments described in this example demonstrate that multiplex genome editing using a Cpf1 crRNA array approach, where a linear double-stranded DNA fragment encoding a crRNA array expression cassette is applied and assembles into a linearized recipient vector to allow selection for that vector in transformation, is functional in the yeast Saccharomyces cerevisiae.

### Example 2: Multiplex Genome Editing Using a LbCpf1_crRNA_Array Expression Cassette Assembled by In Vivo Oligonucleotide Assembly

This example describes multiplex integration of three donor DNA expression cassettes encoding a carotenoid production pathway (Verwaal et al., 2007) to three genomic loci (INT1, INT2, INT3).

Rather than using a double-stranded DNA (dsDNA) crRNA array including homology to recipient vector pRN1120 as a complete expression cassette, the crRNA array in this example was assembled in vivo in Saccharomyces cerevisiae using oligonucleotides and a linearized vector. In this case, the linear vector contains the SNR52 polymerase III promoter to allow expression of the crRNA array, whereas two of the oligonucleotides contained the SUP4 terminator sequence.

Construction of Recipient Vector pGRN002

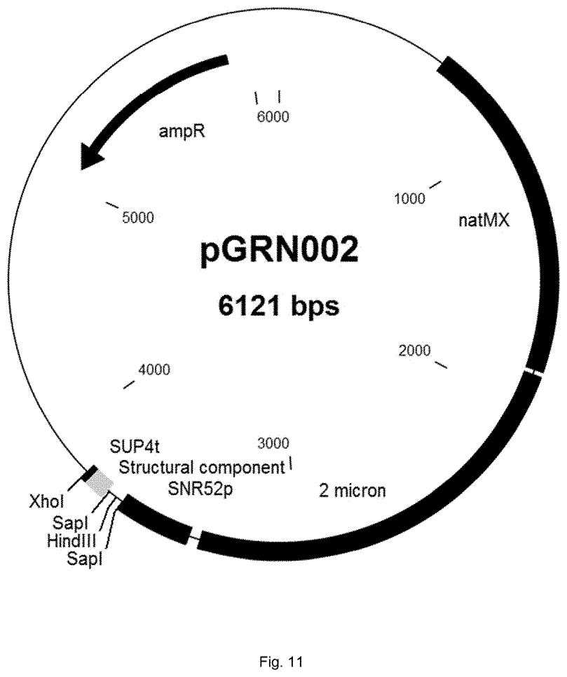

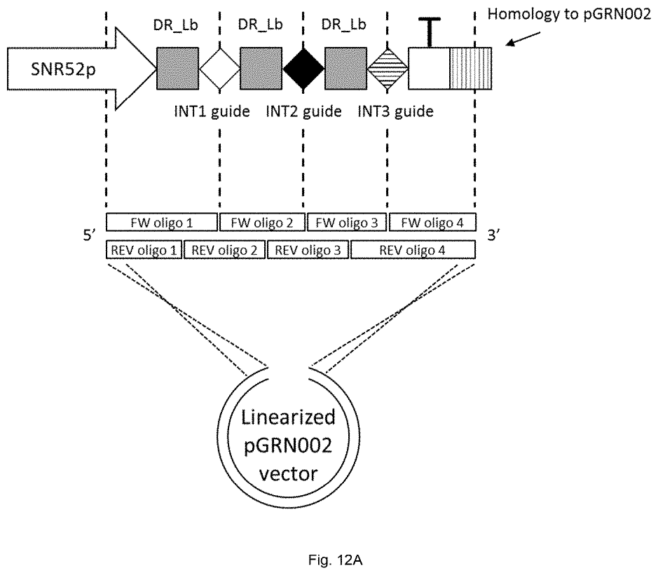

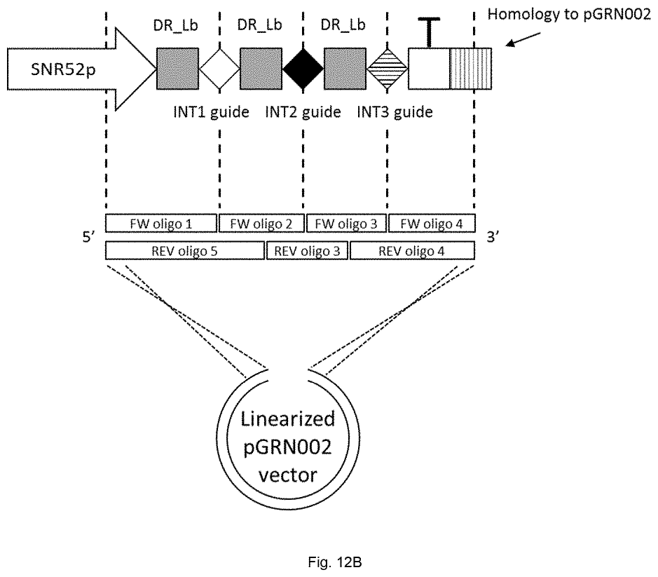

Vector pGRN002 serves as a recipient linear double-stranded DNA fragment for the in vivo oligonucleotide assembly approaches as set out in this Example. Construction of vector pGRN002 was performed as follows: The SapI restriction site was removed from vector pRN1120 (construction of PRN1120 is described in Example 1, SEQ ID NO: 1, FIG. 5) backbone by PCR using the primers set out in SEQ ID NO: 46 and SEQ ID NO: 47, changing the nucleotide sequence of the SapI restriction site from GCTCTTC to CCTCTTC. Recircularization of the intermediate PCR fragment without a SapI site was performed using the KLD enzyme mix of the Q5 site directed mutagenesis kit (New England Biolabs, supplied by Bioké, Leiden, the Netherlands. Cat no. E0554S) according to the supplier's manual. The resulting vector was digested by EcoRI and XhoI. By Gibson assembly, a gBlock containing amongst others a SNR52 promoter, a guide-RNA structural component specific for SpCas9 and a SUP4 terminator sequence (Integrated DNA Technologies, Leuven, Belgium), for which the sequence is provided in SEQ ID NO: 48, was added to the pRN1120-SapI backbone. Gibson assembly was performed using Gibson Assembly HiFi 1 Step Kit (SGI-DNA, La Jolla, Calif., USA. Cat no. GA1100-50) according to supplier's manual. The resulting vector was designated pGRN002 (SEQ ID NO: 49, FIG. 11), that amongst others contains a SNR52 polymerase III promoter, in which a crRNA array can be assembled in vivo by in a S. cerevisiae cell using oligonucleotides as explained in this Example.

In Vivo Assembly of Oligonucleotides into Vector pGRN002 to Constitute a Single crRNA Array with Three crRNAs

Prior to transformation to yeast pre-expressing Cpf1, vector pGRN0002 was restricted with SapI and XhoI, which removes the SpCas9 guide-RNA structural component and SUP4 terminator sequences (DiCarlo et al., 2013) from the vector backbone, the SNR52 RNA pol III promoter sequence (DiCarlo et al., 2013) remains present. The linearized vector was purified using the NucleoSpin Gel and PCR Clean-up kit (Machery-Nagel, distributed by Bioké, Leiden, the Netherlands) according to manufacturer's instructions. The concentration of the all DNA components was determined using a NanoDrop device (ThermoFisher, Life Technologies, Bleiswijk, the Netherlands), providing the concentration in nanogram per microliter.

The S. cerevisiae strain pre-expressing LbCpf1 from Example 1 was transformed with the following DNA components using the LiAc/SS carrier DNA/PEG method (Gietz and Woods, 2002):
a) 100 ng of purified SapI/XhoI-linearized vector pGRN002;
b) Six donor DNA flank PCR fragments (100 ng each) with homology to the INT1 (SEQ ID NO: 16 and SEQ ID NO: 17), INT2 (SEQ ID NO: 18 and SEQ ID NO: 19) and INT3 (SEQ ID NO: 20 and SEQ ID NO: 21) integration sites;
c) Donor DNA expression cassette PCR fragments (100 ng each) encoding crtE (SEQ ID NO: 5), crtYB (SEQ ID NO: 8) and crtl (SEQ ID NO: 9).
d) 500 ng of specified combinations of oligonucleotides, ordered as standard desalted primers (IDT, Leuven, Belgium), that assembled into linearized pGRN002 to constitute a crRNA array expression cassette in vivo by homologous recombination (gap repair, Orr-Weaver et al., 1983) as depicted in FIG. 12.
   Two different in vivo oligonucleotide assembly approaches were followed: Variant 1, depicted in FIG. 12A), that uses 8 single oligonucleotides; Variant 2, depicted in FIG. 12B), that uses 7 single oligonucleotides. The specific combinations of oligonucleotides in each transformation experiment is depicted in Table 4.
   The resulting crRNA expression cassette, to allow targeting of LbCpf1 to three genomic loci in S. cerevisiae genomic DNA (FIG. 9), is composed of the following DNA sequences (schematically depicted in FIG. 12): the SNR52 RNA pol III promoter, LbCpf1-specific direct repeat (DR_Lb), INT1 guide/genomic target, DR_Lb, INT2 guide, DR_Lb, INT3 guide and the SUP4 terminator. Essentially, the assembled nucleotide sequence in vector pGRN002 will be identical to the LbCpf1_crRNA_array sequence (SEQ ID NO: 2) as depicted in FIG. 6 and as applied in Example 1.

The oligonucleotides are buildup of different elements. For example, FW oligo 1 (SEQ ID NO: 50) contains homology with the SNR52 RNA pol III promoter sequence, the LbCpf1-specific direct repeat (DR_Lb) and part of the INT1 spacer/genomic target. For example, FW oligo 2 (SEQ ID NO: 51) contains part of the INT1 space/genomic target, the LbCpf1-specific direct repeat (DR_Lb) and part of the INT2 spacer/genomic target. All elements part of the oligonucleotides to constitute a crRNA array part of variant 1 are depicted in FIG. 13A) and those of variant 2 are depicted in FIG. 13B).

Upon transformation, the oligonucleotides assemble in vivo into linearized vector pGRN002 to constitute a Cpf1 crRNA array with three crRNAs, and a circular expression vector is formed that allows selection of transformants on plates containing nourseothricin. The transformation mixture was plated as described in Example 1.

TABLE 4
Oligonucleotides used for in vivo assembly into vector pGRN002
to constitute a Cpf1 crRNA array with three crRNAs.

| Variant | FW oligonucleotides | REV oligonucleotides |
|---|---|---|
| 1 | FW oligo 1 (SEQ ID NO: 50) | REV oligo 1 (SEQ ID NO: 54) |
| | FW oligo 2 (SEQ ID NO: 51) | REV oligo 2 (SEQ ID NO: 55) |
| | FW oligo 3 (SEQ ID NO: 52) | REV oligo 3 (SEQ ID NO: 56) |
| | FW oligo 4 (SEQ ID NO: 53) | REV oligo 4 (SEQ ID NO: 57) |
| 2 | FW oligo 1 (SEQ ID NO: 50) | REV oligo 3 (SEQ ID NO: 56) |
| | FW oligo 2 (SEQ ID NO: 51) | REV oligo 4 (SEQ ID NO: 57) |
| | FW oligo 3 (SEQ ID NO: 52) | REV oligo 5 (SEQ ID NO: 58) |
| | FW oligo 4 (SEQ ID NO: 53) | |

Upon expression, the LbCpf1 crRNA array is processed by Cpf1 (Fonfara et al., 2016) to generate three individual crRNAs (depicted in FIG. 3) to allow targeting of LbCpf1 to the INT1, INT2 and INT3 loci (depicted in FIG. 9).

As explained in Example 1 and in WO2013144257A1, because of the presence of homologous 50 bp connector DNA sequences, the donor DNA expression cassettes and donor DNA flank sequences will assemble to one stretch of DNA at the desired location and in the desired order into the genomic DNA to repair the double strand break introduced by Cpf1 (depicted in FIG. 9).

Transformation Results

The transformation experiment was performed six times for variant 1 and six times for variant 2. Genome editing efficiencies were determined by counting the number of colored colonies divided by the total number of transformants (colored and white colonies) on the transformation plate. Genome editing efficiencies are shown in Table 5.

TABLE 5
Genome editing efficiencies in different transformation
experiments in vivo assembly of oligonucleotides by
homologous recombination into vector pGRN002 to constitute
a Cpf1 crRNA array with three crRNAs.

| Transformation experiment | Variant | Genome editing efficiency |
|---|---|---|
| 1 | 1 | 23% |
| 2 | 1 | 12% |
| 3 | 1 | 30% |
| 4 | 1 | 12% |
| 5 | 1 | 24% |
| 6 | 1 | 14% |
| 1 | 2 | 30% |
| 2 | 2 | 21% |
| 3 | 2 | 24% |
| 4 | 2 | 22% |
| 5 | 2 | 18% |
| 6 | 2 | 15% |

The genome editing efficiency results indicate that oligonucleotides can be used to constitute a Cpf1 crRNA array expression cassette by in vivo in Saccharomyces cerevisiae to allow multiplex genome editing.

Some benefits of this approach are: 1) No PCR is required to obtain a single crRNA array expression cassette using a synthetic polynucleotide. After dissolving, the oligonucleotides ordered can be directly used in the transformation experiment. 2) Changing one of the genomic targets by another one requires changing just 3 oligonucleotides in the in vivo assembly. 3) Easy building of single crRNA arrays in a combinatorial or designed approach. 4) The number of spacer/genomic target sequences and direct repeats can be easily expanded to allow more than three multiplex genome editing events by expanding the number of oligonucleotides in the approach as described in Example 2. 5) This approach could also be used to constitute single crRNA arrays for use in other microorganisms than S. cerevisiae (e.g. Aspergillus niger or Yarrowia lipolytica). Upon assembly of the single crRNA array in S. cerevisiae, a PCR could be performed to obtain a PCR fragment of the single crRNA array expression cassette, that can be cloned into the recipient guide expression vector, or recombined in vivo into a recipient vector of the host choice.

The approach in Example 2 demonstrated the in vivo assembly of oligonucleotides into a recipient to constitute a single crRNA array expression cassette, encoding multiple guide-RNAs, which was used for multiplex genome engineering in combination with Cpf1. The approach in Example 2 can also be used for CRISPR guide-RNA expression strategies for multiplex genome engineering in combination with Cas9. By adapting the sequences of the oligonucleotides, arrays of multiple sgRNAs for example flanked by ribozymes (FIG. 1A), Csy4 cutting sites (FIG. 1B) or transfer-RNA (tRNA) sequences (FIG. 10) can be assembled in vivo into a recipient vector in S. cerevisiae.

### REFERENCES

Cress B F, Toparlak O D, Guleria S, Lebovich M, Stieglitz J T, Englaender J A, Jones J A, Linhardt R J, Koffas M A. ACS Synth Biol. 2015 Sep. 18; 4(9):987-1000. doi: 10.1021/acssynbio.5b00012. Epub 2015 Apr. 20. CRISPathBrick: Modular Combinatorial Assembly of Type II-A CRISPR Arrays for dCas9-Mediated Multiplex Transcriptional Repression in E. coli.
Deaner M, Mejia J, Alper H S. ACS Synth Biol. 2017 Oct. 20; 6(10):1931-1943. doi: 10.1021/acssynbio.7b00163. Epub 2017 Jul. 27. Enabling Graded and Large-Scale Multiplex of Desired Genes Using a Dual-Mode dCas9 Activator in Saccharomyces cerevisiae.
Deaner M, Holzman A, Alper H S. Biotechnol J. 2018 September; 13(9):e1700582. doi: 10.1002/biot.201700582. Epub 2018 Apr. 29. Modular Ligation Extension of Guide RNA Operons (LEGO) for Multiplexed dCas9 Regulation of Metabolic Pathways in Saccharomyces cerevisiae.
DiCarlo J E, Norville J E, Mali P, Rios X, Aach J, Church G M. Nucleic Acids Res. 2013 April; 41(7):4336-43. Genome engineering in Saccharomyces cerevisiae using CRISPR-Cas systems. Flagfeldt D B, Siewers V, Huang L, Nielsen J. Yeast. 2009 October; 26(10):545-51. Characterization of chromosomal integration sites for heterologous gene expression in Saccharomyces cerevisiae.
Fonfara I, Richter H, Bratovič M, Le Rhun A, Charpentier E. The CRISPR-associated DNA-cleaving enzyme Cpf1 also processes precursor CRISPR RNA. Nature. 2016 Apr. 28; 532(7600):517-21. doi: 10.1038/nature17945. Epub 2016 Apr. 20.
Gao Y, Zhao Y. J Integr Plant Biol. 2014 April; 56(4):343-9. doi: 10.1111/jipb.12152. Epub 2014 Mar. 6. Self-processing of ribozyme-flanked RNAs into guide RNAs in vitro and in vivo for CRISPR-mediated genome editing.
Gibson D G. Synthesis of DNA fragments in yeast by one-step assembly of overlapping oligonucleotides. Nucleic Acids Res. 2009 November; 37(20):6984-90. doi: 10.1093/nar/gkp687. Epub 2009 Sep. 10.
Gietz R D, Woods R A. Methods Enzymol. 2002; 350:87-96. Transformation of yeast by lithium acetate/single-stranded carrier DNA/polyethylene glycol method.
Haurwitz, R. E., Jinek, M., Wiedenheft, B., Zhou, K., and Doudna, J. A. (2010) Sequence- and structure-specific RNA processing by a CRISPR endonuclease. Science 329, 1355.
Haurwitz, R. E., Sternberg, S. H., and Doudna, J. A. (2012) Csy4 relies on an unusual catalytic dyad to position and cleave CRISPR RNA. EMBO J. 31, 2824.
Hur J K, Kim K, Been K W, Baek G, Ye S, Hur J W, Ryu S M, Lee Y S, Kim J S. Nat Biotechnol. 2016 August; 34(8):807-8. doi: 10.1038/nbt.3596. Epub 2016 Jun. 6. Targeted mutagenesis in mice by electroporation of Cpf1 ribonucleoproteins.
Kim D, Kim J, Hur J K, Been K W, Yoon S H, Kim J S. Nat Biotechnol. 2016 August; 34(8):863-8. doi: 10.1038/nbt.3609. Epub 2016 Jun. 6. Erratum in: Nat Biotechnol. 2016 Aug. 9; 34(8):888. Genome-wide analysis reveals specificities of Cpf1 endonucleases in human cells.
Kim H, Kim S T, Ryu J, Kang B C, Kim J S, Kim S G. Nat Commun. 2017 Feb. 16; 8:14406. doi: 10.1038/ncomms14406. CRISPR/Cpf1-mediated DNA-free plant genome editing.
Kim H K, Song M, Lee J, Menon A V, Jung S, Kang Y M, Choi J W, Woo E, Koh H C, Nam J W, Kim H. Nat Methods. 2017 February; 14(2):153-159. doi: 10.1038/nmeth.4104. Epub 2016 Dec. 19. In vivo high-throughput profiling of CRISPR-Cpf1 activity.
Kim Y, Cheong S A, Lee J G, Lee S W, Lee M S, Baek I J, Sung Y H. Nat Biotechnol. 2016 August; 34(8):808-10. doi: 10.1038/nbt.3614. Epub 2016 Jun. 6. Generation of knockout mice by Cpf1-mediated gene targeting.
Knott G J, Doudna J A. Science. 2018 Aug. 31; 361(6405):866-869. doi: 10.1126/science.aat5011. Review. CRISPR-Cas guides the future of genetic engineering.
Lian J, HamediRad M, Hu S, Zhao H. Nat Commun. 2017 Nov. 22; 8(1):1688. doi: 10.1038/s41467-017-01695-x. Combinatorial metabolic engineering using an orthogonal tri-functional CRISPR system.
Lian J, HamediRad M, Zhao H. Biotechnol J. 2018 September; 13(9):e1700601. doi: 10.1002/biot.201700601. Epub 2018 Apr. 18. Advancing Metabolic Engineering of Saccharomyces cerevisiae Using the CRISPR/Cas System.
Chunyu Liao, Fani Ttofali, Rebecca A Slotkowski, Steven R Denny, Taylor D Cecil, Ryan T Leenay, Albert J Keung, Chase L Beisel. doi: https://doi.org/10.1101/312421. Posted on BioRxiv on May 2, 2018. One-step assembly of large CRISPR arrays enables multi-functional targeting and reveals constraints on array design.
Lõoke M, Kristjuhan K, Kristjuhan A. Biotechniques. 2011 May; 50(5):325-8. Extraction of genomic DNA from yeasts for PCR-based applications.
Mahfouz M M. Genome editing: Nat Plants. 2017 Mar. 3; 3:17028. doi: 10.1038/nplants.2017.28. The efficient tool CRISPR-Cpf1.
Makarova K S, Wolf Y I, Alkhnbashi O S, Costa F, Shah S A, Saunders S J, Barrangou R, Brouns S J, Charpentier E, Haft D H, Horvath P, Moineau S, Mojica F J, Terns R M, Terns M P, White M F, Yakunin A F, Garrett R A, van der Oost J, Backofen R, Koonin E V. Nat Rev Microbiol. 2015 November; 13(11):722-36. doi: 10.1038/nrmicro3569. Epub 2015 Sep. 28. Review. An updated evolutionary classification of CRISPR-Cas systems.
Makarova K S, Zhang F, Koonin E V. Cell. 2017 Jan. 12; 168(1-2):328-328.e1. doi: 10.1016/j.cell.2016.12.038. Epub 2017 Jan. 12. SnapShot: Class 2 CRISPR-Cas Systems.
Nakamura Y, Gojobori T, Ikemura T. Nucleic Acids Res. 2000 Jan. 1; 28(1):292. Codon usage tabulated from international DNA sequence databases: status for the year 2000.
Nissim L, Perli S D, Fridkin A, Perez-Pinera P, Lu T K. Biotechnol J. Mol Cell. 2014 May 22; 54(4):698-710. doi: 10.1016/j.molcel.2014.04.022. Epub 2014 May 15. Multiplexed and programmable regulation of gene networks with an integrated RNA and CRISPR/Cas toolkit in human cells.
Orr-Weaver T L, Szostak J W, Rothstein R J. Methods Enzymol. 1983; 101:228-45. Genetic applications of yeast transformation with linear and gapped plasmids.
Port F, Bullock S L. Nat Methods. 2016 October; 13(10):852-4. doi: 10.1038/nmeth.3972. Epub 2016 Sep. 5. Augmenting CRISPR applications in Drosophila with tRNA-flanked sgRNAs.
Sikorski R S, Hieter P. Genetics. 1989 May; 122(1):19-27. A system of shuttle vectors and yeast host strains designed for efficient manipulation of DNA in Saccharomyces cerevisiae.
Swarts D C, Jinek M. Wiley Interdiscip Rev RNA. 2018 May 22:e1481. doi: 10.1002/wrna.1481. Review. Cas9 versus Cas12a/Cpf1: Structure-function comparisons and implications for genome editing.
Swiat M A, Dashko S, den Ridder M, Wijsman M, van der Oost J, Daran J M, Daran-Lapujade P. Nucleic Acids Res. 2017 Dec. 1; 45(21):12585-12598. doi: 10.1093/nar/gkx1007. FnCpf1: a novel and efficient genome editing tool for Saccharomyces cerevisiae.
Tak Y E, Kleinstiver B P, Nunez J K, Hsu J Y, Horng J E, Gong J, Weissman J S, Joung J K. Nat Methods. 2017 December; 14(12):1163-1166. doi: 10.1038/nmeth.4483. Epub 2017 Oct. 30. Inducible and multiplex gene regulation using CRISPR-Cpf1-based transcription factors.
van Dijken J P, Bauer J, Brambilla L, Duboc P, Francois J M, Gancedo C, Giuseppin M L, Heijnen J J, Hoare M, Lange H C, Madden E A, Niederberger P, Nielsen J, Parrou J L, Petit T, Porro D, Reuss M, van Riel N, Rizzi M, Steensma H Y, Verrips C T, Vindelov J, Pronk J T. An interlaboratory comparison of physiological and genetic properties of four Saccharomyces cerevisiae strains. Enzyme Microb Technol. 2000 Jun. 1; 26(9-10):706-714.
Vercoe R B, Chang J T, Dy R L, Taylor C, Gristwood T, Clulow J S, Richter C, Przybilski R, Pitman A R, Fineran P C. PLoS Genet. 2013 April; 9(4):e1003454. doi: 10.1371/journal.pgen.1003454. Epub 2013 Apr. 18. Cytotoxic chromosomal targeting by CRISPR/Cas systems can reshape bacterial genomes and expel or remodel pathogenicity islands.
Verwaal R, Wang J, Meijnen J P, Visser H, Sandmann G, van den Berg J A, van Ooyen A J. Appl Environ Microbiol. 2007 July; 73(13):4342-50. Epub 2007 May 11. High-level production of beta-carotene in Saccharomyces cerevisiae by successive transformation with carotenogenic genes from Xanthophyllomyces dendrorhous.
Verwaal R, Buiting-Wiessenhaan N, Dalhuijsen S, Roubos J A. Yeast. 2018 February; 35(2):201-211. doi: 10.1002/yea.3278. Epub 2017 Nov. 12. CRISPR/Cpf1 enables fast and simple genome editing of Saccharomyces cerevisiae.
Wang M, Mao Y, Lu Y, Tao X, Zhu J K. Mol Plant. 2017 Jul. 5; 10(7):1011-1013. doi: 10.1016/j.molp.2017.03.001. Epub 2017 Mar. 16. Multiplex Gene Editing in Rice Using the CRISPR-Cpf1 System.
Xu R, Qin R, Li H, Li D, Li L, Wei P, Yang J. Plant Biotechnol J. 2017 June; 15(6):713-717. doi: 10.1111/pbi.12669. Epub 2017 Feb. 19. Generation of targeted mutant rice using a CRISPR-Cpf1 system.
Zetsche B, Gootenberg J S, Abudayyeh 00, Slaymaker I M, Makarova K S, Essletzbichler P, Volz S E, Joung J, van der Oost J, Regev A, Koonin E V, Zhang F. Cell. 2015 Oct. 22; 163(3):759-71. Cpf1 is a single RNA-guided endonuclease of a class 2 CRISPR-Cas system.
Zetsche B, Heidenreich M, Mohanraju P, Fedorova I, Kneppers J, DeGennaro E M, Winblad N, Choudhury S R, Abudayyeh 00, Gootenberg J S, Wu W Y, Scott D A, Severinov K, van der Oost J, Zhang F. Nat Biotechnol. 2017 January; 35(1):31-34. doi: 10.1038/nbt.3737. Epub 2016 Dec. 5. Erratum in: Nat Biotechnol. 2017 Feb. 8; 35(2):178. Multiplex gene editing by CRISPR-Cpf1 using a single crRNA array.
Ziehler W A, Day J J, Fierke C A, Engelke D R. Biochemistry. 2000 Aug. 15; 39(32):9909-16. Effects of 5′ leader and 3′ trailer structures on pre-tRNA processing by nuclear RNase P.

1. A method for expression within a cell of at least two functional guide-RNA molecules, comprising contacting a cell with a plurality of single-stranded oligonucleotide members and a linear double-stranded polynucleotide member such that these are introduced into the cell, wherein the members of the plurality of single-stranded oligonucleotides are capable of assembly within a cell into a double-stranded polynucleotide encoding an array of at least two functional guide-RNA molecules, wherein each guide-RNA molecule comprises at least a guide sequence and an RNA processing sequence.

2. The method according to claim 1, wherein the RNA processing sequence is a Cas12a Direct Repeat (DR) sequence, a Csy4 recognition sequence, a self-processing ribozyme or a tRNA.

3. The method according to claim 1, wherein the cell expresses a functional Cas12a-like enzyme, a functional Csy4 and/or a functional Cas9-like enzyme or wherein in the cell a functional Cas12a-like enzyme, a functional Csy4 and/or a functional Cas9-like enzyme is present.

4. The method according to claim 1, wherein a part at a 5′-end of the double-stranded polynucleotide encoding at least two functional guide-RNA molecules has sequence identity with a part at one terminal part of the linear double-stranded polynucleotide and wherein a part at a 3′-end of the double-stranded polynucleotide encoding the array of at least two functional guide-RNA molecules has sequence identity with another terminal part of the linear double-stranded polynucleotide, such that a plurality of single-stranded oligonucleotide members, when assembled, can assemble together with the linear double-stranded polynucleotide into the double-stranded polynucleotide construct.

5. The method according to claim 1, wherein the oligonucleotide members comprise overlapping portions at least 10 bases each, such that they are capable of assembly within a cell into a double-stranded polynucleotide encoding an array of at least two functional guide-RNA molecules.

6. The method according to claim 1, wherein the double-stranded polynucleotide encodes an array of three, four, five, six or more functional guide-RNA molecules.

7. The method according to claim 1, wherein the plurality of single-stranded oligonucleotide members comprises at least three, four, five, six or more members.

8. The method according to claim 1, wherein the linear double-stranded polynucleotide is a vector comprising a selectable marker.

9. The method according to claim 1, wherein the assembly results in a circular double-stranded polynucleotide construct of pre-determined sequence.

10. The method according to claim 1, wherein the linear double-stranded polynucleotide comprises a promoter, or a part thereof, that, after assembly, is operably linked to the polynucleotide encoding the array of at least two functional guide-RNA molecules.

11. The method according to claim 1, wherein the linear double-stranded polynucleotide comprises a terminator that, after assembly, is operably linked to the polynucleotide encoding the array of at least two functional guide-RNA molecules.

12. The method according to claim 1, wherein the polynucleotide encoding the array of at least two functional guide-RNA molecules comprises a terminator that is operably linked thereto.

13. The method according to claim 1, wherein two reverse oligonucleotide members and one forward oligonucleotide member are used for a functional guide-RNA molecule or wherein two forward oligonucleotide members and one reverse oligonucleotide member are used for a functional guide-RNA molecule.

14. A cell obtainable by or obtained by the method according to claim 1.

15. A method for production of a compound of interest comprising, culturing a cell according to claim 14, said cell comprising a polynucleotide encoding a compound of interest, under conditions conducive to production of the compound of interest, and optionally isolating and/or purifying the compound of interest.


SEQUENCE LISTING (79 sequences)

SEQ ID NO: 1 - DNA, 5712 bp, Artificial Sequence
  Vector pRN1120
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacacctaggatccgtcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactaggggcagggcatgctcatgtagagcgcctgctcgccgtccgaggcggtgccgtcgtacagggcggtgtccaggccgcagagggtgaaccccatccgccggtacgcgtggatcgccggtgcgttgacgttggtgacctccagccagaggtgcccggcgccccgctcgcgggcgaactccgtcgcgagccccatcaacgcgcgcccgaccccgtgcccccggtgctccggggcgacctcgatgtcctcgacggtcagccggcggttccagccggagtacgagacgaccacgaagcccgccaggtcgccgtcgtccccgtacgcgacgaacgtccgggagtccgggtcgccgtcctccccggcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcattttacatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggccttaagttcgaacgtacgagctccggcattgcgaataccgctttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactaggtatagtctatagtccgtggaattaattctcatgtttgacagcttatcatcgataatccggagctagcatgcggccgctctagaactagtggatcccccgggctgcaggaattcgatatcaagcttatcgataccgtcgacctcgagggggggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 583 bp, Artificial Sequence
  Nucleotide sequence of the LbCpf1_crRNA_array nucleotide sequence including homology with plasmid pRN1120
catgtttgacagcttatcatcgataatccggagctagcatgcggccgctctagaactagtggatcccccgggctgcagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcaatttctactaagtgtagatctggtgggagagaaagcttatgaaatttctactaagtgtagatgtgccgtacgccggagccgacggaatttctactaagtgtagattgcccctcttatacgattatatttttttttgttttttatgtctggggggcccggtacccagcttttgttccctttagtgagggttaattccgagcttggcgtaatcatggtcatagctgtttcctgtgtg

SEQ ID NO: 3 - DNA, 21 bp, Artificial Sequence
  Nucleotide sequence of the FW primer to amplify a LbCpf1 crRNA array expression cassette for in vivo assembly into linearized pRN1120
catgtttgacagcttatcatc

SEQ ID NO: 4 - DNA, 21 bp, Artificial Sequence
  Nucleotide sequence of the REV primer to amplify a LbCpf1 crRNA array expression cassette for in vivo assembly into linearized pRN1120
cacacaggaaacagctatgac

SEQ ID NO: 5 - DNA, 2540 bp, Artificial Sequence
  Nucleotide sequence of the synthetic and donor DNA crtE expression cassette (con5-KlTDH2p-crtE-ScTDH3t-conA)
aagcgacttccaatcgctttgcatatccagtaccacacccacaggcgtttgtgccgtaaaaactaaaacgagcccccaccaaagaacaaaaaagaaggtgctgggcccccactttcttcccttgcacgtgataggaagatggctacagaaacaagaagatggaaatcgaaggaaagagggagactggaagctgtaaaaactgaaatgaaaaaaaaaaaaaaaaaaaaaaacaagaagctgaaaatggaagactgaaatttgaaaaatggtaaaaaaaaaaaagaaacacgaagctaaaaacctggattccattttgagaagaagcaagaaaggtaagtatggtaacgaccgtacaggcaagcgcgaaggcaaatggaaaagctggagtccggaagataatcatttcatcttcttttgttagaacagaacagtggatgtccctcatctcggtaacgtattgtccatgccctagaactctctgtccctaaaaagaggacaaaaacccaatggtttccccagcttccagtggagccaccgatcccactggaaaccactggacaggaagagaaaatcacggacttcctctattgaaggataattcaacactttcaccagatcccaaatgtcccgcccctattcccgtgttccatcacgtaccataacttaccatttcatcacgttctctatggcacactggtactgcttcgactgctttgcttcatcttctctatgggccaatgagctaatgagcacaatgtgctgcgaaataaagggatatctaatttatattattacattataatatgtactagtgtggttattggtaattgtacttaattttgatatataaagggtggatctttttcattttgaatcagaattggaattgcaacttgtctcttgtcactattacttaatagtaattatatttcttattaaccttttttttaagtcaaaacaccaaggacaagaactactcttcaaaggtatttcaagttatcatacgtgtcacacacgcttcacagtttcaagtaaaaaaaaagaatattacacaatggactacgctaacatcttgactgccattcctttggaattcaccccacaagatgacattgtcttgttggaaccataccactacttaggtaagaacccaggtaaggaaatcagatctcaattgattgaagctttcaactactggttagatgtcaagaaggaagacttggaagttatccaaaatgttgttggtatgttgcacaccgcttctttgttgatggatgatgttgaagattcttccgtcttgagaagaggttctccagttgctcatttgatctacggtattccacaaaccatcaacactgctaactacgtttacttcttggcttaccaagaaatcttcaaattgcgtccaactccaattccaatgccagttatcccaccatcttctgcttctttgcaatcttctgtctcctccgcctcctcttcctcttctgcctcctctgaaaacggtggtacctccactccaaactcccaaatcccattctccaaggacacctacttggacaaggttatcactgacgaaatgttgtctttgcaccgtggtcaaggtttggaattattctggagagactctttgacctgtccatctgaagaagaatacgtcaagatggtcttgggtaagaccggtggtttgttcagaattgctgtcagattgatgatggccaagtctgaatgtgacattgactttgttcaattggttaacttgatttccatctacttccaaatcagagatgactacatgaacttgcaatcctctgaatacgctcacaacaagaacttcgctgaagacttgactgaaggtaagttctccttcccaaccattcactccattcacgctaacccatcttccagattggttatcaacactttacaaaagaagtccacttctccagaaatcttacatcactgtgtcaactacatgagaactgaaacccactctttcgaatacactcaagaagtcttgaacactttatctggtgctttggaaagagaattgggtagattacaaggtgaatttgctgaagctaactccaagatcgatttgggtgacgttgaatctgaaggtagaaccggtaagaacgtcaaattggaagccatcttgaagaaattggctgatatccctctataaagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggatcctcttgcccatcgaacgtacaagtactcctctgttctctccttcctttgcttt

SEQ ID NO: 6 - DNA, 3431 bp, Artificial Sequence
  Nucleotide sequence of the synthetic crtYB expression cassette (conA-KlYDR2p-crtYB-ScPDC1t-conB)
ttgcccatcgaacgtacaagtactcctctgttctctccttcctttgctttgtgcttttctttttttgcggtcacccccatgtggcggggaggcagaggagtaggtagagcaacgaatcctactatttatccaaattagtctaggaactctttttctagattttttagatttgagggcaagcgctgttaacgactcagaaatgtaagcactacggagtagaacgagaaatccgccataggtggaaatcctagcaaaatcttgcttaccctagctagcctcaggtaagctagccttagcctgtcaaatttttttcaaaatttggtaagtttctactagcaaagcaaacacggttcaacaaaccgaaaactccactcattatacgtggaaaccgaaacaaaaaaacaaaaaccaaaatactcgccaatgagaaagttgctgcgtttctactttcgaggaagaggaactgagaggattgactacgaaaggggcaaaaacgagtcgtattctcccattattgtctgctaccacgcggtctagtagaataagcaaccagtcaacgctaagacaggtaatcaaaataccagtctgctggctacgggctagtttttacctcttttagaacccactgtaaaagtccgttgtaaagcccgttctcactgttggcgtttttttttttttggtttagtttcttatttttcatttttttctttcatgaccaaaaacaaacaaatctcgcgatttgtactgcggccactggggcgtggccaaaaaaatgacaaatttagaaaccttagtttctgatttttcctgttatgaggagatatgataaaaaatattactgctttattgttttttttttatctactgaaatagagaaacttacccaaggaggaggcaaaaaaaagagtatatatacagcaggtaccattcagattttaatatattcttttctcttcttctacactattattataataattttactatattcatttttagcttaaaacctcatagaatattattcttcagtcactcgcttaaatacttatcaaaaatgaccgctttggcttactaccaaatccacttgatctacactttgccaatcttaggtttgctaggtttgttgacttctccaattttgaccaaattcgacatctacaagatttctatcttagtctttattgctttctctgctaccactccatgggactcctggatcatcagaaacggtgcctggacctacccatctgctgaatctggtcaaggtgttttcggtacctttttggatgtcccatacgaagaatacgccttctttgttatccaaaccgtcatcaccggtttggtttacgttttggctaccagacatttgttgccatctttggctctaccaaagacccgttcttctgccttgtctctagctttgaaggctttaatcccattgccaatcatctatttgttcaccgctcatccatctccatccccagatcctttggttactgaccactacttctacatgagagctttgtctttgttgatcaccccaccaaccatgttgttggctgctttatctggtgaatacgctttcgactggaaatctggtagagctaagtccaccattgctgccatcatgatcccaactgtctacttgatctgggttgactacgttgccgttggtcaagactcctggtccatcaacgatgaaaagattgtcggttggagattaggtggtgtcttgccaattgaagaagctatgttcttcttattgaccaacttgatgatcgttttgggtttgtctgcctgtgaccacactcaagccttgtacttgttgcacggtagaactatctacggtaacaagaagatgccatcttctttcccattaatcactccaccagttttgtccttgttcttctcctccagaccatactcctcccaaccaaagagagatttggaattggctgtcaagttgttggaaaagaagtccagatctttcttcgttgcttctgccggtttcccatctgaagtcagagaaagattggttggtttgtacgctttctgtcgtgtcaccgatgacttgattgactctccagaagtttcctccaacccacacgctaccattgacatggtttccgatttcttgactttattattcggtcctccattgcacccatctcaaccagacaagattttgtcttctccattattaccaccttcccacccatccagaccaactggtatgtacccattaccaccacctccatctttgtctccagctgaattggtccaattcttgactgaacgtgtcccagttcaataccacttcgctttcagattgttggccaaattgcaaggtttgattccaagatacccattggatgaattattgagaggttacaccactgacttgatcttcccattgtccactgaagccgtccaagctagaaagaccccaattgaaactactgctgacttgttggactacggtttgtgtgttgccggttctgttgctgaattgttggtctacgtttcctgggcttccgctccatcccaagttccagctactattgaagaaagagaagctgttttggtcgcctctcgtgaaatgggtaccgctttgcaattggtcaacattgccagagatatcaagggtgacgctactgaaggtagattctacttgccattgtctttctttggtttgagagatgaatccaaattggccattccaactgactggactgaaccaagacctcaagatttcgacaaattgttgtctctatctccatcttccactttaccatcctctaacgcttctgaatccttcagattcgaatggaagacctactctttgccattggttgcttacgctgaagatttggctaagcactcttacaagggtattgacagattaccaactgaagtccaagctggtatgagagctgcttgtgcttcttacttgttgattggtcgtgaaatcaaggttgtctggaagggtgatgtcggtgaaagaagaaccgttgctggttggagaagagtcagaaaggttttgtctgttgtcatgtccggttgggaaggtcaataaagcgatttaatctctaattattagttaaagttttataagcatttttatgtaacgaaaaataaattggttcatattattactgcactgtcacttaccatggaaagaccagacaagaagttgccgacagtctgttgaattggcctggttaggcttaagtctgggtccgcttctttacaaatttggagaatttctcttaaacgatatgtatattcttttcgttggaaaagatgtcttccaaaaaaaaaaccgatgaattagtggaaccaaggaaaaaaaaagaggtatccttgattaaggaacacctccggatcgatgtacacaaccgactgcacccaaacgaacacaaatcttagca

SEQ ID NO: 7 - DNA, 2758 bp, Artificial Sequence
  Nucleotide sequence of the synthetic crtI expression cassette (conB-ScPRE3p-crtI-ScTAL1t-conC)
cggatcgatgtacacaaccgactgcacccaaacgaacacaaatcttagcagtgccaaacattaatttgttctgcatactttgaacctttcagaaaataaaaaacattacgcgcatacttaccctgctcgcgaagaagagtaacactaacgcattctatgggcaattgaagacagtattcagtacaagacatagtccgtttccttgagtcaattcctatagcattatgaactagccgcctttaagagtgccaagctgttcaacaccgatcatttttgatgatttggcgtttttgttatattgatagatttcttttgaattttgtcattttcacttttccactcgcaacggaatccggtggcaaaaaagggaaaagcattgaaatgcaatctttaacagtattttaaacaagttgcgacacggtgtacaattacgataagaattgctacttcaaagtacacacagaaagttaacatgaatggaattcaagtggacatcaatcgtttgaaaaagggcgaagtcagtttaggtacctcaatgtatgtatataagaatttttcctcccactttattgtttctaaaagttcaatgaagtaaagtctcaattggccttattactaactaataggtatcttataatcacctaataaaatagaatgggtaaggaacaagaccaagacaagccaactgccatcatcgttggttgtggtatcggtggtattgctaccgctgccagattagctaaggaaggtttccaagttaccgtctttgaaaagaacgactactccggtggtagatgttctttgattgaaagagatggttacagattcgaccaaggtccatctttgttgctattaccagacttgttcaagcaaaccttcgaagatttgggtgaaaagatggaagactgggttgatttgatcaagtgtgaaccaaactacgtttgtcacttccatgatgaagaaactttcaccttctccactgacatggctttattgaagagagaagtcgaaagatttgaaggtaaagatggtttcgacagattcttgtctttcatccaagaagctcacagacattacgaattggctgttgtccacgtcttgcaaaagaacttcccaggtttcgctgctttcttgagattacaattcatcggtcaaatcttagctttgcacccatttgaatccatctggaccagagtttgtcgttacttcaagactgacagattgagaagagtcttctcctttgccgttatgtacatgggtcaatctccatactctgctccaggtacctactccttgttgcaatacactgaattgactgaaggtatctggtacccaagaggtggtttctggcaagttccaaacactttgttgcaaatcgtcaagagaaacaacccatctgctaagttcaacttcaacgctccagtttctcaagttttgttgtctccagctaaggacagagctaccggtgtcagattagaatctggtgaagaacaccacgctgatgttgtcattgtcaatgctgacttggtctacgcttctgaacatttgattccagatgatgctagaaacaagatcggtcaattaggtgaagttaagcgttcctggtgggctgatttggttggtggtaagaagttgaagggttcttgttcttctttgtctttctactggtctatggacagaatcgttgacggtttgggtggtcacaacatcttcttggctgaagacttcaagggttccttcgacaccattttcgaagaattgggtttgccagctgacccatctttctatgttaacgttccatccagaattgacccttctgctgctccagaaggtaaggatgccattgtcatcttagtcccatgtggtcacatcgatgcttccaaccctcaagactacaacaaattggttgccagagccagaaagttcgtcatccaaaccttgtctgccaagttgggtctaccagatttcgaaaagatgattgttgctgaaaaggttcacgatgctccatcctgggaaaaggaattcaacttgaaggacggttccattttgggtttggctcacaacttcatgcaagtcttgggtttcagaccatccaccagacacccaaagtacgacaaattgttctttgtcggtgcttctacccacccaggtactggtgttccaattgtcttggctggtgccaaattgactgctaaccaagttttggaatccttcgatcgttctccagctccagatcctaacatgtctttgtctgttccatacggtaagccattgaaatccaacggtactggtattgactctcaagtccaattgaaattcatggacttggaacgttgggtttacctattagtcttgttgattggtgctgttatcgccagatccgtcggtgtcttggccttttaaaggaagtatctcggaaatattaatttaggccatgtccttatgcacgtttcttttgatacttacgggtacatgtacacaagtatatctatatatataaattaatgaaaatcccctatttatatatatgactttaacgagacagaacagttttttattttttatcctatttgatgaatgatacagtttcttattcacgtgttatacccacaccaaatccaatagcaataccggccatcacaatcactgtttcggcagcccctaagatcagacaaaacatccggaaccaccttaaatcaacgtccctcacgctttccggcatcttccagaccacagtatatccatccgcctcctgttg

SEQ ID NO: 8 - DNA, 3431 bp, Artificial Sequence
  Nucleotide sequence of the donor DNA crtYB expression cassette (conB-KlYDR2p-crtYB-ScPDC1t-conC)
cggatcgatgtacacaaccgactgcacccaaacgaacacaaatcttagcagtgcttttctttttttgcggtcacccccatgtggcggggaggcagaggagtaggtagagcaacgaatcctactatttatccaaattagtctaggaactctttttctagattttttagatttgagggcaagcgctgttaacgactcagaaatgtaagcactacggagtagaacgagaaatccgccataggtggaaatcctagcaaaatcttgcttaccctagctagcctcaggtaagctagccttagcctgtcaaatttttttcaaaatttggtaagtttctactagcaaagcaaacacggttcaacaaaccgaaaactccactcattatacgtggaaaccgaaacaaaaaaacaaaaaccaaaatactcgccaatgagaaagttgctgcgtttctactttcgaggaagaggaactgagaggattgactacgaaaggggcaaaaacgagtcgtattctcccattattgtctgctaccacgcggtctagtagaataagcaaccagtcaacgctaagacaggtaatcaaaataccagtctgctggctacgggctagtttttacctcttttagaacccactgtaaaagtccgttgtaaagcccgttctcactgttggcgtttttttttttttggtttagtttcttatttttcatttttttctttcatgaccaaaaacaaacaaatctcgcgatttgtactgcggccactggggcgtggccaaaaaaatgacaaatttagaaaccttagtttctgatttttcctgttatgaggagatatgataaaaaatattactgctttattgttttttttttatctactgaaatagagaaacttacccaaggaggaggcaaaaaaaagagtatatatacagcaggtaccattcagattttaatatattcttttctcttcttctacactattattataataattttactatattcatttttagcttaaaacctcatagaatattattcttcagtcactcgcttaaatacttatcaaaaatgaccgctttggcttactaccaaatccacttgatctacactttgccaatcttaggtttgctaggtttgttgacttctccaattttgaccaaattcgacatctacaagatttctatcttagtctttattgctttctctgctaccactccatgggactcctggatcatcagaaacggtgcctggacctacccatctgctgaatctggtcaaggtgttttcggtacctttttggatgtcccatacgaagaatacgccttctttgttatccaaaccgtcatcaccggtttggtttacgttttggctaccagacatttgttgccatctttggctctaccaaagacccgttcttctgccttgtctctagctttgaaggctttaatcccattgccaatcatctatttgttcaccgctcatccatctccatccccagatcctttggttactgaccactacttctacatgagagctttgtctttgttgatcaccccaccaaccatgttgttggctgctttatctggtgaatacgctttcgactggaaatctggtagagctaagtccaccattgctgccatcatgatcccaactgtctacttgatctgggttgactacgttgccgttggtcaagactcctggtccatcaacgatgaaaagattgtcggttggagattaggtggtgtcttgccaattgaagaagctatgttcttcttattgaccaacttgatgatcgttttgggtttgtctgcctgtgaccacactcaagccttgtacttgttgcacggtagaactatctacggtaacaagaagatgccatcttctttcccattaatcactccaccagttttgtccttgttcttctcctccagaccatactcctcccaaccaaagagagatttggaattggctgtcaagttgttggaaaagaagtccagatctttcttcgttgcttctgccggtttcccatctgaagtcagagaaagattggttggtttgtacgctttctgtcgtgtcaccgatgacttgattgactctccagaagtttcctccaacccacacgctaccattgacatggtttccgatttcttgactttattattcggtcctccattgcacccatctcaaccagacaagattttgtcttctccattattaccaccttcccacccatccagaccaactggtatgtacccattaccaccacctccatctttgtctccagctgaattggtccaattcttgactgaacgtgtcccagttcaataccacttcgctttcagattgttggccaaattgcaaggtttgattccaagatacccattggatgaattattgagaggttacaccactgacttgatcttcccattgtccactgaagccgtccaagctagaaagaccccaattgaaactactgctgacttgttggactacggtttgtgtgttgccggttctgttgctgaattgttggtctacgtttcctgggcttccgctccatcccaagttccagctactattgaagaaagagaagctgttttggtcgcctctcgtgaaatgggtaccgctttgcaattggtcaacattgccagagatatcaagggtgacgctactgaaggtagattctacttgccattgtctttctttggtttgagagatgaatccaaattggccattccaactgactggactgaaccaagacctcaagatttcgacaaattgttgtctctatctccatcttccactttaccatcctctaacgcttctgaatccttcagattcgaatggaagacctactctttgccattggttgcttacgctgaagatttggctaagcactcttacaagggtattgacagattaccaactgaagtccaagctggtatgagagctgcttgtgcttcttacttgttgattggtcgtgaaatcaaggttgtctggaagggtgatgtcggtgaaagaagaaccgttgctggttggagaagagtcagaaaggttttgtctgttgtcatgtccggttgggaaggtcaataaagcgatttaatctctaattattagttaaagttttataagcatttttatgtaacgaaaaataaattggttcatattattactgcactgtcacttaccatggaaagaccagacaagaagttgccgacagtctgttgaattggcctggttaggcttaagtctgggtccgcttctttacaaatttggagaatttctcttaaacgatatgtatattcttttcgttggaaaagatgtcttccaaaaaaaaaaccgatgaattagtggaaccaaggaaaaaaaaagaggtatccttgattaaggaacacctcacgctttccggcatcttccagaccacagtatatccatccgcctcctgttg

SEQ ID NO: 9 - DNA, 2758 bp, Artificial Sequence
  Nucleotide sequence of the donor DNA crtI expression cassette (conD-ScPRE3p-crtI-ScTAL1t-conE)
aacgttgtccaggtttgtatccacgtgtgtccgttccgccaatattccgcgtgccaaacattaatttgttctgcatactttgaacctttcagaaaataaaaaacattacgcgcatacttaccctgctcgcgaagaagagtaacactaacgcattctatgggcaattgaagacagtattcagtacaagacatagtccgtttccttgagtcaattcctatagcattatgaactagccgcctttaagagtgccaagctgttcaacaccgatcatttttgatgatttggcgtttttgttatattgatagatttcttttgaattttgtcattttcacttttccactcgcaacggaatccggtggcaaaaaagggaaaagcattgaaatgcaatctttaacagtattttaaacaagttgcgacacggtgtacaattacgataagaattgctacttcaaagtacacacagaaagttaacatgaatggaattcaagtggacatcaatcgtttgaaaaagggcgaagtcagtttaggtacctcaatgtatgtatataagaatttttcctcccactttattgtttctaaaagttcaatgaagtaaagtctcaattggccttattactaactaataggtatcttataatcacctaataaaatagaatgggtaaggaacaagaccaagacaagccaactgccatcatcgttggttgtggtatcggtggtattgctaccgctgccagattagctaaggaaggtttccaagttaccgtctttgaaaagaacgactactccggtggtagatgttctttgattgaaagagatggttacagattcgaccaaggtccatctttgttgctattaccagacttgttcaagcaaaccttcgaagatttgggtgaaaagatggaagactgggttgatttgatcaagtgtgaaccaaactacgtttgtcacttccatgatgaagaaactttcaccttctccactgacatggctttattgaagagagaagtcgaaagatttgaaggtaaagatggtttcgacagattcttgtctttcatccaagaagctcacagacattacgaattggctgttgtccacgtcttgcaaaagaacttcccaggtttcgctgctttcttgagattacaattcatcggtcaaatcttagctttgcacccatttgaatccatctggaccagagtttgtcgttacttcaagactgacagattgagaagagtcttctcctttgccgttatgtacatgggtcaatctccatactctgctccaggtacctactccttgttgcaatacactgaattgactgaaggtatctggtacccaagaggtggtttctggcaagttccaaacactttgttgcaaatcgtcaagagaaacaacccatctgctaagttcaacttcaacgctccagtttctcaagttttgttgtctccagctaaggacagagctaccggtgtcagattagaatctggtgaagaacaccacgctgatgttgtcattgtcaatgctgacttggtctacgcttctgaacatttgattccagatgatgctagaaacaagatcggtcaattaggtgaagttaagcgttcctggtgggctgatttggttggtggtaagaagttgaagggttcttgttcttctttgtctttctactggtctatggacagaatcgttgacggtttgggtggtcacaacatcttcttggctgaagacttcaagggttccttcgacaccattttcgaagaattgggtttgccagctgacccatctttctatgttaacgttccatccagaattgacccttctgctgctccagaaggtaaggatgccattgtcatcttagtcccatgtggtcacatcgatgcttccaaccctcaagactacaacaaattggttgccagagccagaaagttcgtcatccaaaccttgtctgccaagttgggtctaccagatttcgaaaagatgattgttgctgaaaaggttcacgatgctccatcctgggaaaaggaattcaacttgaaggacggttccattttgggtttggctcacaacttcatgcaagtcttgggtttcagaccatccaccagacacccaaagtacgacaaattgttctttgtcggtgcttctacccacccaggtactggtgttccaattgtcttggctggtgccaaattgactgctaaccaagttttggaatccttcgatcgttctccagctccagatcctaacatgtctttgtctgttccatacggtaagccattgaaatccaacggtactggtattgactctcaagtccaattgaaattcatggacttggaacgttgggtttacctattagtcttgttgattggtgctgttatcgccagatccgtcggtgtcttggccttttaaaggaagtatctcggaaatattaatttaggccatgtccttatgcacgtttcttttgatacttacgggtacatgtacacaagtatatctatatatataaattaatgaaaatcccctatttatatatatgactttaacgagacagaacagttttttattttttatcctatttgatgaatgatacagtttcttattcacgtgttatacccacaccaaatccaatagcaataccggccatcacaatcactgtttcggcagcccctaagatcagacaaaacatccggaaccaccttaaatcaacgtccctcaaataaccacaaacatccttcccatatgctcggtcgtgcttgttgtacct

SEQ ID NO: 10 - DNA, 22 bp, Artificial Sequence
  Nucleotide sequence of the FW primer to obtain the con5-crtE-conA donor DNA expression cassette, integration into INT1
aagcgacttccaatcgctttgc

SEQ ID NO: 11 - DNA, 21 bp, Artificial Sequence
  Nucleotide sequence of the REV primer to obtain the con5-crtE-conA donor DNA expression cassette, integration into INT1
aaagcaaaggaaggagagaac

SEQ ID NO: 12 - DNA, 76 bp, Artificial Sequence
  Nucleotide sequence of the FW primer to obtain the conB-crtYB-conC donor DNA expression cassette, integration into INT2
cggatcgatgtacacaaccgactgcacccaaacgaacacaaatcttagcagtgcttttctttttttgcggtcaccc

SEQ ID NO: 13 - DNA, 70 bp, Artificial Sequence
  Nucleotide sequence of the REV primer to obtain the conB-crtYB-conC donor DNA expression cassette, integration into INT2
caacaggaggcggatggatatactgtggtctggaagatgccggaaagcgttgttccttaatcaaggatac

SEQ ID NO: 14 - DNA, 71 bp, Artificial Sequence
  Nucleotide sequence of the FW primer to obtain the conD-crtI-conE donor DNA expression cassette, integration into INT3
aacgttgtccaggtttgtatccacgtgtgtccgttccgccaatattccgcacattaatttgttctgcatac

SEQ ID NO: 15 - DNA, 71 bp, Artificial Sequence
  Nucleotide sequence of the REV primer to obtain the conD-crtI-conE donor DNA expression cassette, integration into INT3
aggtacaacaagcacgaccgagcatatgggaaggatgtttgtggttatttgacgttgatttaaggtggttc

SEQ ID NO: 16 - DNA, 577 bp, Artificial Sequence
  Nucleotide sequence of the INT1 5′-con5 donor DNA flank sequence
cactatagcaatctggctatatgatatgcagaaaatagtgattactgtgctctttgctgcatcatgcatcatatgtttggcattctgtcctgtcagatgaatgggcgaaacaattcggggagctttgttgcgacttggtacccggcaagccgcgacctatattttgcattaaacgagttcatcgtggatttatcgccatgagccttagcttatcaagcctctcacagacaattaagcaatgaaaaaggtataccatcggcgcagaatggttaactagtgggttcatactgctgtgttatagattgttacctaagtgatcaccaaaaaaaagtgcaaaaaggaaaaaaaaataagagacaggtaacttccacaagcttattcttccaaaaatcaatcttatcttcatgccagcaatagttgcgtgctgagctcaacagtgcccaacccttgtgcaccgtagaattgtagaatacaaatacataaataagtgtgttcccgaaggactaaggaatgacggcagaggagtcaagcgacttccaatcgctttgcatatccagtaccacacccacaggcgttt

SEQ ID NO: 17 - DNA, 581 bp, Artificial Sequence
  Nucleotide sequence of the conA - INT1 3′ donor DNA flank sequence
ttgcccatcgaacgtacaagtactcctctgttctctccttcctttgctttaagcgttgaagtttcctctttgtatatttgagatcttcattttatcggattctttgtcatcagacaacttgttgagtggtactaaaggagtgcttttcatcatccttttggtgaacgatttcaaatacgttagtgttttctgagctagttttgatcaattcaggtgattcgttatcagaactctcaggtttgtattcgtgtccagttgtgtagcattcgcctaacgtgtaagcacggatttcttcctcagaaatttcactgtatggaatcatgcccttctttctcgcttcttcgtcggtaaatgcaccatagtaatctttgtcatcatgtctaacagtaattttgaatgggaagaagacacatagcccccagtaaacgaaaaaagaaatcaaaaaggagaagaaagaatcaccataaaagaatttaacaatacctgagtcgtggaaatagttattgttgacttcccaagcgataccaggtagaccgggagccataccacacacccaggcaacgatagctctccagttgaca

SEQ ID NO: 18 - DNA, 634 bp, Artificial Sequence
  Nucleotide sequence of the INT2 5′-conB donor DNA flank sequence
agaagatttctcttcaatctccttctccttaacacttaattttggcacttcgtcaactttaattttcttggaagccattgctatgtattcttagtgtactgcgacttgggtttctttcttgctttgtataaaagtgcgatgagttcaatcatcaagtgaatttttttttttcagcctgcattgaaaaaaaaaaaattgtatgctcgagcgatgggaaggttttttcctcttcccgttcaaagcttcctcggaccaactccactttttctcggcccgttgtaaagaaatgggtaagagaactaaattgcaataagcttcgaattatcgttttgatccagacataaagagatgtacaaactacaaaacaagagtaaaataactcgaacgaagggtacaccttctgtaataaaacaaattcactataactgccttattggagtgaaaaaacaagattatgtagaccccctctcgcgataaagaacattaatgactgcaacacccgcacaccacaaggtgaagggcaaaggtgaattaaccaaagtgaagaggacgacgtagaatcaaccaggtatccttgataagggcggatcgatgtacacaaccgactgcacccaaacgaacacaaatcttagca

SEQ ID NO: 19 - DNA, 551 bp, Artificial Sequence
  Nucleotide sequence of the conC-INT2 3′ donor DNA flank sequence
acgctttccggcatcttccagaccacagtatatccatccgcctcctgttgggcgattacacaagcggtggcgtcttgaaaaaagttgggtttcttatctgtacggcgatcactcccctcatatgatagctgtgtagagtgacttgctctttgattggccttacttcttgcatctatcatcctttcttttactctctccaatcaccacaactcatatcattctacatttcattttattgttctgtacaatttttaggtttgtaaaatatataagtgatgtacatatcttctatatcctttccgtttgtgtctattttagggttggcttattacgcattattcctactttatttttacttgcttacttgaaaaagaaggtataccacagtgctgctccactgcaaacaatataaaatattatcaattatttttttttttcttcttttgaattagatcactaataaactcttataatgctcaaccatcccagccaaggctcagatgatgctcaggacgaaaagcaaggcgacttcccggtcatcgaagaggaga

SEQ ID NO: 20 - DNA, 552 bp, Artificial Sequence
  Nucleotide sequence of the INT3 5′-conD donor DNA flank sequence
ggtcgtttttgtgcagcatattgtcctctagatgcaaactctgcaggtccatttgcagtaaagtgagttgcctctcgaagaatcattaatttcgtataaccgtcactattaaagtcagaaaataaattctgtcgtagacaatgttaccataatgttcttgtccattttgcatacactttaaatattcatttgatttctcagggttcatgatcataataaattgcgcattcgcaaggcggtagtattataatggggtccatcattctgtagcaagaagttacagtacgctgttcaagcgttaaacaagataagtaatctcgaatgaaacattcatatttcgcatgagccaacatacagttgctgagtaatcttcattgcgcttatttatcggcattgagattgtaaaggaagtaaaacgcatttttgcagatctgttctcttatgtatttttaatcgtccttgtatggaagtatcaaaggggacgttcttcacctccttggaaaacgttgtccaggtttgtatccacgtgtgtccgttccgccaatattccgc

SEQ ID NO: 21 - DNA, 550 bp, Artificial Sequence
  Nucleotide sequence of the conE-INT3 3′ donor DNA flank sequence
aaataaccacaaacatccttcccatatgctcggtcgtgcttgttgtacctgatgggacgtcagcactgtacttgtttttgcgactagattgtaaatcattctttatttaatctctttctttaactactgcttaaagtataatttggtccgtagtttaataactatactaagcgtaacaatgcatactgacattataagcctgaacattacgagtttaagttgtatgtaggcgttctgtaagaggttactgcgtaaattatcaacgaatgcattggtgtatttgcgaaagctacttcttttaacaagtatttacataagaataatggtgatctgctcaactgatttggtgataactctaacttttttagcaacaatttaaaagataattcgaacatatataacagtaggaagaatttgtgtacgtcaaattaagataatttagcattaccaaagttattaacctaaacataaaatatatatgagacacatgtggaaatcgtatgaaacaactgttatgaaactgacaagaatgaatatatagagtaagctc

SEQ ID NO: 22 - DNA, 23 bp, Artificial Sequence
  Nucleotide sequence of the FW primer to obtain the INT1 5′-con5 donor flank sequence
cactatagcaatctggctatatg

SEQ ID NO: 23 - DNA, 70 bp, Artificial Sequence
  Nucleotide sequence of the REV primer to obtain the INT1 5′-con5 donor flank sequence
aaacgcctgtgggtgtggtactggatatgcaaagcgattggaagtcgcttgactcctctgccgtcattcc

SEQ ID NO: 24 - DNA, 72 bp, Artificial Sequence
  Nucleotide sequence of the FW primer to obtain the conA-INT1 3′ donor flank sequence
ttgcccatcgaacgtacaagtactcctctgttctctccttcctttgctttaagcgttgaagtttcctctttg

SEQ ID NO: 25 - DNA, 20 bp, Artificial Sequence
  Nucleotide sequence of the REV primer to obtain the conA-INT1 3′ donor flank sequence
tgtcaactggagagctatcg

SEQ ID NO: 26 - DNA, 21 bp, Artificial Sequence
  Nucleotide sequence of the FW primer to obtain the INT2 5′-conB donor flank sequence
agaagatttctcttcaatctc

SEQ ID NO: 27 - DNA, 73 bp, Artificial Sequence
  Nucleotide sequence of the REV primer to obtain the INT2 5′-conB donor flank sequence
tgctaagatttgtgttcgtttgggtgcagtcggttgtgtacatcgatccgcccttatcaaggatacctggttg

SEQ ID NO: 28 - DNA, 70 bp, Artificial Sequence
  Nucleotide sequence of the FW primer to obtain the conC-INT2 3′ donor flank sequence
acgctttccggcatcttccagaccacagtatatccatccgcctcctgttgggcgattacacaagcggtgg

SEQ ID NO: 29 - DNA, 20 bp, Artificial Sequence
  Nucleotide sequence of the REV primer to obtain the conC-INT2 3′ donor flank sequence
tctcctcttcgatgaccggg

SEQ ID NO: 30 - DNA, 23 bp, Artificial Sequence
  Nucleotide sequence of the FW primer to obtain the INT3 5′-conD donor flank sequence
ggtcgtttttgtgcagcatattg

SEQ ID NO: 31 - DNA, 70 bp, Artificial Sequence
  Nucleotide sequence of the REV primer to obtain the INT3 5′-conD donor flank sequence
gcggaatattggcggaacggacacacgtggatacaaacctggacaacgttttccaaggaggtgaagaacg

SEQ ID NO: 32 - DNA, 71 bp, Artificial Sequence
  Nucleotide sequence of the FW primer to obtain the conE-INT3 3′ donor flank sequence
aaataaccacaaacatccttcccatatgctcggtcgtgcttgttgtacctgatgggacgtcagcactgtac

SEQ ID NO: 33 - DNA, 23 bp, Artificial Sequence
  Nucleotide sequence of the REV primer to obtain the conE-INT3 3′ donor flank sequence
gagcttactctatatattcattc

SEQ ID NO: 34 - DNA, 22 bp, Artificial Sequence
  Nucleotide sequence of the FW primer to check correct integration of crtE at the INT1 locus 5′ end (band A)
gttactaaactggaactgtccg

SEQ ID NO: 35 - DNA, 20 bp, Artificial Sequence
  Nucleotide sequence of the REV primer to check correct integration of crtE at the INT1 locus 5′ end (band A)
ccttgtccaagtaggtgtcc

SEQ ID NO: 36 - DNA, 22 bp, Artificial Sequence
  Nucleotide sequence of the FW primer to check correct integration of crtE at the INT1 locus 3′ end (band B)
cactgctaactacgtttacttc

SEQ ID NO: 37 - DNA, 21 bp, Artificial Sequence
  Nucleotide sequence of the REV primer to check correct integration of crtE at the INT1 locus 3′ end (band B)
gctgtcatgatctgtgataac

SEQ ID NO: 38 - DNA, 21 bp, Artificial Sequence
  Nucleotide sequence of the FW primer to check correct integration of crtI at the INT3 locus 5′ end (band C)
ctctcatgaagcagtcaagtc

SEQ ID NO: 39 - DNA, 21 bp, Artificial Sequence
  Nucleotide sequence of the REV primer to check correct integration of crtI at the INT3 locus 5′ end (band C)
ccttaccttctggagcagcag

SEQ ID NO: 40 - DNA, 20 bp, Artificial Sequence
  Nucleotide sequence of the FW primer to check correct integration of crtI at the INT3 locus 3′ end (band D)
gatcggtcaattaggtgaag

SEQ ID NO: 41 - DNA, 21 bp, Artificial Sequence
  Nucleotide sequence of the REV primer to check correct integration of crtI at the INT3 locus 3′ end (band D)
ctggttacttccctaagactg

SEQ ID NO: 42 - DNA, 20 bp, Artificial Sequence
  Nucleotide sequence of the FW primer to check correct integration of crtYB at the INT2 locus 5′ end (band E)
cactggaacttgagcttgag

SEQ ID NO: 43 - DNA, 21 bp, Artificial Sequence
  Nucleotide sequence of the REV primer to check correct integration of crtYB at the INT2 locus 5′ end (band E)
ctggcaatgttgaccaattgc

SEQ ID NO: 44 - DNA, 20 bp, Artificial Sequence
  nucleotide sequence of the FW primer to check correct integration of crtYB at the NINT2 locus 3′ end (band F)
gtctccagctgaattggtcc

SEQ ID NO: 45 - DNA, 20 bp, Artificial Sequence
  Nucleotide sequence of the REV primer to check correct integration of crtYB at the INT2 locus 3′ end (band F)
ccaacgtgccttaaagtctg

SEQ ID NO: 46 - DNA, 21 bp, Artificial Sequence
  Nucleotide sequence of the FW primer to remove SapI restriction site in pRN1120
tattgggccctcttccgcttc

SEQ ID NO: 47 - DNA, 18 bp, Artificial Sequence
  Nucleotide sequence of the REV primer to remove SapI restriction site in pRN1120
cgcaaaccgcctctcccc

SEQ ID NO: 48 - DNA, 547 bp, Artificial Sequence
  Nucleotide sequence of the gBlock to enable direct SapI cloning of a genomic target for SpCas9
ggagctagcatgcggccgctctagaactagtggatcccccgggctgcaggaattctctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcagaagagcctgaggtcgacggtatcgataagcttgatatcaattccccgggggatccactgctcttctgttttagagctagaaatagcaagttaaaataaggctagtccgttatcaacttgaaaaagtggcaccgagtcggtggtgctttttttgttttttatgtctctcgagggggggcccggtacccagcttttgttccctttagtgagggttaattccga

SEQ ID NO: 49 - DNA, 6121 bp, Artificial Sequence
  Nucleotide sequence of the nucleotide sequence of vector pGRN002
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacacctaggatccgtcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactaggggcagggcatgctcatgtagagcgcctgctcgccgtccgaggcggtgccgtcgtacagggcggtgtccaggccgcagagggtgaaccccatccgccggtacgcgtggatcgccggtgcgttgacgttggtgacctccagccagaggtgcccggcgccccgctcgcgggcgaactccgtcgcgagccccatcaacgcgcgcccgaccccgtgcccccggtgctccggggcgacctcgatgtcctcgacggtcagccggcggttccagccggagtacgagacgaccacgaagcccgccaggtcgccgtcgtccccgtacgcgacgaacgtccgggagtccgggtcgccgtcctccccggcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcattttacatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggccttaagttcgaacgtacgagctccggcattgcgaataccgctttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactaggtatagtctatagtccgtggaattaattctcatgtttgacagcttatcatcgataatccggagctagcatgcggccgctctagaactagtggatcccccgggctgcaggaattctctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcagaagagcctgaggtcgacggtatcgataagcttgatatcaattccccgggggatccactgctcttctgttttagagctagaaatagcaagttaaaataaggctagtccgttatcaacttgaaaaagtggcaccgagtcggtggtgctttttttgttttttatgtctctcgagggggggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 50 - DNA, 52 bp, Artificial Sequence
  Nucleotide sequence of the FW oligonucleotide named FW oligo 1 for oligo assembly variant 1 and 2
gcagtgaaagataaatgatcaatttctactaagtgtagatctggtgggagag

SEQ ID NO: 51 - DNA, 44 bp, Artificial Sequence
  Nucleotide sequence of the FW oligonucleotide named FW oligo 2 for oligo assembly variant 1 and 2
aaagcttatgaaatttctactaagtgtagatgtgccgtacgccg

SEQ ID NO: 52 - DNA, 43 bp, Artificial Sequence
  Nucleotide sequence of the FW oligonucleotide named FW oligo 3 for oligo assembly variant 1 and 2
gagccgacggaatttctactaagtgtagattgcccctcttata

SEQ ID NO: 53 - DNA, 50 bp, Artificial Sequence
  Nucleotide sequence of the FW oligonucleotide named FW oligo 4 for oligo assembly variant 1 and 2
cgattatatttttttttgttttttatgtctggggggcccggtacccagct

SEQ ID NO: 54 - DNA, 30 bp, Artificial Sequence
  Nucleotide sequence of the REV oligonucleotide named REV oligo 1 for oligo assembly variant 1
agtagaaattgatcatttatctttcactgc

SEQ ID NO: 55 - DNA, 43 bp, Artificial Sequence
  Nucleotide sequence of the REV oligonucleotide named REV oligo 2 for oligo assembly variant 1
agtagaaatttcataagctttctctcccaccagatctacactt

SEQ ID NO: 56 - DNA, 43 bp, Artificial Sequence
  Nucleotide sequence of the REV oligonucleotide named REV oligo 3 for oligo assembly variant 1 and 2
agtagaaattccgtcggctccggcgtacggcacatctacactt

SEQ ID NO: 57 - DNA, 73 bp, Artificial Sequence
  Nucleotide sequence of the REV oligonucleotide named REV oligo 4 for oligo assembly variant 1 and 2
agctgggtaccgggccccccagacataaaaaacaaaaaaaaatataatcgtataagaggggcaatctacactt

SEQ ID NO: 58 - DNA, 73 bp, Artificial Sequence
  Nucleotide sequence of the REV oligonucleotide named REV oligo 5 for oligo assembly variant 2
agtagaaatttcataagctttctctcccaccagatctacacttagtagaaattgatcatttatctttcactgc

SEQ ID NO: 59 - DNA, 78 bp, Artificial Sequence
  Nucleotide sequence of the 5′ homology to vector pRN1120 part of the LbCpf1_crRNA_array shown in SEQ ID NO 2
catgtttgacagcttatcatcgataatccggagctagcatgcggccgctctagaactagtggatcccccgggctgcag

SEQ ID NO: 60 - DNA, 269 bp, Artificial Sequence
  Nucleotide sequence of the SNR52p RNA pol III promoter part of the LbCpf1_crRNA_array shown in SEQ ID NO 2
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatc

SEQ ID NO: 61 - DNA, 20 bp, Artificial Sequence
  Nucleotide sequence of the direct repeat (specific for LbCpf1) part of the LbCpf1_crRNA_array shown in SEQ ID NO 2
aatttctactaagtgtagat

SEQ ID NO: 62 - DNA, 23 bp, Artificial Sequence
  Nucleotide sequence of the genomic target / spacer of the INT1 integration site part of the LbCpf1_crRNA_array shown in SEQ ID NO 2
ctggtgggagagaaagcttatga

SEQ ID NO: 63 - DNA, 23 bp, Artificial Sequence
  Nucleotide sequence of the genomic target / spacer of the INT2 integration site part of the LbCpf1_crRNA_array shown in SEQ ID NO 2
gtgccgtacgccggagccgacgg

SEQ ID NO: 64 - DNA, 23 bp, Artificial Sequence
  Nucleotide sequence of the genomic target / spacer of the INT3 integration site part of the LbCpf1_crRNA_array shown in SEQ ID NO 2
tgcccctcttatacgattatatt

SEQ ID NO: 65 - DNA, 20 bp, Artificial Sequence
  Nucleotide sequence of the SUP4 3′ terminator part of the LbCpf1_crRNA_array shown in SEQ ID NO 2
tttttttgttttttatgtct

SEQ ID NO: 66 - DNA, 87 bp, Artificial Sequence
  Nucleotide sequence of the 3′ homology to vector pRN1120 part of the LbCpf1_crRNA_array shown in SEQ ID NO 2
ggggggcccggtacccagcttttgttccctttagtgagggttaattccgagcttggcgtaatcatggtcatagctgtttcctgtgtg

SEQ ID NO: 67 - DNA, 28 bp, Artificial Sequence
  Nucleotide sequence of the Csy4 recognition site for the Csy4 endoribonuclease from Pseudomonas aeruginosa
gttcactgccgtataggcagctaagaaa

SEQ ID NO: 68 - DNA, 87 bp, Artificial Sequence; identical to SEQ ID NO: 66
  coding sequence of tRNAgly

SEQ ID NO: 69 - DNA, 10 bp, Artificial Sequence
  5′ leader sequence for the coding sequence of tRNAgly
aaaataataa

SEQ ID NO: 70 - DNA, 72 bp, Artificial Sequence
  coding sequence of tRNAglu
tccgatatagtgtaacggctatcacatcacgctttcaccgtggagaccggggttcgactccccgtatcggag

SEQ ID NO: 71 - DNA, 10 bp, Artificial Sequence
  5′ leader sequence for the coding sequence of tRNAglu
ttaattatca

SEQ ID NO: 72 - DNA, 73 bp, Artificial Sequence
  coding sequence of tRNAtyr (tAGC)
gggcgtgtggcgtagtcggtagcgcgctcccttagcatgggagaggtctccggttcgattccggactcgtcca

SEQ ID NO: 73 - DNA, 10 bp, Artificial Sequence
  5? leader sequence for the coding sequence of tRNAtyr
acagaaaatc

SEQ ID NO: 74 - DNA, 72 bp, Artificial Sequence
  coding sequence of tRNAarg (tCTT)
gttccgttggcgtaatggtaacgcgtctccctcctaaggagaagactgcgggttcgagtcccgtacggaacg

SEQ ID NO: 75 - DNA, 10 bp, Artificial Sequence
  5? leader sequence of the coding sequence of tRNAarg
caacgaaata

SEQ ID NO: 76 - DNA, 74 bp, Artificial Sequence
  coding sequence of tRNAasn (tGTT)
gactccatggccaagttggttaaggcgtgcgactgttaatcgcaagatcgtgagttcaaccctcactggggtcg

SEQ ID NO: 77 - DNA, 10 bp, Artificial Sequence
  5? leader sequence for the coding sequence of tRNAasn
aagataaagt

SEQ ID NO: 78 - DNA, 74 bp, Artificial Sequence
  coding seqeunce of tRNAile (tAAT)
ggtctcttggcccagttggttaaggcaccgtgctaataacgcggggatcagcggttcgatcccgctagagacca

SEQ ID NO: 79 - DNA, 10 bp, Artificial Sequence
  5′ leader sequence for the coding sequence of tRNAile
acaaaagaat